(19) United States
(12) Patent Application Publication
(10) Pub. No.: US 2010/0009934 A1
(43) Pub. Date: Jan. 14, 2010

(54) BETA ADRENERGIC RECEPTOR AGONISTS FOR THE TREATMENT OF B-CELL PROLIFERATIVE DISORDERS

(75) Inventors: Richard RICKLES, Arlington, MA (US); Margaret S. Lee, Middleton, MA (US)

Correspondence Address: CLARK & ELBING LLP, 101 FEDERAL STREET, BOSTON, MA, 02110, US

(73) Assignee: CombinatoRx, Incorporated, Cambridge, MA (US)

(21) Appl. No.: 12/480,034

(22) Filed: Jun. 8, 2009

Related U.S. Application Data
(60) Provisional application No. 61/060,064, filed on Jun. 9, 2008.

(51) Int. Cl. A61K 31/69; A61K 31/505; A61K 31/573; A61K 31/135; A61K 31/137; A61K 31/195; A61K 31/351; A61K 31/454

(52) U.S. Cl. 514/64; 514/274; 514/179; 514/651; 514/653; 514/564; 514/459; 514/323

(57) ABSTRACT

The invention features a method of treating a B-cell proliferative disorder by administering to a patient a BAR agonist, e.g., formulated for administration by a route other than inhalation (such as for oral or intravenous administration), in an amount effective to treat the B-cell proliferative disorder. The BAR agonist may be administered as a monotherapy or in combination with one or more other agents, e.g., a PDE inhibitor, an A2A receptor agonist, or an antiproliferative compound, in amounts that together are effective to treat the B-cell proliferative disorder. The invention further features pharmaceutical compositions and kits including a BAR agonist, alone or in combination with additional agents, for the treatment of a B-cell proliferative disorder.

### CROSS-REFERENCE TO RELATED APPLICATIONS

This application claims priority to U.S. Provisional Application No. 61/060,064, filed Jun. 9, 2008, which is hereby incorporated by reference.

### BACKGROUND OF THE INVENTION

The invention relates to the field of treatments for proliferative disorders.

Multiple Myeloma (MM) is a malignant disorder of antibody producing B-cells. MM cells flourish in the bone marrow microenvironment, generating tumors called plasmacytomas that disrupt haematopoesis and cause severe destruction of bone. Disease complications include anemia, infections, hypercalcemia, organ dysfunction, and bone pain.

For many years, the combination of glucocorticoids (e.g., dexamethasone or prednisolone) and alkylating agents (e.g., melphalan) was standard treatment for MM, with glucocorticoids providing most of the clinical benefit. In recent years, treatment options have advanced with three drugs approved by the FDA—Velcade™ (bortezomib), thalidomide, and lenalidomide. Glucocorticoids remain the mainstay of treatment and are usually deployed in combination with FDA-approved or emerging drugs. Unfortunately, despite advances in the treatment, MM remains an incurable disease with most patients eventually succumbing to the cancer.

### SUMMARY OF THE INVENTION

In general, the invention features methods and composition employing a beta adrenergic receptor agonist (“BAR agonist”) for the treatment of a B-cell proliferative disorder.

Accordingly, in one aspect, the invention features a method of treating a B-cell proliferative disorder by administering to a patient a BAR agonist, e.g., formulated for administration by a route other than inhalation (such as for oral or intravenous administration), in an amount effective to treat the B-cell proliferative disorder.

The BAR agonist may be administered as a monotherapy or in combination with one or more other agents, e.g., a PDE inhibitor, an A2A receptor agonist, or an antiproliferative compound, in amounts that together are effective to treat the B-cell proliferative disorder.

The BAR agonist may also be administered with IL-6 to the patient. If not by direct administration of IL-6, patients may be treated with agent(s) to increase the expression or activity of IL-6. Such agents may include other cytokines (e.g., IL-1 or TNF), soluble IL-6 receptor α (sIL-6R α), platelet-derived growth factor, prostaglandin E1, forskolin, cholera toxin, dibutyryl cAMP, or IL-6 receptor agonists, e.g., the agonist antibody MT-18, K-7/D-6, and compounds disclosed in U.S. Pat. Nos. 5,914,106, 5,506,107, and 5,891,998.

The individual components of a combination may be administered simultaneously or within a specified period of time, e.g., 28 days.

The invention further features a pharmaceutical composition including a BAR agonist in an amount effective to treat a B-cell proliferative disorder, e.g., wherein the BAR agonist is formulated for administration by a route other than inhalation (such as for oral or intravenous administration). The composition may further include an A2A agonist, PDE inhibitor, IL-6 agonist, or antiproliferative compound in an amount in combination with the BAR agonist that is effective to treat a B-cell proliferative disorder. The pharmaceutical composition may further include a pharmaceutically acceptable excipient.

The invention also features a kit comprising a BAR agonist and an A2A agonist, PDE inhibitor, IL-6 agonist, or antiproliferative compound in amounts that together are effective to treat a B-cell proliferative disorder. In the kits of the invention, the BAR agonist may be formulated for administration by a route other than inhalation, e.g., oral or intravenous administration. Kits of the invention may further include instructions for administering the BAR agonist or combination of agents for treatment of the B-cell proliferative disorder.

Exemplary BAR agonists are beta 2 agonists. Examples of BAR agonists include arbutaline, arfomoterol, bambuterol, bitolterol, broxaterol, clenbuterol, fenoterol, formoterol, hexoprenaline, indacaterol, isoetharine, isoproterenol, levalbuterol, meluadrine, metaproterenol, nylidrin, picumeterol, pirbuterol, procaterol, reproterol, rimiterol, ritodrine, salbutamol, salmeterol, tulobuterol, terbutaline, and xamoterol. Additional BAR agonists are provided in Tables 1 and 2 herein. Exemplary A2A agonists, PDE inhibitors, and antiproliferative compounds are provided herein, e.g., in Tables 3-8. Exemplary combinations of BAR agonists and antiproliferative compounds are provided in Table 9.

In certain embodiments, when the B-cell proliferative disorder is B-CLL, the BAR agonist is not formoterol, isoproterenol, or salmeterol, and when the B-cell proliferative disorder is doxorubicin resistant multiple myeloma, the BAR agonist is not salbutamol. In other embodiments, the BAR agonist is not isoproterenol. In further embodiments, when the B-cell proliferative disorder is mantle cell lymphoma, the BAR agonist is not salmeterol administered with CHOP or bortezomib; when the B-cell proliferative disorder is multiple myeloma, the BAR agonist is not salbutamol administered with VAD; when the B-cell proliferative disorder is multiple myeloma, the BAR agonist is not salmeterol administered with prednisone and melphalan; when the B-cell proliferative disorder is multiple myeloma, the BAR agonist is not salbutamol administered with clodronate; or when the B-cell proliferative disorder is multiple myeloma, the BAR agonist is not salbutamol administered with melphalan, prednisone, and pamidronate for multiple myeloma.

In other embodiments, the patient is not suffering from asthma, bronchiolitis obliterans, COPD, shortness of breath, or an immunoinflammatory disorder (e.g., of the lungs). The patient may also be one not preparing to undergo, not undergoing, or not recovering from allogenic or autologous stem cell replacement. In other embodiments, the patient is not concomitantly treated with a stem cell mobilizer or an mTOR inhibitor and capecitabine. Compositions and kits of the invention may explicitly exclude a stem cell mobilizer or an mTOR inhibitor and capecitabine.

By “beta adrenergic receptor agonist” or “BAR agonist” is meant any member of the class of compounds that agonize a beta adrenergic receptor, as can be determined by assays well known in the art, see, e.g., Beta2-Agonists in Asthma Treatment, Pauwels and O'Byrne, Eds., Marcel Dekker 1997. Exemplary BAR agonists for use in the invention are described herein. A BAR agonist may be a beta 1 agonist, a beta 2 agonist, or a beta 3 agonist. In certain embodiments, a BAR agonist of the invention is specific to the beta 2 adrenergic receptor, e.g., has activity at the beta 2 adrenergic receptor that is at least 2, 5, 10, 20, 50, or 100 times greater than at the beta 1 and/or beta 3 adrenergic receptor. In other embodiments, the BAR agonist has activity at a beta adrenergic receptor that is at least 2, 5, 10, 20, 50, 100, 500, or 1000 times greater than at any alpha adrenergic receptor.

By “beta 2 agonist” is meant a BAR agonist whose antiproliferative effect on MM.1S cells is reduced in the presence of a selective beta 2 adrenergic receptor antagonist (for example, ICI 118,551 or butaxomine). In certain embodiments, the antiproliferative effect of a beta 2 agonist in MM.1S cells (used at a concentration equivalent to the Kᵢ) is reduced by at least 10, 20, 30, 40, 50, 60, 70, 80, or 90% by a selective beta 2 adrenergic receptor antagonist used at a concentration of at least 10-fold higher than its Kᵢ.

By “A2A receptor agonist” is meant any member of the class of compounds whose antiproliferative effect on MM.1S cells is reduced in the presence of an A2A-selective antagonist, e.g., SCH 58261. In certain embodiments, the antiproliferative effect of an A2A receptor agonist in MM.1S cells (used at a concentration equivalent to the Kᵢ) is reduced by at least 10, 20, 30, 40, 50, 60, 70, 80, or 90% by an A2A antagonist used at a concentration of at least 10-fold higher than its Kᵢ (for example, SCH 58261 (Kᵢ=5 nM) used at 78 nM)). An A2A receptor agonist may also retain at least 10, 20, 30, 40, 50, 60, 70, 80, 90, or 95% of its antiproliferative activity in MM.1S cells in the presence of an A1 receptor antagonist (e.g., DPCPX (89 nM)), an A2B receptor antagonist (e.g., MRS 1574 (89 nM)), an A3 receptor antagonist (e.g., MRS 1523 (87 nM)), or a combination thereof. In certain embodiments, the reduction of agonist-induced antiproliferative effect by an A2A antagonist will exceed that of an A1, A2B, or A3 antagonist. Exemplary A2A Receptor Agonists for use in the invention are described herein.

By “PDE inhibitor” is meant any member of the class of compounds having an IC₅₀ of 100 μM or lower concentration for a phosphodiesterase. In preferred embodiments, the IC₅₀ of a PDE inhibitor is 40, 20, 10 μM or lower concentration. In particular embodiments, a PDE inhibitor of the invention will have activity against PDE 2, 3, 4, or 7 or combinations thereof in cells of the B-type lineage. In preferred embodiments, a PDE inhibitor has activity against a particular type of PDE when it has an IC₅₀ of 40 μM, 20 μM, 10 μM, 5 μM, 1 μM, 100 nM, 10 nM, or lower concentration. When a PDE inhibitor is described herein as having activity against a particular type of PDE, the inhibitor may also have activity against other types, unless otherwise stated. Exemplary PDE inhibitors for use in the invention are described herein.

By “B-cell proliferative disorder” is meant any disease where there is a disruption of B-cell homeostasis leading to a pathologic increase in the number of B cells. A B-cell cancer is an example of a B-cell proliferative disorder. A B-cell cancer is a malignancy of cells derived from lymphoid stem cells and may represent any stage along the B-cell differentiation pathway. B-cell proliferative disorders include autoimmune lymphoproliferative disease, B-cell chronic lymphocytic leukemia (CLL), B-cell prolymphocyte leukemia, lymphoplasmacytic lymphoma, mantle cell lymphoma, follicular lymphoma, extranodal marginal zone B-cell lymphoma of mucosa-associated lymphoid tissue (MALT type), nodal marginal zone lymphoma, splenic marginal zone lymphoma, hairy cell leukemia, plasmacytoma, diffuse large B-cell lymphoma, Burkitt lymphoma, multiple myeloma, indolent myeloma, smoldering myeloma, monoclonal gammopathy of unknown significance (MGUS), B-cell non-Hodgkin's lymphoma, small lymphocytic lymphoma, monoclonal immunoglobin deposition diseases, heavy chain diseases, mediastinal (thymic) large B-cell lymphoma, intravascular large B-cell lymphoma, primary effusion lymphoma, lymphomatoid granulomatosis, precursor B-lymphoblastic leukemia/lymphoma, Hodgkin's lymphoma (e.g., nodular lymphocyte predominant Hodgkin's lymphoma, classical Hodgkin's lymphoma, nodular sclerosis Hodgkin's lymphoma, mixed cellularity Hodgkin's lymphoma, lymphocyte-rich classical Hodgkin's lymphoma, and lymphocyte depleted Hodgkin's lymphoma), post-transplant lymphoproliferative disorder, and Waldenstrom's macroglobulinemia.

By “effective” is meant the amount or amounts of one or more compounds sufficient to treat a B-cell proliferative disorder in a clinically relevant manner. An effective amount of an active varies depending upon the manner of administration, the age, body weight, and general health of the patient. Ultimately, the prescribers will decide the appropriate amount and dosage regimen. Additionally, an effective amount can be that amount of compound in a combination of the invention that is safe and efficacious in the treatment of a patient having the B-cell proliferative disorder as determined and approved by a regulatory authority (such as the U.S. Food and Drug Administration).

By “treating” is meant administering or prescribing a pharmaceutical composition for the treatment or prevention of a B-cell proliferative disorder.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, disregulation of the immune system, and unwanted proliferation of cells. Examples of immunoinflammatory disorders are acne vulgaris; acute respiratory distress syndrome; Addison's disease; adrenocortical insufficiency; adrenogenital ayndrome; allergic conjunctivitis; allergic rhinitis; allergic intraocular inflammatory diseases, ANCA-associated small-vessel vasculitis; angioedema; ankylosing spondylitis; aphthous stomatitis; arthritis, asthma; atherosclerosis; atopic dermatitis; autoimmune disease; autoimmune hemolytic anemia; autoimmune hepatitis; Behcet's disease; Bell's palsy; berylliosis; bronchial asthma; bullous herpetiformis dermatitis; bullous pemphigoid; carditis; celiac disease; cerebral ischaemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; epicondylitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; hypersensitivity drug reactions; idiopathic cerato-scleritis; idiopathic pulmonary fibrosis; idiopathic thrombocytopenic purpura; inflammatory bowel or gastrointestinal disorders, inflammatory dermatoses; juvenile rheumatoid arthritis; laryngeal edema; lichen planus; Loeffler's syndrome; lupus nephritis; lupus vulgaris; lymphomatous tracheobronchitis; macular edema; multiple sclerosis; musculoskeletal and connective tissue disorder; myasthenia gravis; myositis; obstructive pulmonary disease; ocular inflammation; organ transplant rejection; osteoarthritis; pancreatitis; pemphigoid gestationis; pemphigus vulgaris; polyarteritis nodosa; polymyalgia rheumatica; primary adrenocortical insufficiency; primary billiary cirrhosis; pruritus scroti; pruritis/inflammation, psoriasis; psoriatic arthritis; Reiter's disease; relapsing polychondritis; rheumatic carditis; rheumatic fever; rheumatoid arthritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; serum sickness; shoulder tendinitis or bursitis; Sjogren's syndrome; Still's disease; stroke-induced brain cell death; Sweet's disease; systemic dermatomyositis; systemic lupus erythematosus; systemic sclerosis; Takayasu's arteritis; temporal arteritis; thyroiditis; toxic epidermal necrolysis; tuberculosis; type-1 diabetes; ulcerative colitis; uveitis; vasculitis; and Wegener's granulomatosis. “Non-dermal inflammatory disorders” include, for example, rheumatoid arthritis, inflammatory bowel disease, asthma, and chronic obstructive pulmonary disease. “Dermal inflammatory disorders” or “inflammatory dermatoses” include, for example, psoriasis, acute febrile neutrophilic dermatosis, eczema (e.g., asteatotic eczema, dyshidrotic eczema, vesicular palmoplantar eczema), balanitis circumscripta plasmacellularis, balanoposthitis, Behcet's disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis. By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis. As will be appreciated by one skilled in the art, a particular disease, disorder, or condition may be characterized as being both a proliferative skin disease and an inflammatory dermatosis. An example of such a disease is psoriasis.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition.

By a “high dosage” is meant at least 5% (e.g., at least 10%, 20%, 50%, 100%, 200%, or even 300%) more than the highest standard recommended dosage of a particular compound for treatment of any human disease or condition.

Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein.

Other features and advantages of the invention will be apparent from the following detailed description, and from the claims.

### BRIEF DESCRIPTION OF THE DRAWINGS

FIG. 1 is a graph showing the induction of apoptosis after human multiple myeloma cells (MM.1S) were exposed to salmeterol and dexamethasone as single agents and in combination (48 hour timepoint).

FIG. 2 is a graph showing the induction of apoptosis after human multiple myeloma cells (MM.1S) were exposed to salmeterol and lenalidomide or trequinsin as single agents and in combination (72 hour timepoint).

FIG. 3 is a graph showing the induction of apoptosis after human multiple myeloma cells (MM.1S) were exposed to salmeterol and bortezomib as single agents and in combination (72 hour timepoint).

FIG. 4 is a graph of the antiproliferative activity of beta 2 agonist salbutamol against human multiple myeloma cells (MM.1R) after transfection of siRNA.

FIG. 5 is a graph of an annexin V/PI assay of control MM.1S cells and cells cultured in the presence of salmeterol or salbutamol for one month after exposure to dexamethasone and salmeterol.

FIG. 6 is a graph of the viability of patient multiple myeloma tumor cells treated with dexamethasone (DEX) and salmeterol.

FIG. 7 is a graph of the viability of patient multiple myeloma tumor cells treated with bortezomib (Bort) and salmeterol.

FIG. 8 is a graph of tumor volume in control and drug treated animals (salmeterol, dexamethasone, and salmeterol/dexamethasone) using the MM.1S tumor model.

FIG. 9 is a graph of tumor volume in control and drug treated animals (salmeterol, bortezomib and salmeterol/bortezomib) using the RPMI-8226 tumor model.

FIG. 10 is a graph of regression of tumor growth analysis of the activities of salmeterol, dexamethasone, and bortezomib as single agents and in combination using the RPMI-8226 tumor model.

FIG. 11 is a graph of analysis of the effects of salmeterol, dexamethasone, and bortezomib on mouse body weight when deployed as single agents and in combination using the RPMI-8226 tumor model.

### DETAILED DESCRIPTION OF THE INVENTION

We have identified compounds that display synergistic anti-proliferative activity when used in combination with drugs deployed in the clinic for the treatment of multiple myeloma. BAR agonists are highly synergistic with multiple myeloma standard of care (dexamethasone, lenalidomide, melphalan, doxorubicin, and bortezomib). BAR agonists also synergize with adenosine A2A receptors agonists and PDE inhibitors. The synergistic activities observed with BAR agonists are observed when tested on various multiple myeloma cell lines, as well as OCI-ly10, a diffuse large B-cell lymphoma (DLBCL) cell line, and GA-10, a Burkitt's lymphoma cell line.

Accordingly, the invention features methods, compositions, and kits for the administration of an effective amount of a BAR agonist, alone or in combination with one or more additional agents, to treat a B-cell proliferative disorder. The invention is described in greater detail below.

### Beta Adrenergic Receptor Agonists

Exemplary BAR agonists for use in the invention are shown in Table 1.

TABLE 1

| BAR Agonist | Synonym |
|---|---|
| (−)-pindolol | 1-(Indol-4-yloxy)-3-(isopropylamino)-2-propanol |
| 3H-SB-206606 | tritiated (4-(2-((2-(3-chlorophenyl)-2-hydroxyethyl)amino)propyl)phenoxy) |
| | acetic acid |
| Amibegron | Ethyl {[(7S)-7-{[(2R)-2-(3-chlorophenyl)-2-hydroxyethyl]amino}-5,6,7,8- |
| | tetrahydronaphthalen-2-yl]oxy} acetate hydrochloride |
| Arbutamine | 4-(1-hydroxy-2-((4-(4-hydroxyphenyl)butyl)amino)ethyl)-1,2-Benzenediol |
| Arformoterol | (−)-Formoterol; (R,R)-Formoterol; arformoterol tartrate; N-(2-hydroxy-5-((1R)- |
| | 1-hydroxy-2-(((1R)-2-(4-methoxyphenyl)-1-methylethyl)amino)ethyl)phenyl)- |
| | formamide |
| ASF-1020 | WO 2006/108424 |
| AZ-40140 | SB-418790 |
| AZD-3199 | |
| Bambuterol | 5-(2-(tert-butylamino)-1-hydroxyethyl)-3-phenylene bis(dimethylcarbamate); |
| | bambuterol hydrochloride |
| Bedoradrine | bedoradrine sulfate; Bis (2-(((7S)-7-(((2R)-2-hydroxy-2-(4-hydroxy-3-(2- |
| | hydroxyethyl)phenyl)ethyl)amino)-5,6,7,8-tetrahydronaphthalen-2-yl)oxy)-N,N- |
| | dimethylacetamide) sulfate |
| BI-1744-CL | |
| Bitolterol | bitolterol mesylate; 4-(2-(tert-Butylamino)-1-hydroxyethyl)-o-phenylene |
| | di-p-toluate methanesulfonate |
| BMS-187257 | U.S. Pat. No. 5,321,036; Bioorganic & Medicinal Chemistry Letters |
| | 6(19): 2253-2258 (1996) |
| BMS-196085 | Bioorganic & Medicinal Chemistry Letters 11(23): 3041-3044 (2001). |
| BMS-210285 | 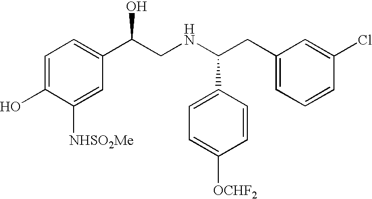 |
| BRL-26830A | methyl 4-(2-((2-hydroxy-2-phenethyl)amino)propyl)benzoate-2-butanedioate |
| BRL-35135 | EP 23385 |
| BRL-37344 | (4-(2-((2-(3-chlorophenyl)-2-hydroxyethyl)amino)propyl)phenoxy)acetic acid |
| Broxaterol | (+/−)-3-Bromo-alpha-((tert-butylamino)methyl)-5-isoxazolemethanol |
| Carmoterol | |
| Carvedilol | |
| CL-316243 | disodium (R,R)-5-(2-[{2-(3-chlorophenyl)-2-hydroxyethyl}-amino]propyl)- |
| | 1,3-benzodioxole-2,2,Dicarboxylate U.S. Pat. No. 5,061,727 |
| Clenbuterol | 4-amino-3,5-dichloro-alpha-(((1,1dimethylethyl)amino)methyl)-Benzenemethanol |
| CP-114271 | UL-TG-307 |
| Ephedrine | ephedrine hydrochloride; ephedrine sulfate; L(−)-Ephedrine |
| Fenoterol | Fenoterol Hydrobromide; Fenoterol Hydrochloride; Fenoterol + Ipratropium |
| | (Duovent or Berodual N) |
| Formoterol | Eformoterol, budesonide + formoterol; formoterol fumarate; fluticasone + |
| | formoterol; beclomethasone dipropionate + formoterol; ciclesonide + formoterol; |
| | mometasone + formoterol; Eformoterol fumarate dehydrate |
| FR-149175 | ethyl ((S)-8-((R)-2-(3-chlorophenyl)-2-hydroxy-ethylamino)-6,7,8,9- |
| | tetrahydro-5H-benzocyclohepton-2-yloxy)-acetate monohydrochloride |
| | monohydrate |
| FR-165914 | 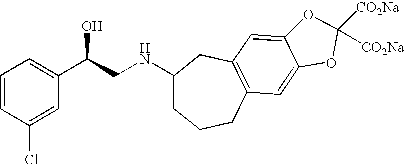 |
| GP-2-128 | 1-(3,4-dihydroxyphenyl)-2-(3-(4-carbamylphenyl)-1-methylpropylamino)ethanol; |
| | GP 2-128 (R-(R,R))-2,3-dihydroxybutanedioate (1:1); GP 2-128 monoacetate |
| GS-332 | sodium(2R)-(3-(3-(2-(3-chlorophenyl)-2-hydroxyethylamino)cyclohexyl) |
| | phenoxy)acetate |
| GSK 678007 | WO 03/066033 and WO 03/066036 |
| GSK-642444 | fluticasone furoate + GSK-642444 |
| GW-2696X | 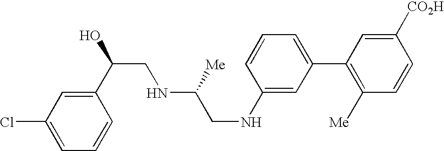 |
| Hexoprenaline | Hexoprenaline Sulfate |
| ICI-198157 | methyl (4-(2-((2-hydroxy-3-phenoxypropyl)amino)ethoxy)phenoxy) acetate |
| Indacaterol | glycopyrronium bromide + indacaterol; mometasone + indacaterol |
| Ipratropium | Ipratropium Bromide; Ipratropium Bromide, (endo,anti)-Isomer; Ipratropium |
| | Bromide, (exo,syn)-Isomer; (endo,syn)-(+−)-3-(3-Hydroxy-1-oxo-2- |
| | phenylpropoxy)-8-methyl-8-(1-methylethyl)-8-azoniabicyclo(3.2.1)octane; |
| Isoetharine | Isoetharine Mesylate; 4-(1-hydroxy-2-((1-methylethyl)amino)butyl)-1,2- |
| | Benzenediol |
| Isoproterenol | DL-Isoprenaline hydrochloride; DL-Isoproterenol hydrochloride; Isoproterenol |
| | Sulfate; 4-(1-Hydroxy-2-((1-methylethyl)amino)ethyl)-1,2-benzenediol; |
| | isoproterenol hydrochloride + phenylephrine bitartrate |
| KUL-1248 | 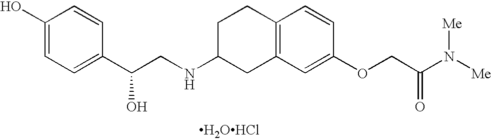 |
| KUL-7211 | 2-(4-(2-((2-hydroxy-2-(4-hydroxyphenyl)-1-methylethyl)amin)ethyl)phenyloxy) |
| | acetic acid |
| L-742791 | (S)-N-(4-[2-{(3-[4-hydroxyphenoxy]-2-hydroxypropyl)amino}ethyl]phenyl)- |
| | 4-iodobenzenesulfonamide |
| LAS-10097 | |
| Levalbuterol | controlled-release oral levalbuterol; R-salbutamol sulfate |
| LM-2616 | 4-(4-Methyl-1-piperazinyl)-2,7,9-trimethylpyrido(3′,2′:4,5)thieno(3,2- |
| | d)pyrimidine |
| MAP-0005 | Budesonide + formoterol |
| meluadrine | (−)-(R)-alpha-((tert-Butylamino)methyl)-2-chloro-4-hydroxybenzyl alcohol; |
| | meluadrine tartrate |
| Metaproterenol | (RS)-1-(3,5-Dihydroxyphenyl)-2-isopropylaminoethanol; metaproterenol-3- |
| | O-sulfate |
| Milveterol | fluticasone furoate + milveterol |
| Mirabegron | 2-(2-amino-1,3-thiazol-4-yl)-N-[4-(2-{[(2R)-2-hydroxy-2-phenylethyl]amino} |
| | ethyl)phenyl]acetamide |
| N-5984 | 6-(2-(R)-((2-(R)-(3-chlorophenyl)-2-hydroxyethyl)amino)propyl)-2,3- |
| | dihydro-1,4-benzodioxine-2-(R)-carboxylic acid |
| NCX-950 | α′-[[(1,1-dimethylethy)amino]methyl]-4-hydroxy-1,3-benzenedimethanol nitrate |
| Nylidrin | Buphenine Hydrochloride; p-Hydroxy-N-(1-methyl-3-phenylpropyl)norephedrine |
| PF-610355 | PF-610355 + Salmeterol |
| Picumeterol | picumeterol fumarate; (R)-4-Amino-3,5-dichloro-alpha-(((6-(2-(2-pyridinyl) |
| | ethoxy)hexyl)amino)methyl)benzenemethanol |
| Pirbuterol | pirbuterol acetate; pirbuterol dihydrochloride; pirbuterol sulfate; 2- |
| | hydroxymethyl-3-hydroxy-6-(1-hydroxy-2-tert-butylamino ethyl)pyridine, |
| | dihydrochloride; |
| Procaterol | (R,S)-(+−)-8-Hydroxy-5-(1-hydroxy-2-((1-methylethyl)amino)butyl)- |
| | 2(1H)-quinolinone; Procaterol Monohydrochloride; Procaterol |
| | Monohydrochloride, (R,R)-(+)-Isomer; Procaterol Monohydrochloride, |
| | (R,R)-(+−)-Isomer; Procaterol Monohydrochloride, (R,R)-(−)-Isomer; |
| | Procaterol Monohydrochloride, (R,S)-(+)-Isomer; Procaterol |
| | Monohydrochloride, (R,S)-(−)-Isomer; Procaterol, (R,R)-(+−)-Isomer; |
| | Procaterol, (R,S)-(−)-Isomer |
| Rafabegron | (3-{(2R)-2-((2R)-2-(3-Chlorophenyl)-2-hydroxyethylamino)propyl}-1H- |
| | indol-7-yloxy)acetic acid |
| Reproterol | reproterol monohydrochloride; reproterol, (−)-isomer; Reproterol + |
| | cromoglycate |
| Rimiterol | alpha-(3,4-Dihydroxyphenyl)-2-piperidinemethanol |
| Ritodrine | Ritodrine Hydrochloride |
| RP-58802B | 2-(3-(1-Benzimidazolyl)-1-methylpropylamino)-1-(4-hydroxy-3-methoxyphenyl) |
| | ethanol; alpha-(((3-(1-Benzimidazolyl)-1-methylpropyl)amino)methyl) |
| | vanillyl Alcohol; RP 58802B dihydrochloride; RP 58802B, (R,R)-(+−)- |
| | isomer; RP 58802B, (R,S)-(+−)-isomer; RP 58802B, (R-(R,R))-isomer; |
| | RP 58802B, (R-(R,S))-isomer; RP 58802B, (S-(R,R))-isomer; |
| | RP 58802B, (S-(R,S))-isomer; |
| S-1319 | 4-hydroxy-7-(1-(1-hydroxy-2-methylamino)ethyl)-1,3-benzothiazole- |
| | 2(3H)-one |
| Salbutamol | albuterol; albuterol sulfate; ipratropium + salbutamol (Combivent and |
| | Duoneb), Merck KGaA; ipratropium bromide + salbutamol sulfate |
| Salmeterol | fluticasone propionate + salmeterol; R-salmeterol |
| SAR-150640 | ethyl 4-(4-((2-hydroxy-3-(4-hydroxy-3-((methylsulfonyl)amino)phenoxy) |
| | propyl)amino)cyclohexyl)benzoate; ethyl-4-{trans-4-[((2S)-2-hydroxy- |
| | 3-{4-hydroxy-3[(methylsulfonyl)amino]phenoxy}propyl)amino]cyclohexyl} |
| | benzoate hydrochloride |
| SB-220646 | Journal of Pharmacology and Experimental Therapeutics, 285: |
| | 1084-1095 (1998) |
| SB-226552 | Journal of Pharmacology and Experimental Therapeutics, 285: |
| | 1084-1095 (1998) |
| SB-236923 | Journal of Pharmacology and Experimental Therapeutics, 285: |
| | 1084-1095 (1998) |
| SB-251023 | (4-[1-{2-(S)-hydroxy-3-(4-hydroxyphenoxy)-propylamino} |
| | cyclopentylmethyl]phenoxymethyl)phenylphosphonic acid lithium salt |
| Sibenadet | sibenadet hydrochloride |
| SM-11044 | 3-(3,4-dihydroxyphenyl)-N-(3-(4-fluorophenyl)propyl)serine pyrrolidine |
| | amide hydrobromide; (R-(R,S))-1-(3-(3,4-dihydroxyphenyl)-2-((3-(4- |
| | fluorophenyl)propyl)amino)-3-hydroxy-1-oxopropyl)-, |
| | monohydrobromidepyrrolidine, |
| Solabegron | 3′-((2-((2-(3-chlorophenyl)-2-hydroxyethyl)amino)ethyl)amino)- |
| | (1,1′-biphenyl)-3-carboxylic acid |
| Sotalol | beta-Cardone; Betapace; Betapace AF; Hydrochloride, Sotalol; MJ 1999; |
| hydrochloride | MJ-1999; MJ1999; Monohydrochloride, Sotalol; Sotalol Hydrochloride; |
| | Sotalol Monohydrochloride; Sotalex; Sotacor |
| SR-59062A | Bioorganic & Medicinal Chemistry Letters Volume 4, Issue 16, 25 August 1994, |
| | Pages 1921-1924 |
| SR-59104A | N-[(2R)-(6-hydroxy-1,2,3,4-tetrahydronaphth-2-yl)methyl]-(2R)-2-hydroxy- |
| | 2-(3-chlorophenyl)ethanamine |
| SR-59119A | N-[(2R)-(7-methoxy-1,2,3,4-tetrahydronaphth-2-yl)methyl]-(2R)-2-hydroxy- |
| | 2-(3-chlorophenyl)ethanamine |
| SWR-0342-SA | (S)-(Z)-[4-[[1-[2-[(2-hydroxy-3-phenoxypropyl)]amino]ethyl]-1- |
| | propenyl]phenoxy]acetic acid ethanedioic acid |
| T-0509 | 4-(2-((2-(3,4-dimethoxyphenyl)ethyl)amino)-1-hydroxyethyl)-1,2-benzenediol; |
| | 1-(3,4-dimethoxyphenethyl amino)-2-(3,4-dihydroxyphenyl)ethanol; |
| | RP333, (R)-isomer; RP333, dihydrochloride, (+)-isomer; RP333, hydrochloride |
| Talibegron | 4-[2-[[(2R)-2-hydroxy-2-phenylethyl]amino]ethoxy]-benzeneacetic acid, |
| | hydrochloride |
| Terbutaline | Terbutaline Sulfate |
| Tulobuterol | 1-(o-chlorophenyl)-2-tert-butylaminoethanol; tulobuterol hydrochloride; |
| | alpha-((tert-butylamino)methyl)-o-chlorobenzyl alcohol |
| UD-CG-212 | 3(2H)-Pyridazinone, 4,5-dihydro-6-(2-(4-hydroxyphenyl)-1H- |
| | benzimidazol-5-yl)-5-methyl-, monohydrochloride; |
| UK-503590 | |
| UL-TG-307 | 4-(2-((2-hydroxy-2-(2-(trifluoromethyl)-4-thiazolyl)ethyl)-amino)propyl) |
| | phenoxy-acetic acid |
| Xamoterol | Xamoterol Fumarate; Xamoterol Hemifumarate; Xamoterol Maleate |
| | (2:1); Xamoterol Monohydrobromide; Xamoterol Monohydrochloride; |
| | Xamoterol, (S)-Isomer; |
| ZD-7114 | EP 473 285 |

Preferred BAR agonists include arbutaline, arfomoterol, bambuterol, bitolterol, broxaterol, clenbuterol, fenoterol, formoterol, hexoprenaline, indacaterol, isoetharine, isoproterenol, levalbuterol, meluadrine, metaproterenol, nylidrin, picumeterol, pirbuterol, procaterol, reproterol, rimiterol, ritodrine, salbutamol, salmeterol, tulobuterol, terbutaline, and xamoterol.

Additional BAR agonists for use in the invention are shown in Table 2.

TABLE 2

159802 (GSK-159802)
ZD-9989
SWR-0335
MN-246
LY-362884
L-751250
KUR-1247
KI-03219
KTO-7924
GSK-597901
GRC-1087
CL-314698
AZD-3199
799943 (GSK-799943)
961081 (GSK-961081)
AR-C-89855
anti-obesity therapy, Nisshin Flour
asthma therapy, Cue Biotech
asthma therapy, Selectus
beta 2 adrenoceptor agonists (asthma/COPD),
Novartis
beta 3 adrenoceptor agonists (obesity/diabetes),
Wyeth
beta 3 adrenoceptor agonists, University of
Tennessee
beta2-agonist, Byk Nederland
beta-3 adrenergic (human) receptor, OncoPharm
beta-3 adrenoceptor agonist, MDI
beta-3 adrenoceptor agonist, Pfizer
beta-3 adrenoceptor agonists (obesity), Merck &
Co
beta-3 adrenoceptor agonists, Lilly
beta-3 agonist, Bayer
beta-3 agonists, Fourier de Grenoble
EPI-12323 combination therapy (asthma/COPD),
EpiGenesis
long acting beta agonists (asthma/COPD),
Sepracor
short acting beta agonists (solution, asthma),
AstraZeneca
salbutamol esters, Cardiff University
SR-58878

In certain embodiments, isoproterenol is not employed.

### A2A Receptor Agonists

Exemplary A2A receptor agonists for use in the invention are shown in Table 3.

TABLE 3

| Compound | Synonym |
|---|---|
| FK 453 | (+)-(R)-[(E)-3-(2-phenylpyrazolo[1,5-a]pyridin-3-yl)acryloyl]- |
| | 2-piperidine ethanol |
| N 0861 | (+−)-N6-endonorbornan-2-yl-9-methyladenine |
| CVT 3619 | (2-{6-[((1R,2R)-2-hydroxycyclopentyl)amino]purin- |
| | 9-yl}(4S,5S,2R,3R)-5-[(2-fluorophenylthio) methyl]oxolane- |
| | 3,4-diol) |
| T 62 | (2-amino-4,5,6,7-tetrahydrobenzo[b]thiophen-3-yl)- |
| | (4-chlorophenyl)-methanone |
| PD 81723 | (2-Amino-4,5-dimethyl-3-thienyl)-[3- |
| | (trifluoromethyl)phenyl]methanone |
| CP 608039 | (2S,3S,4R,5R)-3-amino-5-{6-[5-chloro-2-(3- |
| | methyl-isoxazol-5-ylmethoxy)-benzylamino]-purin- |
| | 9-yl}-4-hydroxy-tetrahydro-furan-2-carboxylic acid |
| | methylamide |
| CVT 3033 | (4S,2R,3R,5R)-2-[6-amino-2-(1-pentylpyrazol-4- |
| | yl)purin-9-yl]-5-(-hydroxymethyl)oxolane-3,4-diol |
| FK 352 | (E)-(R)-1-[3-(2-phenylpyrazolo[1,5-a]pyridin-3- |
| | yl)acryloyl]pyperidin-2-ylacetic acid |
| KF 21213 | (E)-8-(2,3-dimethyl-4-methoxystyryl)-1,3,7- |
| | trimethylxanthine |
| KE 17837 | (E)-8-(3,4-dimethoxystyryl)-1,3-dipropyl-7- |
| | methylxanthine |
| MDL 102503 | (R)-3,7-dihydro-8-(1-methyl-2-phenylethyl)-1,3- |
| | dipropyl-1H-purine-2,6-dione |
| Apaxifylline | (S)-3,7-dihydro-8-(3-oxocyclopentyl)-1,3-dipropyl- |
| | 1H-purine-2,6-dione |
| CVT 2759 | [(5-{6-[((3R)oxolan-3-yl)amino]purin-9- |
| | yl}(3S,2R,4R,5R)-3,4-dihydroxyoxolan-2- |
| | yl)methoxy]-N-methylcarboxamide |
| DAX | 1,3-diallyl-8-cyclohexylxanthine |
| BG 9928 | 1,3-dipropyl-8-[1-(4-propionate)-bicyclo- |
| | [2,2,2]octyl]xanthine |
| CPX | 1,3-dipropyl-8-cyclopentylxanthine |
| BN 063 | 1-cyclopropylisoguanosine |
| AMP 579 | 1S-[1a,2b,3b,4a(S*)]-4-[7-[[1-[(3-chloro-2- |
| | thienyl)methylpropyl]propyl-amino]-3H- |
| | imidazo[4,5-b] pyridyl-3-yl]-N-ethyl-2,3- |
| | dihydroxycyclopentane carboxamide |
| Binodenoson (MRE-0470) | 2-((cyclohexylmethylene)hydrazino)-Adenosine |
| HEMADO | 2-(1-hexynyl)-N-methyladenosine |
| YT 146 | 2-(1-octynyl) adenosine |
| Regadenoson (CVT 3146) | 2-(4-((methylamino)carbonyl)-1H-pyrazol-1-yl)- |
| | Adenosine |
| CGS 21680 | 2-(4-(2-carboxyethyl)phenethylamino)-5′-N- |
| | ethylcarboxamidoadenosine |
| APEC | 2-[(2-aminoethyl-aminocarbonylethyl)phenylethylamino]- |
| | 5′-N-ethyl-carboxamidoadenosine |
| MRE 0094 | 2-[2-(4-chlorophenyl)ethoxy]adenosine |
| 2-Cl-IB-MECA | 2-chloro-N⁶-(3-iodobenzyl)-5′-N- |
| | methylcarboxamidoadenosine |
| CCPA | 2-chloro-N⁶-cyclopentyladenosine |
| CV 1808 | 2-phenylaminoadenosine |
| MDL 102234 | 3,7-dihydro-8-(1-phenylpropyl)-1,3-dipropyl-1H- |
| | purine-2,6-dione |
| SF 349 | 3-acetyl-7-methyl-7,8-dihydro-2,5(1H,6H)quinolinone |
| CVT 6883 | 3-ethyl-1-propyl-8-[1-(3-trifluoromethylbenzyl)-1H- |
| | pyrazol-4-yl]-3,7-dihydropurine-2,6-dione |
| UR 7247 | 3-iso-propyl-5-([2′-{1H}-tetrazol-5-yl-1,1′- |
| | biphenyl-4-yl]methyl)-1Hpyrazole-4-carboxylic |
| | acid |
| ZM 241385 | 4-(2-[7-amino-2-(2-furyl)[1,2,4]-triazolo[2,3- |
| | a][1,3,5]triazin-5-yl amino]ethyl)phenol |
| IRFI 165 | 4-Cyclopentylamino-1.-methylimidazo[1,2- |
| | alquinoxaline |
| FK 838 | 6-oxo-3-(2-phenylpyrazolo [1,5-a] pyridin-3-yl)- |
| | 1(6H)-pyridazinebutanoic acid |
| KF 20274 | 7,8-dihydro-8-ethyl-2-(3-noradamantyl)-4-propyl- |
| | 1H-imidazo(2,1-j)purin-5(4H)-one |
| Midaxifylline | 8-(1-Aminocyclopentyl)-3,7-dihydro-1,3-dipropyl- |
| | (1H)-purine-2,6-dione hydrochloride |
| KFM 19 | 8-(3oxocyclopentyl)-1,3-dipropyl-7H-purine-2,6- |
| | dione |
| KW 3902 | 8-(noradamantan-3-yl)-1,3-dipropylxanthine |
| Naxifylline | 8-[(1S,2R,4S,5S,6S)-3-oxatricyclo[3.2.1.02,4]oct-6- |
| | yl]-1,3-dipropyl-3,7-dihydro-1H-purine-2,6-dione |
| DPCPX | 8-cyclopentyl-1,3-dipropylxanthine |
| MDL 201449 | 9-[(1R,3R)-trans-cyclopentan-3-ol]adenine |
| CPC 405 | 9′-chloro-EHNA |
| CPC 402 | 9′-hydroxy-EHNA |
| CPC 406 | 9′-phthalimido-EHNA |
| WRC 0571 | C⁸-(N-methylisopropyl)-amino-N⁶(5′-endohydroxy)- |
| | endonorbornan-2-yl-9-methyladenine |
| HE-NECA | hexynyladenosine-5′-N-ethylcarboxamide |
| GR 79236 | N-((1S,trans)-2-hydroxycyclopentyl)adenosine |
| Metrifudil | N-((2-methylphenyl)methyl)adenosine |
| R-PIA | N-(1-methyl-2-phenylethyl)adenosine |
| ADAC | N-(4-(2-((4-(2-((2-aminoethyl)amino)-2- |
| | oxoethyl)phenyl)amino)-2-oxoethyl)phenyl)- |
| | Adenosine |
| DPMA | N⁶-(2-(3,5-dimethoxyphenyl)-2-(2- |
| | methylphenyl)ethyl)adenosine |
| IB-MECA | N⁶-(3-iodobenzyl)-5′-N- |
| | methylcarboxamidoadenosine |
| I-AB-MECA | N⁶-(4-amino-3-iodophenyl)methyl-5′-N- |
| | methylcarboxamidoadenosine |
| WRC 0342 | N⁶-(5′-endohydroxy)-endonorbornan-2-yl-9- |
| | methyladenine |
| HPIA | N⁶-(R-4-hydroxyphenylisopropyl) adenosine |
| CGS 24012 | N6-2-(3,5-dimethoxyphenyl)-2-(2-methylphenyl)- |
| | ethyl adenosine |
| SDZ WAG 994 | N⁶-cyclohexyl-2′-O-methyladenosine |
| CHA | N⁶-cyclohexyladenosine |
| TCPA | N⁶-cyclopentyl-2-(3-phenylaminocarbonyltriazene- |
| | 1-yl)adenosine |
| N 0840 | N6-cyclopentyl-9-methyladenine |
| CPA | N⁶-cyclopentyladenosine |
| NECA | N-ethylcarboxamidoadenosine |
| (S)-ENBA | S—N⁶-(2-endo-norbornyl)adenosine |
| Apadenoson | trans-4-(3-(6-amino-9-(N-ethyl-.beta.-D- |
| | ribofuranuronamidosyl)-9H-purin-2-yl)-2-propynyl)- |
| | Cyclohexanecarboxylic acid methyl ester |
| CDS 096370 | U.S. Pat. No. 6,800,633 |
| ATL-313 | 4-{3-[6-amino-9-(5-cyclopropylcarbamoyl-3,4- |
| | dihydroxytetrahydrofuran-2-yl)-9H-purin-2-yl]prop- |
| | 2-ynyl}piperidine-1-carboxylic acid methyl ester |
| ATL-193 | acetic acid 4-{3-[6-amino-9-(5-ethylcarbamoyl-3,4- |
| | dihydroxy-tetrahydro-furan-2-yl)-9H-purin-2-yl]- |
| | prop-2-ynyl}-cyclohexylmethyl ester |
| ATL2037 | 5-{6-amino-2-[3-(4-hydroxymethyl-cyclohexyl)- |
| | prop-1-ynyl]-purin-9-yl}-3,4-dihydroxy-tetrahydro- |
| | furan-2-carboxylic acid ethylamide; BW-1433, 8-(4- |
| | carboxyethenylphenyl)-1,3-dipropylxanthine |

Additional A2A receptor agonists are described or claimed in US Patent Application Publication Nos. 20020082240, 20030186926, 20050261236, 2006040888, 20060040889, 20060217343, 20070232559, 20080262001, 20080064653, and 20080312160 and U.S. Pat. Nos. 5,877,180, 6,448,235, 7,214,665, 7,217,702, 7,226,913, 7,396,825, and 7,442,687.

Additional adenosine receptor agonists are shown in Table 4.

TABLE 4

| | | |
|---|---|---|
| 3′-Aminoadenosine-5′- | A15PROH | Adenosine |
| uronamides | | |
| Adenosine amine congener | Adenosine hemisulfate salt | BAY 68-4986 |
| solid | | |
| BIIB014 | BVT 115959 | CF 402 |
| CVT 2501 | DTI 0017 | GP 3367 |
| GP 3449 | GP 4012 | GR 190178 |
| GW 328267 | GW 493838 | Istradefylline |
| KF 17838 | M 216765 | MDL 101483 |
| NipentExtra | NNC 210113 | NNC 210136 |
| NNC 210147 | NNC 901515 | OSIC 113760 |
| SCH 420814 | SCH 442416 | SCH 59761 |
| Selodenoson (DTI-0009) | SLV 320 | SSR 161421 |
| SYN 115 | Tecadenoson (CVT-510) | UK 432097 |
| UP 20256 | WRC 0542 | Y 341 |

Other adenosine receptor agonists are those described or claimed in Gao et al., JPET, 298: 209-218 (2001); U.S. Pat. Nos. 5,278,150, 5,424,297, 5,877,180, 6,232,297, 6,448,235, 6,514,949, 6,670,334, and 7,214,665; U.S. Patent Application Publication No. 20050261236, and International Publication Nos. WO98/08855, WO99/34804, WO2006/015357, WO2005/107463, WO03/029264, WO2006/023272, WO00/78774, WO2006/028618, WO03/086408, and WO2005/097140, incorporated herein by reference.

Preferred A2A agonists include adenosine, regadenoson, apadenoson, sonedenoson, MRE-0094, BVT-115959, UK-432097, acadesine, tocladesine, CGS-21680C and CGS-21680, spongosine, binodenoson, HE-NECA, IB-MECA, CI-IB-MECA, NECA, ATL-313, ATL-1222, and DPMA.

### PDE Inhibitors

Exemplary PDE inhibitors for use in the invention are shown in Table 5.

TABLE 5
PDE Inhibitors

| Compound | Synonym | PDE Activity |
|---|---|---|
| 349U85 | 6-piperidino-2(1H)-quinolinone | 3 |
| Adibendan | 5,7-dihydro-7,7-dimethyl-2-(4-pyridinyl)- | 3 |
| | pyrrolo(2,3-f)benzimidazol-6(1H)-one | |
| Amlexanox | 2-amino-7-isopropyl-5-oxo-5H- | 3, 4 |
| | [1]benzopyrano[2,3-b]pyridine-3-carboxylic | |
| | acid (U.S. Pat. No. 4,143,042) | |
| Amrinone | 5-amino-(3,4′-bipyridin)-6(1H)-one | 3, 4 |
| Anagrelide | U.S. Pat. No. 3,932,407 | 3, 4 |
| AP 155 | 2-(1-piperazinyl)-4H-pyrido[1,2-a]pyrimidin- | 4 |
| | 4-one | |
| AR 12456 | CAS Reg. No. 100557-06-0 | 4 |
| Arofylline | 3-(4-chlorophenyl)-3,7-dihydro-1-propyl-1H- | 4 |
| | purine-2,6-dione | |
| Ataquimast | 1-ethyl-3-(methylamino)-2(1H)-quinoxalinone | 3 |
| Atizoram | tetrahydro-5-[4-methoxy-3-[(1S,2S,4R)-2- | 4 |
| | norbornyloxy]phenyl]- | |
| | 2(1H)-pyrimidinone | |
| ATZ 1993 | 3-carboxy-4,5-dihydro-1-[1-(3- | |
| | ethoxyphenyl)propyl]-7-(5- | |
| | pyrimidinyl)methoxy-[1H]-benz[g]indazole | |
| | (Teikoku Hormone) | |
| Avanafil | 4-{[(3-chloro-4- | 5 |
| | methoxyphenyl)methyl]amino}-2-[(2S)-2- | |
| | (hydroxymethyl)pyrrolidin-1-yl]-N- | |
| | (pyrimidin-2-ylmethyl)pyrimidine- | |
| | 5-carboxamide | |
| AVE 8112 | | 4 |
| AWD 12171 | | 5 |
| AWD 12187 | | 7 |
| AWD 12250 | | 5 |
| AWD 12343 | | 4 |
| BAY 38-3045 | | 1 |
| BAY 60-7550 (Alexis | 2-(3,4-dimethoxybenzyl)-7-[(1R)-1-[(1R)-1- | 2 |
| Biochemicals) | hydroxyethyl]-4-phenylbutyl]-5- | |
| | methylimidazo[5,1-f][1,2,4]triazin-4(3H)-one | |
| BBB 022 | | 4 |
| Bemarinone | 5,6-dimethoxy-4-methyl-2(1H)-quinazolinone | 3 |
| Bemoradan | 6-(3,4-dihydo-3-oxo-1,4(2H)-benzoxazin-7- | 3 |
| | yl)-2,3,4,5-tetrahydro-5-methylpyridazin-3- | |
| | one | |
| Benafentrine | (6-(p-acetamidophenyl)-1,2,3,4,4a,10b- | 3, 4 |
| | hexahydro-8,9-dimethoxy-2-methyl- | |
| | benzo[c][1,6]naphthyridine | |
| BMY 20844 | 1,3-dihydro-7,8-dimethyl-2H-imidazo[4,5- | 4 |
| | b]quinolin-2-one | |
| BMY 21190 | | 4 |
| BMY 43351 | 1-(cyclohexylmethyl)-4-(4-((2,3-dihydro-2- | 4 |
| | oxo-1H-imidazo(4,5-b)quinolin-7-yl)oxy)-1- | |
| | oxobutyl)-Piperazine | |
| BRL 50481 | 3-(N,N-dimethylsulfonamido)-4-methyl- | 7 |
| | nitrobenzene | |
| C 3885 | | 4 |
| Caffeine citrate | 2-hydroxypropane-1,2,3-tricarboxylic acid | 4 |
| CC 10004 | N-(2-((1S)-1-(3-ethoxy-4-methoxyphenyl)-2- | 4 |
| | (methylsulfonyl)ethyl)-2,3-dihydro-1,3-dioxo- | |
| | 1H-isoindol-4-yl)-acetamide | |
| CC 1088 | | 4 |
| CC 3052 | The Journal of Immunology, 1998, 161: 4236-4243 | 4 |
| CC 7085 | | 4 |
| CCT 62 | 6-[(3-methylene-2-oxo-5-phenyl-5- | 3 |
| | tetrahydrofuranyl)methoxy]quinolinone | |
| CDC 998 | | 4 |
| CDP 840 | 4-((2R)-2-(3-(cyclopentyloxy)-4- | 4 |
| | methoxyphenyl)-2-phenylethyl)-pyridine | |
| CGH 2466 | 2-amino-4-(3,4-dichlorophenyl)-5-pyridin-4- | 4 |
| | yl-thiazol | |
| CI 1018 | N-(3,4,6,7-tetrahydro-9-methyl-4-oxo-1- | 4 |
| | phenylpyrrolo(3,2,1-jk)(1,4)benzodiazepin-3- | |
| | yl)-4-pyridinecarboxamide | |
| CI 1044 | N-[9-amino-4-oxo-1-phenyl-3,4,6,7- | 4 |
| | tetrahydropyrrolo[3,2,1-jk][1,4]b- | |
| | enzodiazepin-3(R)-yl]pyridine-3-carboxamide | |
| CI 930 | 4,5-dihydro-6-[4-(1H-imidazol-1-yl)phenyl]- | 3 |
| | 5-methyl-3(2H)-pyridazinone | |
| Cilomilast (Ariflo ®) | 4-cyano-4-(3-cyclopentyloxy-4-methoxy- | 4 |
| | phenyl)cyclohexane-1-carboxylic acid (U.S. | |
| | Pat. No. 5,552,438) | |
| Cilostamide | N-cyclohexyl-4-((1,2-dihydro-2-oxo-6- | 3 |
| | quinolinyl)oxy)-N-methyl-butanamide | |
| Cilostazol | 6-[4-(1-cyclohexyl-1H-tetrazol-5-yl)butoxy]- | 3, 4 |
| | 3,4-dihydro-2(1H)-quinolinone (U.S. Pat. | |
| | No. 4,277,479) | |
| Cipamfylline | 8-amino-1,3-bis(cyclopropylmethyl)-3,7- | 4 |
| | dihydro-1H-purine-2,6-dione | |
| CK 3197 | 2H-imidazol-2-one, 1-benzoyl-5-(4-(4,5- | |
| | dihydro-2-methyl-1H-imidazol-1-yl)benzoyl)- | |
| | 4-ethyl-1,3-dihydro | |
| CP 146523 | 4′-methoxy-3-methyl-3′-(5-phenyl- | 4 |
| | pentyloxy)-biphenyl-4-carboxylic acid | |
| CP 220629 | 1-cyclopentyl-3-ethyl-6-(2-methylphenyl)-7- | 4 |
| | oxo-4,5,6,7-tetrahydro-1H-pyrazolo[3,4- | |
| | c]pyridine | |
| CP 248 | (Z)-5-fluoro-2-methyl-1-[p- | 2 |
| | (methylsulfonyl)benzylidene]indene-3-acetic | |
| | acid | |
| CP 293121 | (S)-3-(3-cyclopentyloxy-4-methoxy)phenyl-2- | 4 |
| | isoxazoline-5-hydroxamic acid | |
| CP 353164 | 5-(3-cyclopentyloxy-4-methoxy-phenyl)- | 4 |
| | pyridine-2-carboxylic acid amide | |
| CT 2820 | | |
| D 22888 | 8-methoxy-5-N-propyl-3-methyl-1-ethyl- | 4 |
| | imidazo [1,5-a]-pyrido [3,2-e]-pyrazinone | |
| D 4418 | N-(2,5-dichloro-3-pyridinyl)-8-methoxy-5- | 4 |
| | quinolinecarboxamide | |
| Dasantafil | 7-(3-bromo-4-methoxyphenylmethyl)-1-ethyl- | 5 |
| | 8-{[(1R,2R)-2-hydroxycyclopentyl] = amino}- | |
| | 3-(2-hydroxyethyl)-3,7-dihydro-1H- | |
| | purine-2,6-dione | |
| Dipyridamole | 2-{[9-(bis(2-hydroxyethyl)amino)-2,7-bis(1- | 5, 6, 7, 8, 10, |
| | piperidyl)-3,5,8,10-tetrazabicyclo[4.4.0]deca- | 11 |
| | 2,4,7,9,11-pentaen-4-yl]-(2- | |
| | hydroxyethyl)amino}ethanol | |
| DN 9693 | 1,5-dihydro-7-(1-piperidinyl)-imidazo[2,1- | 4 |
| | b]quinazolin-2(3H)-one dihydrochloride | |
| | hydrate | |
| Doxofylline | 7-(1,3-dioxolan-2-ylmethyl)-1,3-dimethyl-3,7- | 4 |
| | dihydro-1H-purine-2,6-dione (U.S. Pat. No. | |
| | 4,187,308) | |
| E 4010 | 4-(3-chloro-4-metoxybenzyl)amino-1-(4- | 5 |
| | hydroxypiperidino)-6-phthalazinecarbonitrile | |
| | monohydrochloride | |
| E 4021 | sodium 1-[6-chloro-4-(3,4- | 4, 5 |
| | methylenedioxybenzyl)aminoquinazolin-2- | |
| | yl]piperidine-4-carboxylate sesquihydrate | |
| EHNA | erythro-9-(2-hydroxy-3-nonyl)adenine | 2, 3, 4 |
| EHT 0202 | 3,7-dimethyl-1-(5-oxohexyl)purine-2,6-dione | 4 |
| ELB 353 | | 4 |
| EMD 53998 | 5-(1-(3,4-dimethoxybenzoyl)-1,2,3,4- | 3 |
| | tetrahydro-6-quinolyl)-6-methyl-3,6-dihydro- | |
| | 2H-1,3,4-thiadiazin-2-one | |
| EMD 57033 | (+)-5-[1-(3,4-dimethoxybenzoyl)-3,4-dihydro- | 3 |
| | 2H-quinolin-6-yl]-6-methyl-3,6-dihydro-1,3,4- | |
| | thiadiazin-2-one | |
| EMD 57439 | (−)-5-[1-(3,4-dimethoxybenzoyl)-3,4-dihydro- | 3 |
| | 2H-quinolin-6-yl]-6-methyl-3,6-dihydro-1,3,4- | |
| | thiadiazin-2-one | |
| EMD 82639 | | 5 |
| EMR 62203 | | 5 |
| Enoximone | U.S. Pat. No. 4,405,635 | 3 |
| Enprofylline | 3-propyl xanthine | 4 |
| ER 017996 | 4-((3,4-(methylenedioxy)benzyl)amino)-6,7,8- | |
| | trimethoxyquinazoline | |
| Etazolate | 1-ethyl-4-((1-methylethylidene)hydrazino)-1h- | 4 |
| | pyrazolo(3,4-b) pyridine-5-carboxylic acid | |
| Exisulind | (1Z)-5-fluoro-2-methyl-1-[[4- | 2, 5 |
| | (methylsulfonyl)phenyl]methylene]-1H- | |
| | indene-3-acetic acid | |
| Filaminast | (1E)-1-(3-(cyclopentyloxy)-4- | 4, 7 |
| | methoxyphenyl)-ethanone O- | |
| | (aminocarbonyl)oxime | |
| FR 226807 | N-(3,4-dimethoxybenzyl)-2-{[(1R)-2- | 5 |
| | hydroxy-1-methylethyl]amino}-5- | |
| | nitrobenzamide | |
| FR 229934 | | 5 |
| GI 104313 | 6-{4-[N-[-2-[3-(2-cyanophenoxy)-2- | 3 |
| | hydroxypropylamino]-2- | |
| | methylpropyl]carbamoylmethoxy-3- | |
| | chlorophenyl]}-4,5-dihydro-3(2H)pyridazinone | |
| GRC 3015 | | 4 |
| GSK 256066 | | 4 |
| GW 3600 | (7aS,7R)-7-(3-cyclopentyloxy-4- | 4 |
| | methoxyphenyl)-7a-methyl-2,5,6,7,7a-penta- | |
| | hydro-2-azapyrrolizin-3-one | |
| GW 842470 | N-(3,5-dichloro-4-pyridinyl)-1-((4- | 4 |
| | fluorophenyl)methyl)-5-hydroxy-α-oxo-1H- | |
| | indole-3-acetamide | |
| Helenalin | CAS Reg. No. 6754-13-8 | 5 |
| Hydroxypumafentrine | | 4 |
| IBMX | 3-isobutyl-1-methylxanthine | 3, 4, 5 |
| Ibudilast | 1-(2-isopropyl-pyrazolo[1,5-a]pyridine-3-yl)- | Not selective |
| | 2-methylpropan-1-one (U.S. Pat. No. | |
| | 3,850,941) | |
| IC 485 | | 4 |
| IPL 455903 | (3S,5S)-5-(3-cyclopentyloxy-4-methoxy- | 4 |
| | phenyl)-3-(3-methyl-benzyl)-piperidin-2-one | |
| Isbufylline | 1,3-dimethyl-7-isobutylxanthine | 4 |
| KF 17625 | 5-phenyl-1H-imidazo(4,5-c)(1,8)naphthyridin- | 4 |
| | 4(5H)-one | |
| KF 19514 | 5-phenyl-3-(3-pyridil) methyl-3H- | 1, 4 |
| | imidazo[4,5-c][1,8]naphthyridin-4(5H)-one | |
| KF 31327 | 3-ethyl-8-[2-[4-(hydroxymethyl)piperidin-1- | 5 |
| | yl]benzylamino]-2,3-dihydro-1H-imidazo[4,5- | |
| | g]quinazoline-2-thione | |
| Ks-505a | 1-carboxy- | 1 |
| | 2,3,4,4a,4b,5,6,6a,6b,7,8,8a,8b,9,10,10a,- | |
| | 14,16,17,17a,17b,18,19,19a,19b,- | |
| | 20,21,21a,21b,22,23,23a-dotriacontahydro-14- | |
| | hydroxy-8a,10a-bis(hydroxymethyl)-14-(3- | |
| | methoxy-3-oxopropyl)-1,4,4a,- | |
| | 6,6a,17b,19b,21b-octamethyl beta-D- | |
| | glucopyranosiduronic acid | |
| KT 734 | | 5 |
| KW 4490 | | 4 |
| L 686398 | 9-[1,S,2R)-2-fluoro-1-methylpropyl]-2- | 3, 4 |
| | methoxy-6-(1-piperazinyl]-purine | |
| | hydrochloride | |
| L 826141 | 4-{2-(3,4-bis-difluromethoxyphenyl)-2-{4- | 4 |
| | (1,1,1,3,3,3-hexafluoro-2-hydroxypropan-2- | |
| | yl)-phenyl]-ethyl}-3-methylpyridine-1-oxide | |
| L 869298 | (+)-1\|(S)-(+)-3-{2-[(3-cyclopropyloxy-4- | 4 |
| | difluromethoxy)-phenyl]-2-[5-(2-(1-hydroxy- | |
| | 1-trifluoromethyl-2,2,2-trifluoro)ethyl)- | |
| | thiazolyl]ethyl}pyridine N-oxide | |
| L-869299 | (−)-1\|(R)-(−)-3-{2-[(3-cyclopropyloxy-4- | 4 |
| | difluromethoxy)phenyl]-2-[5-(2-(1-hydroxy-1- | |
| | trifluoromethyl-2,2,2- | |
| | trifluoro)ethyl)thiazolyl]ethyl}pyridine N- | |
| | Oxide | |
| Laprafylline | 8-[2-[4-(dicyclohexylmethyl)piperazin-1- | 4 |
| | yl]ethyl]-1-methyl-3-(2-methylpropyl)-7H- | |
| | purine-2,6-dione | |
| LAS 34179 | | 5 |
| LAS 37779 | | 4 |
| Levosimendan | U.S. Pat. No. 5,569,657 | 3 |
| Lirimilast | methanesulfonic acid 2-(2,4- | 4 |
| | dichlorophenylcarbonyl)-3-ureidobenzo-furan- | |
| | 6-yl ester | |
| Lixazinone | N-cyclohexyl-N-methyl-4-((1,2,3,5- | 3, 4 |
| | tetrahydro-2-oxoimidazo(2,1-b)quinazolin-7- | |
| | yl)oxy)-butanamide | |
| LPDE4 inhibitor | Bayer | 4 |
| Macquarimicin A | J Antibiot (Tokyo). 1995 Jun; 48(6): 462-6 | |
| MEM 1414 | | 4 |
| MERCK1 | (5R)-6-(4-{[2-(3-iodobenzyl)-3-oxocyclohex- | 3 |
| | 1-en-1-yl]amino}phenyl)-5-methyl-4,5- | |
| | dihydropyridazin-3(2H)-one; | |
| | dihydropyridazinone | |
| Mesopram | (5R)-5-(4-methoxy-3-propoxyphenyl)-5- | 4 |
| | methyl-2-oxazolidinone | |
| Milrinone | 6-dihydro-2-methyl-6-oxo-3,4′-bipyridine)-5- | 3, 4 |
| | carbonitrile (U.S. Pat. No. 4,478,836) | |
| MIMX | 1 8-methoxymethyl-3-isobutyl-1- | 1 |
| | methylxantine | |
| MN 001 | 4-[6-acetyl-3-[3-(4-acetyl-3-hydroxy-2- | 4 |
| | propylphenylthio)propoxy]-2- | |
| | propylphenoxy]butyric acid | |
| Mopidamol | U.S. Pat. No. 3,322,755 | 4 |
| MS 857 | 4-acetyl-1-methyl-7-(4-pyridyl)-5,6,7,8- | 3 |
| | tetrahydro-3(2H)-isoquinolinone | |
| Nanterinone | 6-(2,4-dimethyl-1H-imidazol-1-yl)-8-methyl- | 3 |
| | 2(1H)-quinolinone | |
| NCS 613 | J Pharmacol Exp Ther Boichot et al. 292 (2): | 4 |
| | 647 | |
| ND 1251 | | 4 |
| ND7001 | Neuro3D Pharmaceuticals | 2 |
| Nestifylline | 7-(1,3-dithiolan-2-ylmethyl)-1,3- | |
| | dimethylpurine-2,6-dione | |
| NIK 616 | | 4 |
| NIP 520 | | 3 |
| NM 702 | | 5 |
| NSP 306 | | 3 |
| NSP 513 | | 3 |
| NSP 804 | 4,5-dihydro-6-[4-[(2-methyl-3-oxo-1- | 3 |
| | cyclopentenyl)-amino]phenyl]-3(2H)- | |
| | pyridazinone | |
| NSP 805 | 4,5-dihydro-5-methyl-6-[4-[(2-methyl-3-oxo- | 3 |
| | 1-cyclopentenyl)amino]phenyl]-3(2H)- | |
| | pyridazinone | |
| NVP ABE 171 | | 4 |
| Oglemilast | N-(3,5-dichloropyridin-4-yl)-4- | 4 |
| | difluoromethoxy-8- | |
| | ((methylsulfonyl)amino)dibenzo(b,d)furan-1- | |
| | carboxamide | |
| Olprinone | 5-imidazo[2,1-f]pyridin-6-yl-6-methyl-2-oxo- | 3, 4 |
| | 1H-pyridine-3-carbonitrile | |
| ONO 1505 | 4-[2-(2-hydroxyethoxy)ethylamino]-2-(1H- | 5 |
| | imidazol-1-yl)-6-methoxy-quinazoline | |
| | methanesulphonate | |
| ONO 6126 | | 4 |
| OPC 33509 | (−)-6-[3-[3-cyclopropyl-3-[(1R,2R)-2- | 3 |
| | hydroxyclohexyl]ureido]-propoxy]-2(1H)- | |
| | quinolinone | |
| OPC 33540 | 6-[3-[3-cyclooctyl-3-[(1R[*],2R[*])-2- | 3 |
| | hydroxycyclohexyl]ureido]-propoxy]-2(1H)- | |
| | quinolinone | |
| ORG 20241 | N-hydroxy-4-(3,4-dimethoxyphenyl)-thiazole- | 3, 4 |
| | 2-carboximidamide | |
| ORG 30029 | N-hydroxy-5,6-dimethoxy- | 3, 4 |
| | benzo[b]thiophene-2-carboximide | |
| | hydrochloride | |
| ORG 9731 | 4-fluoro-N-hydroxy-5,6-dimethoxy- | 3, 4 |
| | benzo[b]thiophene-2-carboximidamide | |
| | methanesulphonate | |
| ORG 9935 | 4,5-dihydro-6-(5,6-dimethoxy-benzo[b]-thien- | 3 |
| | 2-yl)-methyl-1-(2H)-pyridazinone | |
| OSI 461 | N-benzyl-2-[(3Z)-6-fluoro-2-methyl-3- | 5 |
| | (pyridin-4-ylmethylidene)inden-1- | |
| | yl]acetamide hydrochloride | |
| Osthole | 7-methoxy-8-(3-methyl-2-butenyl)-2H-1- | 5 |
| | benzopyran-2-one | |
| Ouazinone | (R)-6-chloro-1,5-dihydro-3-methyl- | 3 |
| | imidazo[2,1-b]quinazolin-2-one | |
| PAB 13 | 6-bromo-8-(methylamino)imidazo[1,2- | |
| | a]pyrazine | |
| PAB 15 | 6-bromo-8-(ethylamino)imidazo[1,2- | |
| | a]pyrazine | |
| PAB 23 | 3-bromo-8-(methylamino)imidazo[1,2- | |
| | a]pyrazine | |
| Papaverine | 1-[(3.4-dimethoxyphenyl)-methyl]-6,7- | 5, 6, 7, 10 |
| | dimethoxyisoquinolone | |
| PDB 093 | | 4 |
| Pentoxifylline | 3,7-dimethyl-1-(5-oxohexyl)-3,7- | |
| | dihydropurine-2,6-dione (U.S. Pat. No. | |
| | 3,422,107) | |
| Piclamilast | 3-cyclopentyloxy-N-(3,5-dichloropyridin-4- | 4, 7 |
| | yl)-4-methoxy-benzamide | |
| Pimobendan | U.S. Pat. No. 4,361,563 | 3, 4 |
| Piroximone | 4-ethyl-1,3-dihydro-5-(4-pyridinylcarbonyl)- | 3 |
| | 2H-imidazol-2-one | |
| Prinoxodan | 6-(3,4-dihydro-3-methyl-2-oxoquinazolinyl)- | |
| | 4,5-dihydro-3-pyridazinone | |
| Propentofylline | U.S. Pat. No. 4,289,776 | 5 |
| Pumafentrine | rel-(M)-4-((4aR,10bS)-9-ethoxy- | 4 |
| | 1,2,3,4,4a,10b-hexahydro-8-methoxy-2- | |
| | methylbenzo(c)(1,6)naphthyridin-6-yl)-N,N- | |
| | bis(1-methylethyl)-benzamide | |
| R 79595 | N-cyclohexyl-N-methyl-2-[[[phenyl (1,2,3,5- | 3 |
| | tetrahydro-2 oxoimidazo [2,1-b]-quinazolin-7- | |
| | yl)methylene]amin]oxy]acetamide | |
| Revizinone | (E)-N-cyclohexyl-N-methyl-2- | 3 |
| | (((phenyl(1,2,3,5-tetrahydro-2- | |
| | oxoimidazo(2,1-b)quinazolin-7- | |
| | yl)methylene)amino)oxy)-acetamide | |
| Ro20-1724 | 4-(3-butoxy-4-methoxybenzyl)-2- | 4 |
| | imidazolidinone | |
| Roflumilast | 3-(cyclopropylmethoxy)-N-(3,5-dichloro-4- | 4, 5 |
| | pyridinyl)-4-(difluoromethoxy)-benzamide | |
| Rolipram | 4-(3-cyclopentyloxy-4-methoxyphenyl)-2- | 4 |
| | pyrrolidone (U.S. Pat. No. 4,193,926) | |
| RPL554 | 9,10-dimethoxy-2(2,4,6- | 3, 4 |
| | trimethylphenylimino)-3-(N-carbamoyl-2- | |
| | aminoethyl)-3,4,6,7-tetrahydro-2H- | |
| | pyrimido[6,1-a]isoquinolin-4-one | |
| RPL565 | 6,7-dihydro-2-(2,6-diisopropylphenoxy)-9,10- | 3, 4 |
| | dimethoxy-4H-pyrimido[6,1-a]isoquinolin-4- | |
| | one | |
| RPR 132294 | | 4 |
| RPR 132703 | | 4 |
| Saterinone | 1,2-dihydro-5-(4-(2-hydroxy-3-(4-(2- | 3 |
| | methoxyphenyl)-1- | |
| | piperazinyl)propoxy)phenyl)-6-methyl-2-oxo- | |
| | 3-pyridinecarbonitrile | |
| Satigrel | 4-cyano-5,5-bis(4-methoxyphenyl)-4- | 2, 3, 5 |
| | pentenoic acid (U.S. Pat. No. 4,978,767) | |
| SCA 40 | 6-bromo-8-methylaminoimidazol[1,2- | 3 |
| | a]pyrazine-2carbonitrile | |
| SCH 351591 | N-(3,5-dichloro-1-oxido-4-pyridinyl)-8- | 4 |
| | methoxy-2-(trifluoromethyl)-5-quinoline | |
| | carboxamide | |
| SCH 45752 | J Antibiot (Tokyo). 1993 Feb; 46(2): 207-13 | |
| SCH 46642 | | 5 |
| SCH 51866 | cis-5,6a,7,8,9,9a-hexahydro-2-(4- | 1, 5 |
| | (trifluoromethyl)phenylmethyl)-5-methyl- | |
| | cyclopent (4,5)imidazo(2,1-b)purin-4(3H)-one | |
| SCH 59498 | cis-2-hexyl-5-methyl-3,4,5,6a,7,8,9,9a- | 5 |
| | octahydrocyclopent[4,5]imidazo-[2,-1- | |
| | b]purin-4-one | |
| SDZ ISQ 844 | 6,7-dimethoxy-1-(3,4-dimethoxyphenyl)-3- | 3, 4 |
| | hydroxymethyl-3,4-dihydroisoquinoline | |
| SDZ MKS 492 | R(+)-(8-[(1-(3,4-dimethoxyphenyl)-2- | 3 |
| | hydroxyethyl)amino]-3,7-dihydro-7-(2- | |
| | methoxyethyl)-1,3-dimethyl-1H-purine-2,6- | |
| | dione | |
| Senazodan | | 3 |
| Siguazodan | N-cyano-N′-methyl-N″-[4-(1,4,5,6- | 3, 4 |
| | tetrahydro-4-methyl-6-oxo-3- | |
| | pyridazinyl)phenyl]guanidine | |
| Sildenafil | 5-[2-ethoxy-5-(4-methyl-1- | 5 |
| | piperazinylsulfonyl)phenyl]-1-methyl-3-n- | |
| | propyl-1,6-dihydro-7H-pyrazolo[4,3- | |
| | d]pyrimidin-7-one (U.S. Pat. No. 5,250,534) | |
| SK 3530 | | 5 |
| SKF 94120 | 5-(4-acetamidophenyl)pyrazin-2(1H)-one | 3 |
| SKF 95654 | ±-5-methyl-6-[4-(4-oxo-1,4-dihydropyridin-1- | 3 |
| | yl)phenyl]-4,5-dihydro-3(2H)-pyridazinone | |
| SKF 96231 | 2-(2-propoxyphenyl)-6-purinone | 3, 4, 5 |
| SLX 2101 | | 5 |
| Sulmazole | U.S. Pat. No. 3,985,891 | 3 |
| T 0156 | 2-(2-methylpyridin-4-yl)methyl-4-(3,4,5- | 5 |
| | trimethoxyphenyl)-8-(pyrimidin-2- | |
| | yl)methoxy-1,2-dihydro-1-oxo-2,7- | |
| | naphthyridine-3-carboxylic acid methyl ester | |
| | hydrochloride | |
| T 1032 | methyl-2-(4-aminophenyl)-1,2-dihydro-1-oxo- | 5 |
| | 7-(2-pyridylmethoxy)-4-(3,4,5- | |
| | trimethoxyphenyl)-3-isoquinoline carboxylate | |
| | sulfate | |
| T 440 | 6,7-diethoxy-1-[1-(2-methoxyethyl)-2-oxo- | 4 |
| | 1,2-dihydropyridin-4-yl]naphthalene-2,3- | |
| | dimethanol | |
| Tadalafil | (6R,12aR)-6-(1,3-benzodioxol-5-yl)-2-methyl- | 4, 5 |
| | 2,3,6,7,12,12a- | |
| | hexahydropyrazino[1,2,1,6]pyrido[3,4- | |
| | b]indole-1,4-dione | |
| Tetomilast | 6-(2-(3,4-diethoxyphenyl)-4-thiazolyl)-2- | 4 |
| | pyridinecarboxylic acid | |
| Theophylline | 3,7-dihydro-1,3-dimethyl-1H-purine-2,6-dione | Not selective |
| Tibenelast | 5,6-diethoxybenzo(B)thiophene-2-carboxylic | 4 |
| | acid | |
| Toborinone | (+/−)-6-[3-(3,4-dimethoxybenzylamino)-2- | 3 |
| | hydroxypropoxy]-2(1H)-quinolinone | |
| Tofimilast | 9-cyclopentyl-7-ethyl-6,9-dihydro-3-(2- | 4 |
| | thienyl)-5H-pyrazolo(3,4-c)-1,2,4-triazolo(4,3- | |
| | a)pyridine | |
| Tolafentrine | N-[4-[(4aS, 10bR)-8,9-dimethoxy-2-methyl- | 3, 4 |
| | 3,4,4a,10b-tetrahydro-1H-pyrido[4,3- | |
| | c]isoquinolin-6-yl]phenyl]-4- | |
| | methylbenzenesulfonamide | |
| Torbafylline | 7-(ethoxymethyl)-3,7-dihydro-1-(5-hydroxy-5- | 4 |
| | methylhexyl)-3-methyl-1-H-purine-2,6-dione | |
| Trequinsin | 2,3,6,7-tetrahydro-9,10-dimethoxy-3-methyl- | 2, 3, 4 |
| | 2-((2,4,6-trimethylphenyl)imino)-4H- | |
| | pyrimido(6,1-a)isoquinolin-4-one | |
| UCB 29936 | | 4 |
| UDCG 212 | 5-methyl-6-[2-(4-oxo-1-cyclohexa-2,5- | 3 |
| | dienylidene)-1,3-dihydrobenzimidazol-5-yl]- | |
| | 4,5-dihydro-2H-pyridazin-3-one | |
| Udenafil | 3-(1-methyl-7-oxo-3-propyl-4H-pyrazolo[5,4- | 5 |
| | e]pyrimidin-5-yl)-N-[2-(1-methylpyrrolidin-2- | |
| | yl)ethyl]-4-propoxybenzenesulfonamide | |
| UK 114542 | 5-[2-ethoxy-5-(morpholinylacetyl) phenyl]- | 5 |
| | 1,6-dihydro-1-methyl-3-propyl-7H-pyrazolo | |
| | [4,3-d]-pyrimidin-7-one | |
| UK 343664 | 3-ethyl-5-(5-((4-ethylpiperazino)sulphonyl)-2- | 5 |
| | propoxyphenyl)-2-(2-pyridylmethyl)-6,7- | |
| | dihydro-2H-pyrazolo(4,3-d)pyrimidin-7-one | |
| Zaprinast | 2-o-propoxyphenyl-8-azapurine-6-one | 1, 5 |
| Zardaverine | 6-(4-(difluoromethoxy)-3-methoxyphenyl)- | 3, 4 |
| | 3(2H)-Pyridazinone | |
| Zindotrine | 8-methyl-6-(1-piperidinyl)-1,2,4-triazolo(4,3- | |
| | b)pyridazine | |

Additional PDE inhibitors are shown in Table 6.

TABLE 6

| | | |
|---|---|---|
| 5E3623 | A 021311 | A 906119 |
| BFGP 385 | BY 244 | CC 11050 |
| CP 166907 | CP 77059 | CT 1579 |
| CT 1786 | HFV 1017 | IPL 423088 |
| IWF 12214 | K 123 | KF 31334 |
| MKS 213492 | MX 2120 | N 3601 |
| ORG 20494 | REN 1053 | RP 116474 |
| Trombodipine | UK 66838 | Vasotrope |

Other PDE 1 inhibitors are described in U.S. Patent Application Nos. 20040259792 and 20050075795, incorporated herein by reference. Other PDE 2 inhibitors are described in U.S. Patent Application No. 20030176316, incorporated herein by reference. Other PDE 3 inhibitors are described in the following patents and patent applications: EP 0 653 426, EP 0 294 647, EP 0 357 788, EP 0 220 044, EP 0 326 307, EP 0 207 500, EP 0 406 958, EP 0 150 937, EP 0 075 463, EP 0 272 914, and EP 0 112 987, U.S. Pat. Nos. 4,963,561; 5,141,931, 6,897,229, and 6,156,753; U.S. Patent Application Nos. 20030158133, 20040097593, 20060030611, and 20060025463; WO 96/15117; DE 2825048; DE 2727481; DE 2847621; DE 3044568; DE 2837161; and DE 3021792, each of which is incorporated herein by reference. Other PDE 4 inhibitors are described in the following patents, patent applications, and references: U.S. Pat. Nos. 3,892,777, 4,193,926, 4,655,074, 4,965,271, 5,096,906, 5,124,455, 5,272,153, 6,569,890, 6,953,853, 6,933,296, 6,919,353, 6,953,810, 6,949,573, 6,909,002, and 6,740,655; U.S. Patent Application Nos. 20030187052, 20030187257, 20030144300, 20030130254, 20030186974, 20030220352, 20030134876, 20040048903, 20040023945, 20040044036, 20040106641,

| | | PDE |
|---|---|---|
| Compound | Synonym | Activity |
| UK 357903 | 1-ethyl-4-{3-[3-ethyl-6,7-dihydro-7-oxo-2-(2- | 5 |
| | pyridylmethyl)-2H-pyrazolo[4,3-d]pyrimidin- | |
| | 5-yl]-2-(2-methoxyethoxy)5- | |
| | pyridylsulphonyl} piperazine | |
| UK 369003 | | 5 |
| V 11294A | 3-((3-(cyclopentyloxy)-4- | 4 |
| | methoxyphenyl)methyl)-N-ethyl-8-(1- | |
| | methylethyl)-3H-purin-6-amine | |
| | monohydrochloride | |
| Vardenafil | 2-(2-ethoxy-5-(4-ethylpiperazin-1-yl-1- | 5 |
| | sulfonyl)phenyl)-5-methyl-7-propyl-3H- | |
| | imidazo(5,1-f)(1,2,4)triazin-4-one | |
| Vesnarinone | U.S. Pat. No. 4,415,572 | 3, 5 |
| Vinpocetine | (3-alpha,16-alpha)-eburnamenine-14- | 1, 3, 4 |
| | carboxylic acid ethyl ester | |
| WAY 122331 | 1-aza-10-(3-cyclopentyloxy-4- | 4 |
| | methoxyphenyl)-7,8-dimethyl-3- | |
| | oxaspiro[4.5]dec-7-en-2-one | |
| WAY 127093B | [(3S)-3-(3-cyclopentyloxy-4-methoxyphenyl)- | 4 |
| | 2-methyl-5-oxopyrazolidinyl]-N-(3- | |
| | pyridylmethyl)carboxamide | |
| WIN 58237 | 1-cyclopentyl-3-methyl-6-(4- | 5 |
| | pyridinyl)pyrazolo (3,4-d)pyrimidin-4(5H)- | |
| | one | |
| WIN 58993 | 5-methyl-6-pyridin-4-yl-3H-[1,3]thiazolo[5,4- | 3 |
| | e]pyridin-2-one | |
| WIN 62005 | 5-methyl-6-pyridin-4-yl-1,3- | 3 |
| | dihydroimidazo[4,5-e]pyridin-2-one | |
| WIN 62582 | 6-pyridin-4-yl-5-(trifluoromethyl)-1,3- | 3 |
| | dihydroimidazo[4,5-b]pyridin-2-one | |
| WIN 63291 | 6-methyl-2-oxo-5-quinolin-6-yl-1H-pyridine- | 3 |
| | 3-carbonitrile | |
| WIN 65579 | 1-cyclopentyl-6-(3-ethoxy-4-pyridinyl)-3- | 5 |
| | ethyl-1,7-dihydro-4H-pyrazolo[3,-4- | |
| | d]pyrimidin-4-one | |
| Y 20487 | 6-(3,6-dihydro-2-oxo-2H-1,3,4-thiadiazin-5- | 3 |
| | yl)-3,4-dihydro-2(1H)-quinolinone | |
| YM 58997 | 4-(3-bromophenyl)-1,7-diethylpyrido[2,3- | 4 |
| | d]pyrimidin-2(1H)-one | |
| YM 976 | 4-(3-chlorophenyl)-1,7-diethylpyrido(2,3- | 4 |
| | d)pyrimidin-2(1H)-one | |
| Z 15370A | | 4 |
| Zaprinast | 1,4-dihydro-5-(2-propoxyphenyl)-7H-1,2,3- | 5 |
| | triazolo[4,5-d]pyrimidine-7-one | |

20040097593, 20040242643, 20040192701, 20040224971, 20040220183, 20040180900, 20040171798, 20040167199, 20040146561, 20040152754, 20040229918, 20050192336, 20050267196, 20050049258, 20060014782, 20060004003, 20060019932, 20050267196, 20050222207, 20050222207, 20060009481; International Publication No. WO 92/079778; and Molnar-Kimber, K. L. et al. J. Immunol., 150:295 A (1993), each of which is incorporated herein by reference. Other PDE 5 inhibitors that can be used in the methods, compositions, and kits of the invention include those described in U.S. Pat. Nos. 6,992,192, 6,984,641, 6,960,587, 6,943,166, 6,878,711, and 6,869,950, and U.S. Patent Application Nos. 20030144296, 20030171384, 20040029891, 20040038996, 20040186046, 20040259792, 20040087561, 20050054660, 20050042177, 20050245544, 20060009481, each of which is incorporated herein by reference. Other PDE 6 inhibitors that can be used in the methods, compositions, and kits of the invention include those described in U.S. Patent Application Nos. 20040259792, 20040248957, 20040242673, and 20040259880, each of which is incorporated herein by reference. Other PDE 7 inhibitors that can be used in the methods, compositions, and kits of the invention include those described in the following patents, patent application, and references: U.S. Pat. Nos. 6,838,559, 6,753,340, 6,617,357, and 6,852,720; U.S. Patent Application Nos. 20030186988, 20030162802, 20030191167, 20040214843, and 20060009481; International Publication WO 00/68230; and Martinez et al., J. Med. Chem. 43:683-689 (2000), each of which is incorporated herein by reference. Other PDE inhibitors that can be used in the methods, compositions, and kits of the invention are described in U.S. Pat. No. 6,953,774.

In certain embodiments, more than one PDE inhibitor may be employed in the invention so that the combination has activity against at least two of PDE 2, 3, 4, and 7. In other embodiments, a single PDE inhibitor having activity against at least two of PDE 2, 3, 4, and 7 is employed.

### Additional Compounds

A BAR agonist may also be employed with an antiproliferative compound for the treatment of a B-cell proliferative disorder. Additional compounds that are useful in such methods include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin A receptor antagonist, retinoic acid receptor agonists, immuno-modulators, hormonal and antihormonal agents, photodynamic agents, tyrosine kinase inhibitors, antisense compounds, corticosteroids, HSP90 inhibitors, proteosome inhibitors (for example, NPI-0052), CD40 inhibitors, anti-CSI antibodies, FGFR3 inhibitors, VEGF inhibitors, MEK inhibitors, cyclin D inhibitors (e.g., cyclin D1), NF-kB inhibitors and pathway modulators, anthracyclines, histone deacetylase inhibitors, kinesin inhibitors, phosphatase inhibitors, COX2 inhibitors, mTOR inhibitors, AKT inhibitors, PI3K inhibitors, TRAF inhibitors, statins, mitotic kinase inhibitors, KSP inhibitors, cyclin dependent kinase inhibitors (e.g., flavopiridol), inhibitors of anti-apoptotic proteins, e.g., BCL-2 (e.g., oblimereson), immune therapies (e.g., anti-CD20 or anti-CD22), calcineurin antagonists, IMiDs, or other agents used to treat proliferative diseases. Specific examples are shown in Tables 7 and 8.

TABLE 7

| | | |
|---|---|---|
| 17-AAG (KOS-953) | 1D09C3 | Activated T cells |
| AE 941 | Aflibercept | AG 490 |
| Alemtuzumab | Alitretinoin oral - Ligand | Alvocidib |
| | Pharmaceuticals | |
| AMG162 (denosumab, | Anti-CD38 antibodies | Anti-CD38 monoclonal |
| osteoprotegerin, OPG) | | antibody AT13/5 |
| Anti-CD46 human | Anti-CD5 monoclonal | Anti-HM1-24 monoclonal |
| monoclonal antibodies | antibodies | antibody |
| Anti-MUC1 monoclonal | Antineoplaston A10 - | Antineoplaston AS2 1 - |
| antibody - United | injection | injection |
| Therapeutics/ViRexx Medical | | |
| Corp | | |
| AP23573 | APC 8020 | Aplidin ® |
| Apo2L/TRAIL | Apomine ™ (SR-45023A) | AR20.5 |
| Arsenic trioxide | AT 101 | Atacicept (TACI-Ig) |
| Atiprimod | Atiprimod | ATN 224 |
| Avastin ™ (bevacizumab, | AVN944 | Azathioprine |
| rhuMAb-VEGF) | | |
| B-B4-DMI | BCX-1777 (forodesine) | Belinostat |
| Bendamustine (SDX-105) | Benzylguanine | Beta alethine |
| Bexxar (Iodine I 131 | BIBF-1120 | Bortezomib (VELCADE ®) |
| tositumomab) | | |
| Breva-Rex ® | Brostallicin | Bufexamac |
| BX 471 | Cadi-05 | Cancer immunotherapies - |
| | | Cell Genesys |
| Carmustine | CC 4047 | CC007 |
| CC11006 | CCI-779 | CD74-targeted therapeutics |
| Celebrex (celecoxib) | CERA (Continuous | CHIR-12.12 |
| | Erythropoiesis Receptor | |
| | Activator) | |
| cKap | Clodronic acid | CNTO 328 |
| CP 751871 | CRB 15 | Curcumin |
| Cyclophosphamide | Danton | Darinaparsin |
| Dasatinib | Daunorubicin liposomal | Defibrotide |
| Dexamethasone | Dexniguldipine | DHMEQ |
| Dimethylcelecoxib | DOM1112 | Doxorubicin |
| Doxorubicin liposomal (PNU- | Doxycycline | Elsilimomab |
| 108112) - ALZA | | |
| EM164 | ENMD 0995 | Erbitux, cetuximab |
| Ethyol ® (amifostine) | Etoposide | Fibroblast growth factor |
| | | receptor inhibitors |
| Fludarabine | Fluphenazine | FR901228 (depsipeptide) |
| G3139 | Gallium Maltolate | GCS 100 |
| GCS-100 | GCS-100LE | GRN 163L |
| GVAX ® Myeloma Vaccine | GW654652 | GX15-070 |
| HGS-ETR1 (TRM-1, | Highly purified hematopoietic | Histamine dihydrochloride |
| mapatumumab) | stem cells | injection - EpiCept |
| | | Corporation |
| hLL1 | Holmium-166 DOTMP | HSV thymidine kinase gene |
| | | therapy |
| HuLuc63 | HuMax-CD38 | huN901-DM1 |
| Idarubicin | Imexon - Heidelberg Pharma | Imexon (plimexon) - |
| | | AmpliMed |
| IMMU 110 | Incadronic acid | Interferon-alpha-2b |
| IPI 504 | Irinotecan | ISIS 345794 |
| Isotretinoin | ITF 2357 | Kineret ™ (anakinra) |
| KOS-1022 (alvespimycin | KRX-0401, perifosine | LAF 389 |
| HCl; 17-DMAG; NSC707545) | | |
| LBH589 | Lenalidomide (Revlimid ®) | Lestaurtinib |
| LPAAT-β inhibitors | Lucatumumab | LY2181308 |
| Melphalan | Menogaril | Midostaurin |
| Minodronic acid | MK 0646 | MOR202 |
| MS-275 | Multiple myeloma vaccine - | MV-NIS |
| | GTC | |
| Myeloma vaccine - Onyvax | MyelomaCide | Mylovenge |
| Nexavar ® (BAY 43-9006, | Noscapine | NPI 0052 |
| sorafenib, sorafenib tosylate) | | |
| O-6-benzyl-guanine | Obatoclax | Oblimersen |
| OGX-427 | Paclitaxel | Pamidronic acid |
| Panzem ™(2-meth- | Parthenolide | PD173074 |
| oxyestradiol, 2ME2) | | |
| Phosphostim | PI 88 | Plitidepsin |
| PR-171 | Prednisone | Proleukin ® (IL-2, Interleukin- |
| | | 2) |
| PX-12 | PXD101 | Pyroxamide |
| Quadramet ® (EDTMP, | RAD001 (everolimus) | Radiolabelled BLyS |
| samarium-153 ethylene | | |
| diamine tetramethylene | | |
| phosphonate Samarium) | | |
| RANK-Fc | Rituximab | Romidepsin |
| RTA402 | Samarium 153 SM lexidronam | Sant 7 |
| SCIO-469 | Scriptaid | SD-208 |
| SDX-101 | Seleciclib | SF1126 |
| SGN 40 | SGN-70 | Sirolimus |
| Sodium Stibogluconate | Spironolactone | SR 31747 |
| (VQD-001) | | |
| SU5416 | SU6668 | Tanespimycin |
| Temodar ® (temozolomide) | Thalidomide | Thrombospondin-1 |
| Tiazofurine | Tipifarnib | TKI 258 |
| Tocilizumab (atlizumab) | Topotecan | Tretinoin |
| Valspodar | Vandetanib (Zactima ™) | Vatalanib |
| VEGF Trap (NSC 724770) | Vincristine | Vinorelbine |
| VNP 4010M | Vorinostat | Xcytrin (motexafin |
| | | gadolinium) |
| XL999 | ZIO-101 | Zoledronic acid |
| ZRx 101 | Trichostatin A | Suberoylanilide hydroxamic |
| | | acid (SAHA) |

BAR agonists may also be employed with combinations of antiproliferative compounds. Such additional combinations include CHOP (cyclophosphamide, vincristine, doxorubicin, and prednisone), VAD (vincristine, doxorubicin, and dexamethasone), MP (melphalan and prednisone), DT (dexamethasone and thalidomide), DM (dexamethasone and melphalan), DR (dexamethasone and Revlimid (lenalidomide)), DV (dexamethasone and Velcade), RV (Revlimid and Velcade), and cyclophosphamide and etoposide.

Additional compounds related to bortezomib that may be used in the invention are described in U.S. Pat. Nos. 5,780,454, 6,083,903, 6,297,217, 6,617,317, 6,713,446, 6,958,319, and 7,119,080. Other analogs and formulations of bortezomib are described in U.S. Pat. Nos. 6,221,888, 6,462,019, 6,472,158, 6,492,333, 6,649,593, 6,656,904, 6,699,835, 6,740,674, 6,747,150, 6,831,057, 6,838,252, 6,838,436, 6,884,769, 6,902,721, 6,919,382, 6,919,382, 6,933,290, 6,958,220, 7,026,296, 7,109,323, 7,112,572, 7,112,588, 7,175,994, 7,223,554, 7,223,745, 7,259,138, 7,265,118, 7,276,371, 7,282,484, and 7,371,729.

Additional compounds related to lenalidomide that may be used in the invention are described in U.S. Pat. Nos. 5,635,517, 6,045,501, 6,281,230, 6,315,720, 6,555,554, 6,561,976, 6,561,977, 6,755,784, 6,908,432, 7,119,106, and 7,189,740. Other analogs and formulations of lenalidomide are described in U.S. Patent Nos. RE40,360, 5,712,291, 5,874,448, 6,235,756, 6,281,230, 6,315,720, 6,316,471, 6,335,349, 6,380,239, 6,395,754, 6,458,810, 6,476,052, 6,555,554, 6,561,976, 6,561,977, 6,588,548, 6,755,784, 6,767,326, 6,869,399, 6,871,783, 6,908,432, 6,977,268, 7,041,680, 7,081,464, 7,091,353, 7,115,277, 7,117,158, 7,119,106, 7,141,018, 7,153,867, 7,182,953, 7,189,740, 7,320,991, 7,323,479, and 7,329,761.

Further antiproliferative compounds that may be employed with the combinations of the invention are shown in Table 8.

TABLE 8

| | | |
|---|---|---|
| 6-Mercaptopurine | Gallium (III) Nitrate Hydrate | Altretamine |
| Anastrozole | Bicalutamide | Bleomycin |
| Busulfan | Camptothecin | Capecitabine |
| Carboplatin | Chlorambucil | Cisplatin |
| Cladribine | Cytarabine | Dacarbazine |
| Dactinomycin | Docetaxel | Epirubicin, e.g., |
| | | Hydrochloride |
| Estramustine | Exemestane | Floxuridine |
| Fluorouracil | Flutamide | Fulvestrant |
| Gemcitabine, e.g., | Hydroxyurea | Ifosfamide |
| Hydrochloride | | |
| Imatinib | Iressa | Ketoconazole |
| Letrozole | Leuprolide | Levamisole |
| Lomustine | Mechlorethamine, e.g., | Megestrol, e.g., |
| | Hydrochloride | Acetate |
| Methotrexate | Mitomycin | Mitoxantrone, e.g., |
| | | Hydrochloride |
| Nilutamide | Oxaliplatin | Pemetrexed |
| Plicamycin | Prednisolone | Procarbazine |
| Raltitrexed | Rofecoxib | Streptozocin |
| Suramin | Tamoxifen Citrate | Teniposide |
| Testolactone | Thioguanine | Thiotepa |
| Toremifene | Vinblastine, e.g., Sulfate | Vindesine |

Preferred antiproliferative compounds include vincristine, lenalidomide, bortezomib, prednisolone, doxorubicin, cyclophosphamide, dexamethasone, melphalan, cyclophosphamide, etoposide, cytarabine, cisplatin, fludarabine, rituxan, thalidomide, methlyprednisolone, doxil (pegylated doxorubicin), panobinostat, tanespimycin, oblimersen, valspodar, and vorinostat. Other preferred compounds include HDAC inhibitors and HSP90 inhibitors.

Additional preferred antiproliferative compounds include pentostatin, chlorambucil, alemtuzumab, mitoxantrone, carmustine, gemcitabine, procarbazine, ifosfamide, mesma, oxaliplatin, and cladribine.

A BAR agonist may also be employed with IL-6 for the treatment of a B-cell proliferative disorder. If not by direct administration of IL-6, patients may be treated with agent(s) to increase the expression or activity of IL-6. Such agents may include other cytokines (e.g., IL-1 or TNF), soluble IL-6 receptor a (sIL-6R α), platelet-derived growth factor, prostaglandin E1, forskolin, cholera toxin, dibutyryl cAMP, or IL-6 receptor agonists, e.g., the agonist antibody MT-18, K-7/D-6, and compounds disclosed in U.S. Pat. Nos. 5,914,106, 5,506,107, and 5,891,998.

### Combinations

The invention includes the individual combination of each BAR agonist with each A2A receptor agonist, each PDE inhibitor, and each antiproliferative compound provided herein, as if each combination were explicitly stated. In a particular example, the BAR agonist is arbutaline, arfomoterol, bambuterol, bitolterol, broxaterol, clenbuterol, fenoterol, formoterol, hexoprenaline, indacaterol, isoetharine, isoproterenol, levalbuterol, meluadrine, metaproterenol, nylidrin, picumeterol, pirbuterol, procaterol, reproterol, rimiterol, ritodrine, salbutamol, salmeterol, tulobuterol, terbutaline, or xamoterol, and the A2A agonist, PDE inhibitor, or antiproliferative compounds is any one or more of those described herein. For example, preferred A2A agonists include adenosine, regadenoson, apadenoson, sonedenoson, MRE-0094, BVT-115959, UK-432097, acadesine, tocladesine, CGS-21680C and CGS-21680, spongosine, binodenoson, HE-NECA, IB-MECA, CI-IB-MECA, NECA, ATL-313, ATL-1222, and DPMA. Preferred PDE inhibitors include trequinsin, zardaverine, roflumilast, rolipram, cilostazol, milrinone, papaverine, BAY 60-7550, and BRL-50481. Preferred antiproliferative compounds include vincristine, lenalidomide, bortezomib, prednisolone, doxorubicin, cyclophosphamide, dexamethasone, melphalan, cyclophosphamide, etoposide, cytarabine, cisplatin, fludarabine, rituxan, thalidomide, methlyprednisolone, doxil (pegylated doxorubicin), panobinostat, tanespimycin, oblimersen, valspodar, and vorinostat or pentostatin, chlorambucil, alemtuzumab, mitoxantrone, carmustine, gemcitabine, procarbazine, ifosfamide, mesma, oxaliplatin, and cladribine. Other preferred antiproliferative compounds include HDAC inhibitors and HSP90 inhibitors, as described herein.

Exemplary combinations are shown in Table 9.

TABLE 9

| BAR agonist | Additional Compound |
|---|---|
| salmeterol | dexamethasone |
| formoterol | dexamethasone |
| procaterol | dexamethasone |
| salbutamol | dexamethasone |
| levalbuterol | dexamethasone |
| salmeterol | melphalan |
| formoterol | melphalan |
| procaterol | melphalan |
| salbutamol | melphalan |
| levalbuterol | melphalan |
| salmeterol | lenalidomide |
| formoterol | lenalidomide |
| procaterol | lenalidomide |
| salbutamol | lenalidomide |
| levalbuterol | lenalidomide |
| salmeterol | doxorubicin |
| formoterol | doxorubicin |
| procaterol | doxorubicin |
| salbutamol | doxorubicin |
| levalbuterol | doxorubicin |
| salmeterol | bortezomib |
| formoterol | bortezomib |
| procaterol | bortezomib |
| salbutamol | bortezomib |
| levalbuterol | bortezomib |

In certain embodiments, a BAR agonist, e.g., bambuterol, bitolterol, clenbuterol, formoterol, isoproterenol, metaproterenol, pirbuterol, salbutamol, or salmeterol, terbutaline, is not administered with a stem cell mobilizer, e.g., AMD3100, cyclophosphamide, stem cell factor (SCF), filgrastim, or ancestim. In other embodiments, a BAR agonist, e.g., bambuterol, bitolterol, isoetharine, isoproterenol, metaproterenol, salbutamol, or terbutaline, is not administered with an mTOR inhibitor and capecitabine. In further embodiments, a BAR agonist, e.g., bambuterol, terbutaline, pirbuterol, bitolterol, formoterol, salmeterol, or salbutamol, is not administered with a PDE4 inhibitor.

The following combinations may be excluded from treatment of a B-cell proliferative disorder in certain embodiments: salmeterol, fluticasone, and CHOP or bortezomib for treatment of mantle cell lymphoma; salbutamol and VAD for multiple myeloma; salmeterol, beclomethasone, prednisone, and melphalan for multiple myeloma; salmeterol, beclomethasone, prednisone, clodronate, salbutamol, and melphalan for multiple myeloma; and salbutamol, beclomethasone, melphalan, prednisone, and pamidronate for multiple myeloma.

### B-Cell Proliferative Disorders

B-cell proliferative disorders include B-cell cancers and autoimmune lymphoproliferative disease. B-cell cancers that can be treated according to the methods of the invention include B-cell CLL, B-cell prolymphocyte leukemia, lymphoplasmacytic lymphoma, mantle cell lymphoma, follicular lymphoma, extranodal marginal zone B-cell lymphoma of mucosa-associated lymphoid tissue (MALT type), nodal marginal zone lymphoma, splenic marginal zone lymphoma, hairy cell leukemia, plasmacytoma, diffuse large B-cell lymphoma, Burkitt lymphoma, multiple myeloma, indolent myeloma, smoldering myeloma, monoclonal gammopathy of unknown significance (MGUS), B-cell non-Hodgkin's lymphoma, small lymphocytic lymphoma, monoclonal immunoglobin deposition diseases, heavy chain diseases, mediastinal (thymic) large B-cell lymphoma, intravascular large B-cell lymphoma, primary effusion lymphoma, lymphomatoid granulomatosis, precursor B-lymphoblastic leukemia/lymphoma, Hodgkin's lymphoma (e.g., nodular lymphocyte predominant Hodgkin's lymphoma, classical Hodgkin's lymphoma, nodular sclerosis Hodgkin's lymphoma, mixed cellularity Hodgkin's lymphoma, lymphocyte-rich classical Hodgkin's lymphoma, and lymphocyte depleted Hodgkin's lymphoma), post-transplant lymphoproliferative disorder, and Waldenstrom's macroglobulinemia. A preferred B-cell cancer is multiple myeloma. Other preferred B-cell cancers include mantle cell lymphoma, Burkitt lymphoma, diffuse large B-cell lymphoma, chronic lymphocytic leukemia, and follicular lymphoma. Other such disorders are known in the art.

In certain embodiments, when the B-cell proliferative disorder is B-CLL, the BAR agonist is not formoterol, isoproterenol (e.g., in combination with a PDE inhibitor), or salmeterol.

In other embodiments, when the B-cell proliferative disorder is doxorubicin resistant multiple myeloma, the BAR agonist is not salbutamol. In further embodiments, when the B-cell proliferative disorder is mantle cell lymphoma, the BAR agonist is not salmeterol.

### Administration

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment.

When combinations are employed, the compounds may be administered within 28 days of each other, within 14 days of each other, within 10 days of each other, within five days of each other, within twenty-four hours of each other, or simultaneously. The compounds may be formulated together as a single composition, or may be formulated and administered separately. Each compound may be administered in a low dosage or in a high dosage, each of which is defined herein.

Routes of administration for the various embodiments include, but are not limited to, topical, transdermal, and systemic administration (such as, intravenous, intramuscular, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration. In one example, RPL554 is administered intranasally.

In certain embodiments, administration of a BAR agonist occurs by a route other than topical, transdermal, or inhalation. Preferred routes for BAR agonists are oral and intravenous.

In combination therapy, the dosage and frequency of administration of each component of the combination can be controlled independently. For example, one compound may be administered three times per day, while a second compound may be administered once per day. Combination therapy may be given in on-and-off cycles that include rest periods so that the patient's body has a chance to recover from any as yet unforeseen side effects. The compounds may also be formulated together such that one administration delivers both compounds.

### Formulation of Pharmaceutical Compositions

The administration of a combination of the invention may be by any suitable means that results in suppression of proliferation at the target region. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 21st edition, 2005, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Preferred formulations for BAR agonists include those suitable for oral or intravenous administration.

Each compound of the combination may be formulated in a variety of ways that are known in the art. For example, all agents may be formulated together or separately. Desirably, all agents are formulated together for the simultaneous or near simultaneous administration of the agents. Such co-formulated compositions can include a BAR agonist and any additional compound, e.g., A2A receptor agonist, PDE inhibitor, or antiproliferative compound, formulated together in the same pill, capsule, liquid, etc. It is to be understood that, when referring to the formulation of combinations, the formulation technology employed is also useful for the formulation of the individual agents of the combination, as well as other combinations of the invention. By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like. Any kit of the invention may also include instructions on the administration of the included compounds for the treatment of a B-cell proliferative disorder.

### Dosages

Generally, the dosage of the BAR agonist is 0.1 μg to 50 mg per day, preferably 1 μg to 0.1 mg. The dosage of the A2A receptor agonist is 0.1 mg to 500 mg per day, e.g., about 50 mg per day, about 5 mg per day, or desirably about 1 mg per day. The dosage of the PDE inhibitor is, for example, 0.1 to 2000 mg, e.g., about 200 mg per day, about 20 mg per day, or desirably about 4 mg per day.

Dosages of antiproliferative compounds are known in the art and can be determined using standard medical techniques.

Administration of each drug in the combination can, independently, be one to four times daily for one day to one year.

The following examples are to illustrate the invention. They are not meant to limit the invention in any way.

### EXAMPLES

### Example 1

### Materials and Methods

### Tumor Cell Culture

MM.1S, MM.1R, EJM, RPMI-8226, INA-6, and ANBL-6 multiple myeloma cell lines were cultured at 37° C. and 5% CO₂ in RPMI-1640 media supplemented with 10% FBS. INA-6 and ANBL-6 culture media was supplemented with 10 ng/ml IL-6. The diffuse large B-cell lymphoma line OCI-ly10 was cultured at 37° C. and 5% CO₂ in Iscoves media supplemented with 20% human serum. MM.1R and OCI-ly10 cells were provided by the Dana Farber Cancer Institute. MM.1S cells were provided by Steven Rosen, Northwestern University. INA-6 and ANBL-6 cells were from Robert Orlowski, M.D. Anderson Cancer Center. RPMI-8226 and EJM cells were from DSMZ (Cat #'s ACC 402 and ACC 560). GA-10 cells were obtained from ATCC(CRL-2392). Human coronary artery endothelial cells (HCAEC) were obtained from Lonza and cultured as recommended by the supplier.

### Compounds

Compounds were prepared in DMSO at 1000× the highest desired concentration. Master plates were generated consisting of serially diluted compounds in 2- or 3-fold dilutions in 384-well format. For single agent dose response curves, the master plates consisted of 9 individual compounds at 12 concentrations in 2- or 3-fold dilutions. For combination matrices, master plates consisted of individual compounds at 6 or 9 concentrations at 2- or 3-fold dilutions.

### Anti-Proliferation Assay

Cells were added to 384-well plates 24 hours prior to compound addition, and each well contained 2000 cells in 35 μL of media. Master plates were diluted 100× (1 μL into 100 μL) into 384-well dilution plates containing only cell culture media. 4.5 μL from each dilution plate was added to each assay plate for a final dilution of 1000×. To obtain combination data, two master plates were diluted into the assay plates. Following compound addition, assay plates were kept at 37° C. and 5% CO₂ for 72 hours. 30 μL of ATPLite (Perkin Elmer) at room temperature was then added to each well. Final amount of ATP was quantified within 30 minutes using ATPLite luminescent read-out on an Envision 2103 Multilabel Reader (Perkin Elmer). Measurements were taken at the top of the well using a luminescence aperture and a read time of 0.1 seconds per well.

The percent inhibition (% I) for each well was calculated using the following formula:

%I=[(avg. untreated wells−treated well)/(avg. untreated wells)]×100.

The average untreated well value (avg. untreated wells) is the arithmetic mean of 40 wells from the same assay plate treated with vehicle alone. Negative inhibition values result from local variations in treated wells as compared to untreated wells.

Single agent activity was characterized by fitting a sigmoidal function of the form I=IₘₐₓC^{α}/[C^{α}+EC₅₀^{α}], with least squares minimization using a downhill simplex algorithm (C is the concentration, EC₅₀ is the agent concentration required to obtain 50% of the maximum effect, and α is the sigmoidicity). The uncertainty of each fitted parameter was estimated from the range over which the change in reduced chi-squared was less than one, or less than minimum reduced chi-squared if that minimum exceeded one, to allow for underestimated σ_{I} errors.

Single agent curve data were used to define a dilution series for each compound to be used for combination screening in a 6×6 or 9×9 matrix format. Using a dilution factor f of 2, 3, or 4, depending on the sigmoidicity of the single agent curve, five dose levels were chosen with the central concentration close to the fitted EC₅₀. For compounds with no detectable single agent activity, a dilution factor of 4 was used, starting from the highest achievable concentration.

The Loewe additivity model was used to quantify combination effects. Combinations were ranked initially by Additivity Excess Volume, which is defined as ADD Volume=ΣC_{X}, C_{Y} (I_{data}−I_{Loewe}), where I_{Loewe}(C_{X}, C_{Y}) is the inhibition that satisfies (C_{X}/EC_{X})+(C_{Y}/EC_{Y})=1, and EC_{X,Y} are the effective concentrations at I_{Loewe} for the single agent curves. A “Synergy Score” was also used, where the Synergy Score S=log f_{X} log f_{Y} ΣI_{data}(I_{data}−I_{Loewe}), summed over all non-single-agent concentration pairs, and where log f_{X,Y} is the natural logarithm of the dilution factors used for each single agent. This effectively calculates a volume between the measured and Loewe additive response surfaces, weighted towards high inhibition and corrected for varying dilution factors. An uncertainty σ_{S} was calculated for each synergy score, based on the measured errors for the I_{data} values and standard error propagation.

### Example 2

The MM.1S, EJM, RPMI-8226, INA-6, ANBL-6 and OCI-ly10 cell lines were used to examine the activity of various BAR agonists in combination with antiproliferative compounds that have been deployed to treat B-cell malignancies. Synergy scores are provided followed by representative data from some of the combination matrix analysis.

Potent combination synergistic anti-proliferative activities are observed for multiple myeloma, DLBCL and Burkitt's lymphoma cells when the beta 2 adrenergic agonists salmeterol, formoterol, terbutaline, isoetharine, ritodrine, salbutamol, clenbuterol, fenoterol, metaproterenol, levalbuterol, isoproterenol, pirbuterol, broxaterol, picumeterol, or procaterol are used in combination with the glucocorticoid dexamethasone (Table 10).

TABLE 10
Summary of Synergy Scores for BAR Agonists that Synergize with
Dexamethasone in One or More B-cell Malignancy Cell Lines
(MM.1S, EJM, RPMI-8226, ANBL-6, and OCI-ly10)

| | | | RPMI- | | OCI- | |
|---|---|---|---|---|---|---|
| Combination | MM.1S | EJM | 8226 | ANBL-6 | ly10 | GA-10 |
| formoterol × dexamethasone | 6.74 | n.d. | 2.83 | 6.26 | n.d. | n.d. |
| salmeterol × dexamethasone | 18.81 | 9.01 | 1.65 | 11.34 | 9.80 | 1.87 |
| terbutaline × dexamethasone | 6.02 | 3.19 | n.d. | n.d. | n.d. | n.d. |
| isoetharine × dexamethasone | 5.38 | 5.14 | n.d. | n.d. | n.d. | n.d. |
| ritodrine × dexamethasone | 5.71 | 5.13 | n.d. | n.d. | n.d. | n.d. |
| salbutamol × dexamethasone | 6.19 | 4.27 | n.d. | n.d. | n.d. | n.d. |
| clenbuterol × dexamethasone | 9.54 | 5.08 | 6.61 | 6.67 | n.d. | n.d. |
| fenoterol × dexamethasone | 4.73 | 2.09 | 2.52 | n.d. | n.d. | n.d. |
| metaproterenol × dexamethasone | 6.10 | 3.81 | n.d. | n.d. | n.d. | n.d. |
| levalbuterol × dexamethasone | 11.49 | 5.40 | n.d. | n.d. | n.d. | n.d. |
| isoproterenol × dexamethasone | 10.81 | n.d. | n.d. | n.d. | n.d. | n.d. |
| pirbuterol × dexamethasone | 9.09 | n.d. | n.d. | n.d. | n.d. | n.d. |
| broxaterol × dexamethasone | 10.67 | n.d. | n.d. | n.d. | n.d. | n.d. |
| picumeterol × dexamethasone | 5.61 | n.d. | n.d. | n.d. | n.d. | n.d. |
| procaterol × dexamethasone | 10.61 | n.d. | n.d. | n.d. | n.d. | n.d. |

Below are representative high resolution 9×9 data where BAR agonists were used in combination with dexamethasone. (Tables 11-20).

TABLE 11
Antiproliferative Activity of Dexamethasone (DEX) and Formoterol against Human
Multiple Myeloma Cells (MM.1S)

| | Formoterol (μM) | | | | | | | | |
|---|---|---|---|---|---|---|---|---|---|
| DEX (μM) | 0.00032 | 0.00016 | 0.000081 | 0.0000405 | 0.0000202 | 0.0000101 | 0.00000506 | 0.00000253 | 0 |
| 2.03 | 94 | 93.7 | 93 | 93.8 | 93 | 93.7 | 93.2 | 92 | 85.5 |
| 0.675 | 93.6 | 93.2 | 92.7 | 93.6 | 93.8 | 93.1 | 93.4 | 92.4 | 84.5 |
| 0.225 | 93.4 | 93.9 | 93.3 | 93.7 | 93.6 | 92.4 | 93.4 | 89.5 | 81.8 |
| 0.075 | 93.1 | 94.1 | 93.3 | 93.7 | 93.4 | 93.3 | 92.2 | 89 | 78.3 |
| 0.025 | 93.1 | 93.2 | 93.3 | 92.5 | 92.1 | 92.5 | 90.2 | 87.2 | 65.7 |
| 0.00834 | 93 | 92.2 | 92.8 | 92.5 | 90.7 | 82.5 | 69.2 | 56.9 | 39 |
| 0.00278 | 91.8 | 90.7 | 89.3 | 93.6 | 81.5 | 71.7 | 60.5 | 49.5 | 25 |
| 0.00093 | 86.7 | 82.5 | 78.6 | 77.2 | 71.7 | 55.6 | 43.4 | 27.5 | 9.24 |
| 0 | 72.5 | 68.7 | 65.9 | 54.7 | 52.7 | 40.8 | 23.8 | 18.9 | 8.95 |

TABLE 12
Antiproliferative Activity of Dexamethasone (DEX) and Formoterol against
Human Multiple Myeloma Cells (ANBL-6)

| | Formoterol (μM) | | | | | | | | |
|---|---|---|---|---|---|---|---|---|---|
| DEX (μM) | 0.00032 | 0.00016 | 0.000081 | 0.0000405 | 0.0000202 | 0.0000101 | 0.00000506 | 0.00000253 | 0 |
| 2.03 | 90.5 | 87.8 | 93.2 | 81.2 | 73 | 64.2 | 49.8 | 37.7 | 23.4 |
| 0.675 | 90.2 | 87.9 | 85.6 | 79.1 | 71.8 | 61.7 | 47.3 | 38.9 | 22.5 |
| 0.225 | 90.6 | 86 | 86.1 | 81.7 | 71.1 | 53.1 | 40.6 | 28.4 | 12 |
| 0.075 | 87.8 | 86.6 | 83.6 | 76.2 | 66.8 | 51.8 | 44.2 | 32.6 | 14.7 |
| 0.025 | 87.7 | 80.7 | 78.6 | 72.3 | 64.9 | 53 | 41.1 | 38.2 | 6.97 |
| 0.00834 | 79.5 | 78.1 | 73.6 | 63.5 | 54.3 | 34.7 | 26 | 13.7 | 6.13 |
| 0.00278 | 73.2 | 66.3 | 64.7 | 57.9 | 45.8 | 40.2 | 32 | 18.7 | −3.92 |
| 0.00093 | 68.9 | 60.1 | 56.7 | 51.6 | 43.7 | 30.9 | 26.5 | 17.6 | 5.06 |
| 0 | 61.9 | 61 | 55.4 | 48.8 | 39.9 | 30 | 26.7 | 23.3 | 6.3 |

TABLE 13
Antiproliferative Activity of Dexamethasone (DEX) and Formoterol against
Human Multiple Myeloma Cells (RPMI-8226)

| | Formoterol (μM) | | | | | | | | |
|---|---|---|---|---|---|---|---|---|---|
| DEX (μM) | 0.00032 | 0.00016 | 0.000081 | 0.0000405 | 0.0000202 | 0.0000101 | 0.00000506 | 0.00000253 | 0 |
| 2.03 | 53.8 | 55 | 51.9 | 50.2 | 54.7 | 47.7 | 41.7 | 36.9 | 39.5 |
| 0.675 | 54.4 | 53 | 52.1 | 47.5 | 47.8 | 46.9 | 43.6 | 41.3 | 27.8 |
| 0.225 | 55.7 | 53.3 | 50.3 | 52.6 | 49.4 | 46.6 | 39.1 | 36.4 | 28 |
| 0.075 | 43.1 | 54 | 51.3 | 45 | 47.1 | 45.8 | 38.8 | 37.3 | 27.7 |
| 0.025 | 43.9 | 45.3 | 42.2 | 41.2 | 40.7 | 39.2 | 32.5 | 29 | 18.4 |
| 0.00834 | 32.8 | 31.9 | 31.1 | 33.1 | 28.2 | 27.8 | 19 | 16.4 | 9.55 |
| 0.00278 | 26.3 | 23.9 | 26.7 | 25.1 | 23.2 | 16.4 | 14.1 | 14.8 | 12.3 |
| 0.00093 | 14.2 | 11.3 | 15.7 | 14.5 | 15.9 | 10.1 | 16.3 | 11 | 10.9 |
| 0 | 15.4 | 11.6 | 10.5 | 4.33 | 5.4 | 11.4 | 10.2 | 11.9 | 5.75 |

TABLE 14
Antiproliferative Activity of Dexamethasone (DEX) and Isoetharine against
Human Multiple Myeloma Cells (EJM)

| | Isoetharine (μM) | | | | | | | | |
|---|---|---|---|---|---|---|---|---|---|
| DEX (μM) | 0.0203 | 0.0101 | 0.00506 | 0.00253 | 0.00127 | 0.000633 | 0.000316 | 0.000158 | 0 |
| 2.03 | 73.4 | 57.4 | 53.2 | 46.8 | 33.1 | 32 | 29 | 11.8 | 5.97 |
| 0.675 | 75.2 | 56.7 | 44.3 | 47.6 | 39.4 | 36 | 22 | 9.68 | −0.146 |
| 0.225 | 71.5 | 51.5 | 55.6 | 47.1 | 40.1 | 25.1 | 23.5 | 8.15 | 2.33 |
| 0.075 | 72.9 | 55.7 | 51.6 | 42.4 | 34.7 | 30.7 | 14.6 | 12.7 | −5.53 |
| 0.025 | 66.3 | 50.9 | 45.4 | 44.5 | 30.4 | 16.4 | 21 | 4.59 | 5.46 |
| 0.00834 | 58.8 | 48.2 | 33.8 | 35.3 | 27.1 | 24.6 | −12.4 | −2.33 | −10.8 |
| 0.00278 | 53.5 | 24.5 | 33.6 | 27.1 | 20 | 0.291 | 17.5 | 4.95 | −3.42 |
| 0.00093 | 46.8 | 37 | 27.7 | 28.6 | 16.4 | 16.7 | 5.17 | 5.6 | −5.82 |
| 0 | 41.4 | 21.8 | 29 | 23.6 | 19.5 | −0.146 | 7.35 | 7.35 | −1.24 |

TABLE 15
Antiproliferative Activity of Dexamethasone (DEX) and Isoetharine against
Human Multiple Myeloma Cells (MM.1S)

| | Isoetharine (μM) | | | | | | | | |
|---|---|---|---|---|---|---|---|---|---|
| DEX (μM) | 0.0203 | 0.0101 | 0.00506 | 0.00253 | 0.00127 | 0.000633 | 0.000316 | 0.000158 | 0 |
| 2.03 | 95.4 | 93.8 | 93.1 | 92.6 | 89.6 | 80 | 81.1 | 69 | 72.5 |
| 0.675 | 95.2 | 94.6 | 91.5 | 92 | 88.1 | 86.9 | 78.7 | 78 | 64.8 |
| 0.225 | 95.6 | 93.2 | 94 | 91.6 | 90.8 | 77.4 | 78.7 | 68.9 | 62.5 |
| 0.075 | 94.9 | 92.5 | 91.4 | 91.6 | 86.6 | 76.7 | 66.5 | 68.6 | 56.4 |
| 0.025 | 94.4 | 86.4 | 86.3 | 78.7 | 73.3 | 50 | 63.1 | 50.7 | 45.5 |
| 0.00834 | 86.7 | 79 | 70.3 | 75.2 | 58.4 | 53.7 | 24.6 | 31.8 | 25.6 |
| 0.00278 | 81.4 | 67.9 | 64.8 | 42.6 | 45 | 19.2 | 26.1 | 18.6 | 17.9 |
| 0.00093 | 74.5 | 49.9 | 41.8 | 52.4 | 31.9 | 36.2 | 3.38 | 12.7 | 1.52 |
| 0 | 53.9 | 18 | 29.5 | 14.7 | 17.7 | 0.225 | 18.5 | 9.23 | −2.19 |

TABLE 16
Antiproliferative Activity of Dexamethasone (DEX) and Salmeterol against
Human Multiple Myeloma Cells (EJM)

| | Salmeterol (μM) | | | | | | | | |
|---|---|---|---|---|---|---|---|---|---|
| DEX (μM) | 0.0203 | 0.0101 | 0.00506 | 0.00253 | 0.00127 | 0.000633 | 0.000316 | 0.000158 | 0 |
| 2.03 | 77.2 | 75.6 | 77.9 | 74.9 | 75 | 73.3 | 76.2 | 74.9 | 10.1 |
| 0.675 | 79.1 | 78.6 | 74.7 | 77 | 76 | 78.3 | 73.8 | 75.5 | 4.03 |
| 0.225 | 77.4 | 76.9 | 77.8 | 74.6 | 76 | 74.7 | 78.1 | 75.2 | 7.36 |
| 0.075 | 79.5 | 77.7 | 73.7 | 75.5 | 75.4 | 78 | 71.5 | 73.6 | −0.778 |
| 0.025 | 76.2 | 72.2 | 73.8 | 72.1 | 73.8 | 69.6 | 77.4 | 66.5 | 7.43 |
| 0.00834 | 69.4 | 69.7 | 68.7 | 66 | 63.8 | 68.6 | 58.9 | 64.1 | −13 |
| 0.00278 | 61.4 | 54 | 62 | 56.3 | 57.1 | 49.7 | 60.7 | 55.4 | 4.1 |
| 0.00093 | 57.4 | 55 | 51.7 | 51.2 | 51.2 | 57.4 | 44.2 | 40.9 | −18.7 |
| 0 | 43.6 | 42.8 | 47.9 | 43.2 | 45.4 | 39.4 | 47.7 | 38.1 | 10 |

TABLE 17
Antiproliferative Activity of Dexamethasone (DEX) and Salmeterol against
Human Multiple Myeloma Cells (MM.1S)

| | Salmeterol (μM) | | | | | | | | |
|---|---|---|---|---|---|---|---|---|---|
| DEX (μM) | 0.00032 | 0.00016 | 0.000081 | 0.0000405 | 0.0000202 | 0.0000101 | 0.00000506 | 0.00000253 | 0 |
| 2.03 | 90.1 | 89.5 | 90.2 | 89.5 | 91 | 89.6 | 90.7 | 90.7 | 33.4 |
| 0.675 | 88.6 | 88.5 | 87.8 | 88.2 | 87.6 | 89 | 87.9 | 85.8 | 21.7 |
| 0.225 | 87.9 | 87.7 | 87.8 | 86.6 | 87.5 | 87.8 | 89.3 | 87.5 | 25.6 |
| 0.075 | 85.2 | 83.7 | 85.9 | 83.9 | 85 | 83.5 | 84 | 83.7 | 29.9 |
| 0.025 | 80.1 | 81.9 | 82.9 | 80 | 76.9 | 78.2 | 77.5 | 76.5 | 20.8 |
| 0.00834 | 72.9 | 74 | 72.6 | 69.2 | 66.2 | 74.8 | 69.8 | 68 | 1.81 |
| 0.00278 | 69.4 | 66.1 | 67 | 65.8 | 66.2 | 62.3 | 65.4 | 60.7 | 4.93 |
| 0.00093 | 60 | 60.2 | 58.7 | 60.5 | 61.1 | 62.1 | 60.3 | 60.2 | 6.94 |
| 0 | 59.6 | 58.1 | 55.9 | 57.8 | 55.1 | 58.1 | 59.7 | 52.9 | 2.79 |

TABLE 18
Antiproliferative Activity of Dexamethasone (DEX) and Salmeterol against
Human Multiple Myeloma Cells (ANBL-6)

| | Salmeterol (μM) | | | | | | | | |
|---|---|---|---|---|---|---|---|---|---|
| DEX (μM) | 0.00032 | 0.00016 | 0.000081 | 0.0000405 | 0.0000202 | 0.0000101 | 0.00000506 | 0.00000253 | 0 |
| 2.03 | 95 | 94.6 | 94.4 | 94.2 | 94.3 | 93.9 | 90.2 | 88 | 79.2 |
| 0.675 | 94.4 | 94.8 | 94.8 | 94.6 | 93.6 | 94.3 | 91.7 | 89.1 | 78 |
| 0.225 | 94.1 | 94.5 | 95 | 94.6 | 93.8 | 92.5 | 90.2 | 86 | 76.9 |
| 0.075 | 93.8 | 94.4 | 95.1 | 94 | 93.1 | 91.2 | 86.2 | 81.1 | 72.3 |
| 0.025 | 94.1 | 94 | 94.8 | 93.8 | 90.2 | 83.9 | 74.8 | 68 | 58.1 |
| 0.00834 | 93 | 88.6 | 89.6 | 85.8 | 78.5 | 66.9 | 50.4 | 51.1 | 29.9 |
| 0.00278 | 85.8 | 79.4 | 79.3 | 73.5 | 60 | 48.2 | 38.5 | 26.5 | 15.4 |
| 0.00093 | 73.6 | 59.2 | 56.1 | 59.7 | 41 | 43.8 | 24.2 | 19.5 | 9.36 |
| 0 | 56.1 | 46.3 | 41.9 | 36.1 | 22.1 | 24.2 | 18.6 | 13.4 | 4.07 |

TABLE 19
Antiproliferative Activity of Dexamethasone (DEX) and Terbutaline against
Human Multiple Myeloma Cells (MM.1S)

| | Terbutaline (μM) | | | | | | | | |
|---|---|---|---|---|---|---|---|---|---|
| DEX (μM) | 2.03 | 1.01 | 0.506 | 0.253 | 0.127 | 0.0633 | 0.0316 | 0.0158 | 0 |
| 2.03 | 96 | 96.1 | 95.6 | 95.2 | 95.1 | 94.5 | 94.6 | 93.7 | 73.4 |
| 0.675 | 95.2 | 95.3 | 94.8 | 95.2 | 95.3 | 94.9 | 94.7 | 94.8 | 71.2 |
| 0.225 | 95.2 | 95 | 95.4 | 95.4 | 94.6 | 95 | 94.4 | 93.7 | 70.1 |
| 0.075 | 95.2 | 95.4 | 94.8 | 95.2 | 94.6 | 94.9 | 94 | 91.5 | 63.4 |
| 0.025 | 95.4 | 94.8 | 94.8 | 94.5 | 94.7 | 94 | 92.7 | 90.7 | 52.6 |
| 0.00834 | 92.5 | 93.3 | 92.6 | 91.2 | 89.3 | 88.5 | 85.5 | 82.7 | 30.5 |
| 0.00278 | 85.4 | 81.9 | 83.7 | 89.9 | 76.9 | 71.8 | 65.8 | 61.9 | 17.3 |
| 0.00093 | 72.1 | 72.3 | 70 | 67.3 | 65.3 | 56.8 | 57.8 | 48.7 | 15 |
| 0 | 58 | 55.2 | 53 | 51.1 | 40 | 35.7 | 38.4 | 30 | 11.5 |

TABLE 20
Antiproliferative Activity of Dexamethasone (DEX) and Salmeterol against
Diffuse Large B-Cell Lymphoma Cell Line OCI-Ly10

| | Salmeterol (μM) | | | | | | | | |
|---|---|---|---|---|---|---|---|---|---|
| DEX (μM) | 0.769 | 0.256333 | 0.085444 | 0.028481 | 0.009494 | 0.003165 | 0.001055 | 0.000352 | 0 |
| 0.101 | 100 | 96 | 91 | 80 | 74 | 71 | 69 | 71 | 67 |
| 0.033666667 | 100 | 95 | 86 | 62 | 62 | 56 | 59 | 52 | 56 |
| 0.011222222 | 97 | 87 | 57 | 42 | 40 | 41 | 41 | 45 | 29 |
| 0.003740741 | 90 | 63 | 40 | 27 | 29 | 21 | 27 | 22 | 22 |
| 0.001246914 | 69 | 38 | 21 | 14 | 7.5 | 10 | 9.4 | 8.9 | 9.7 |
| 0.000415638 | 64 | 33 | 22 | 19 | 7.6 | 11 | 6.7 | 12 | 4.5 |
| 0.000138546 | 50 | 33 | 13 | 5.5 | 1.7 | 16 | 13 | 6.2 | 0.78 |
| 0.000046182 | 57 | 36 | 21 | 11 | 14 | 0.6 | 16 | 9.7 | 12 |
| 0 | 71 | 27 | 6.3 | −0.6 | 9.1 | 6.3 | 5.9 | 16 | 0.07 |

Strong synergy is also observed when beta adrenergic agonists are combined with either lenalidomide (Table 21), melphalan (Table 22), or doxorubicin (Table 23), three drugs that are part of standard of care for the treatment of multiple myeloma.

TABLE 21
Summary of Synergy Scores for the BAR Agonists Salmeterol,
Formoterol, Clenbuterol, Salbutamol, and Terbutaline when
Combined with Lenalidomide in the Multiple Myeloma Cell Lines
MM.1S, ANBL-6, and MM.1R

| Combination | MM.1S | ANBL-6 | MM.1R |
|---|---|---|---|
| salmeterol × lenalidomide | 4.62 | 5.01 | n.d. |
| formoterol × lenalidomide | 5.01 | 5.27 | n.d. |
| clenbuterol × lenalidomide | n.d. | 5.50 | 4.86 |
| salbutamol × lenalidomide | n.d. | 3.76 | 2.74 |
| terbutaline × lenalidomide | 0.97 | 5.39 | 4.22 |
| broxaterol × lenalidomide | 3.94 | n.d. | n.d. |
| picumeterol × lenalidomide | 2.64 | n.d. | n.d. |

TABLE 22
Summary of Synergy Scores for the BAR Agonists Salmeterol,
Clenbuterol, Salbutamol, and Terbutaline when Combined with
Melphalan in the Multiple Myeloma Cell Lines ANBL-6, EJM,
INA-6, and MM.1R

| Combination | ANBL-6 | EJM | INA-6 | MM.1R |
|---|---|---|---|---|
| salmeterol × melphalan | n.d. | 2.26 | 1.19 | 4.21 |
| clenbuterol × melphalan | n.d. | n.d. | 1.76 | 2.46 |
| salbutamol × melphalan | 3.36 | 1.34 | n.d. | n.d. |
| terbutaline × melphalan | 3.84 | 1.22 | 1.88 | 1.81 |

TABLE 23
Summary of Synergy Scores for the BAR Agonists Salmeterol and
Terbutaline when Combined with Doxorubicin in the Multiple
Myeloma Cell Lines MM.1S, EJM, ANBL-6, and MM.1R

| Combination | MM.1S | EJM | ANBL-6 | MM.1R |
|---|---|---|---|---|
| salmeterol × doxorubicin | 1.73 | 2.11 | 5.67 | 1.13 |
| terbutaline × doxorubicin | 1.23 | 0.76 | 3.28 | 0.84 |

Representative 9×9 data for BAR agonists in combination with lenalidomide (Tables 24-25), melphalan (Table 26), and doxorubicin (Table 27) are shown below.

TABLE 24
Antiproliferative Activity of Lenalidomide (LEN) and Salmeterol against
Human Multiple Myeloma Cells (MM.1S)

| | Salmeterol (μM) | | | | | | | | |
|---|---|---|---|---|---|---|---|---|---|
| LEN (μM) | 0.32 | 0.16 | 0.081 | 0.0405 | 0.0202 | 0.0101 | 0.00506 | 0.00000253 | 0 |
| 4.04 | 88.7 | 84.5 | 85.5 | 78.1 | 75.1 | 70.3 | 61 | 57.4 | 50.2 |
| 2.02 | 85.3 | 81.4 | 79.6 | 72.8 | 65.7 | 64.7 | 55.7 | 47.9 | 38.2 |
| 1.01 | 80.2 | 80 | 77.3 | 73 | 67.9 | 60.7 | 51.6 | 49.1 | 36.3 |
| 0.253 | 80.8 | 77.9 | 76.3 | 69.1 | 60.1 | 53.6 | 44.4 | 40.1 | 36.7 |
| 0.126 | 81.2 | 73.6 | 73.9 | 69.4 | 59.9 | 54.5 | 42.9 | 37.3 | 31.2 |
| 0.0632 | 75.7 | 74.1 | 70.2 | 66 | 61.4 | 49.8 | 40.5 | 36.8 | 29 |
| 0.0316 | 75.6 | 71.3 | 66.9 | 67 | 55.5 | 52.5 | 45.5 | 32.8 | 27.3 |
| 0 | 74 | 67.1 | 61.4 | 59.9 | 55.8 | 46.5 | 41.5 | 34.4 | 26.4 |
| 4.04 | 56.5 | 44.7 | 43.7 | 35.2 | 22.1 | 22.6 | 19.2 | 15.1 | 3.71 |

TABLE 25
Antiproliferative Activity of Lenalidomide (LEN) and Salmeterol against
Human Multiple Myeloma Cells (ANBL-6)

| | Salmeterol (μM) | | | | | | | | |
|---|---|---|---|---|---|---|---|---|---|
| LEN (μM) | 0.00032 | 0.00016 | 0.000081 | 0.0000405 | 0.0000202 | 0.0000101 | 0.00000506 | 0.00000253 | 0 |
| 4.04 | 90.7 | 87.3 | 87.7 | 82.7 | 78.5 | 77.1 | 69.9 | 65 | 61.9 |
| 2.02 | 87.6 | 83.7 | 84.6 | 81.4 | 78.5 | 72.6 | 68 | 64.2 | 52.8 |
| 1.01 | 84.7 | 86.7 | 79.6 | 78.7 | 76.4 | 71.4 | 67.8 | 61.8 | 54.3 |
| 0.505 | 87.3 | 84 | 81.5 | 79.3 | 73.8 | 67.8 | 65.8 | 60.4 | 53.5 |
| 0.253 | 87.4 | 82.3 | 79.1 | 79.8 | 74.8 | 68.5 | 65.2 | 58.2 | 51.8 |
| 0.126 | 84.9 | 80.9 | 78.3 | 75.6 | 74.5 | 67 | 61.4 | 62.1 | 52.5 |
| 0.0632 | 85 | 80.3 | 81.9 | 73.3 | 75.2 | 67.6 | 63.9 | 57.4 | 44.8 |
| 0.0316 | 83.2 | 74.6 | 73.5 | 72 | 70.5 | 61.7 | 60.6 | 54.6 | 34.3 |
| 0 | 55.7 | 52.9 | 46.7 | 45.9 | 39 | 33.9 | 34.7 | 21.5 | −3.58 |

TABLE 26
Antiproliferative Activity of Melphalan (MELPH) and Salmeterol against
Human Multiple Myeloma Cells (MM.1S)

| | Salmeterol (μM) | | | | | | | | |
|---|---|---|---|---|---|---|---|---|---|
| MELPH (μM) | 0.00032 | 0.00016 | 0.000081 | 0.0000405 | 0.0000202 | 0.0000101 | 0.00000506 | 0.00000253 | 0 |
| 20.9 | 92 | 93 | 92.5 | 92.2 | 92.7 | 92.8 | 92.4 | 92.3 | 93.3 |
| 10.5 | 90.9 | 90.6 | 89.5 | 88 | 87.3 | 87.4 | 83.2 | 84.2 | 79.6 |
| 5.23 | 89.3 | 89.2 | 91 | 88.6 | 83.9 | 85.8 | 81.9 | 87.2 | 74.5 |
| 2.61 | 87.3 | 83.4 | 85.8 | 84.5 | 79.6 | 78.4 | 78.7 | 72.7 | 62 |
| 1.31 | 80.8 | 75.4 | 74.6 | 67.9 | 71 | 60.5 | 63.3 | 58.5 | 52.7 |
| 0.653 | 72.8 | 64.9 | 63.3 | 56.6 | 57 | 45.7 | 43.7 | 42.1 | 32.1 |
| 0.327 | 67.2 | 62.2 | 55.2 | 54 | 39.2 | 33.6 | 34.3 | 21.4 | 23 |
| 0.163 | 64.8 | 60.4 | 46.7 | 48.4 | 37.9 | 33.3 | 21.7 | 18 | 8.01 |
| 0 | 64.5 | 47.2 | 43.1 | 40.7 | 30.9 | 25.9 | 20.8 | 15.3 | 2.99 |

TABLE 27
Antiproliferative Activity of Doxorubicin (DOX) and Salmeterol against
Human Multiple Myeloma Cells (MM.1S)

| | Salmeterol (μM) | | | | | | | | |
|---|---|---|---|---|---|---|---|---|---|
| DOX (μM) | 0.00032 | 0.00016 | 0.000081 | 0.0000405 | 0.0000202 | 0.0000101 | 0.00000506 | 0.00000253 | 0 |
| 2.06 | 94.2 | 93.7 | 94.9 | 94.3 | 94.5 | 94.6 | 94 | 94.1 | 94.5 |
| 0.688 | 90.2 | 89.6 | 89.4 | 90.8 | 92 | 92.3 | 91.9 | 91.3 | 92.8 |
| 0.229 | 76.7 | 76.1 | 75.2 | 76 | 75.3 | 77.8 | 77.2 | 78.2 | 76.7 |
| 0.0764 | 77.7 | 79.5 | 77.5 | 79 | 77.6 | 78.8 | 77.3 | 77.4 | 74 |
| 0.0255 | 79.8 | 77.1 | 76.5 | 76.9 | 74.7 | 76.3 | 67.5 | 72.6 | 61.2 |
| 0.00849 | 65.4 | 49.7 | 55.9 | 50.4 | 44.4 | 47.9 | 20.3 | 19.5 | 2.23 |
| 0.00283 | 58 | 46.3 | 52.5 | 43.1 | 31.8 | 24.3 | 13.9 | 8.19 | 1.3 |
| 0.000943 | 55.3 | 52.8 | 41.2 | 39.3 | 33.3 | 30.6 | 9.49 | 9.3 | 2.23 |
| 0 | 58.7 | 44.3 | 36.4 | 32.5 | 26.4 | 16.5 | 16 | 14.9 | −2.79 |

Synergistic combination activity was also observed when BAR agonists were used in combination with the proteosome inhibitor bortezomib (Velcade). High resolution 9×9 combination analysis of formoterol and salmeterol crossed with bortezomib in the MM.1S multiple myeloma cell line is shown in Tables 28 and 29. As seen in Table 28, the single agent activity for 0.00253 μM bortezomib is 30.8% inhibition of proliferation, and the single agent activity of 0.0000202 μM formoterol is 51%. Combination anti-proliferative activity is 78%, a value that is more than additive for combined activity of the single agents. Similar combination effects are seen with salmeterol (Table 29). For example, the single agent activity for 0.00253 nM bortezomib is 19.6%, and single agent activity for 0.00032 μM salmeterol is 42%. Combination anti-proliferative activity is 82.6%.

TABLE 28
Antiproliferative Activity of Bortezomib (BORT) and Formoterol against
Human Multiple Myeloma Cells (MM.1S)

| | Formoterol (μM) | | | | | | | | |
|---|---|---|---|---|---|---|---|---|---|
| BORT (μM) | 0.00032 | 0.00016 | 0.000081 | 0.0000405 | 0.0000202 | 0.0000101 | 0.00000506 | 0.00000253 | 0 |
| 0.0405 | 79.2 | 87.2 | 85.5 | 82.8 | 93.8 | 94.1 | 93.3 | 93.4 | 0 |
| 0.0203 | 87.7 | 93.1 | 93.8 | 93.6 | 92.9 | 94.2 | 93.6 | 93 | 94.6 |
| 0.0101 | 92.3 | 93.8 | 92.8 | 93.7 | 92.8 | 92.2 | 90.3 | 91.9 | 94.4 |
| 0.00506 | 92.5 | 93 | 92.3 | 91.4 | 89.2 | 78.8 | 86.4 | 80.8 | 91.8 |
| 0.00253 | 90.8 | 88.8 | 83.3 | 78.3 | 78 | 64.7 | 59.6 | 49.1 | 30.8 |
| 0.00127 | 81.6 | 74.8 | 78.2 | 69.2 | 56.1 | 43.2 | 27.9 | 19.5 | 7.32 |
| 0.000633 | 77.8 | 70 | 72.5 | 65.1 | 58.8 | 46.3 | 33 | 20.3 | 5.74 |
| 0.000316 | 75.5 | 69.1 | 68.5 | 65 | 56.8 | 45 | 37 | 19.5 | 6.04 |
| 0 | 72.7 | 69.4 | 65.7 | 57.5 | 51 | 40.7 | 36.2 | 22.4 | 5.51 |

TABLE 29
Antiproliferative Activity of Bortezomib (BORT) and Salmeterol against
Human Multiple Myeloma Cells (MM.1S)

| | Salmeterol (μM) | | | | | | | | |
|---|---|---|---|---|---|---|---|---|---|
| BORT (μM) | 0.00032 | 0.00016 | 0.000081 | 0.0000405 | 0.0000202 | 0.0000101 | 0.00000506 | 0.00000253 | 0 |
| 0.0405 | | 94.5 | 93.9 | 94.1 | 94.4 | 94.3 | 94.3 | 94.4 | 93.5 |
| 0.0203 | 94.2 | 94.3 | 94.1 | 93.7 | 93.5 | 93.7 | 93.7 | 94.9 | 94.7 |
| 0.0101 | 94.2 | 94 | 93.1 | 94.1 | 93.5 | 92.9 | 91.6 | 92.1 | 94 |
| 0.00506 | 92 | 88.1 | 88.7 | 83.8 | 78.9 | 71.6 | 78.2 | 54.3 | 91.6 |
| 0.00253 | 82.6 | 77.5 | 62.2 | 54.4 | 53.8 | 38.9 | 35.2 | 24.4 | 19.6 |
| 0.00127 | 61.7 | 43.2 | 43.2 | 30.4 | 26.3 | 16.5 | 14.9 | 4.92 | 2.2 |
| 0.000633 | 53.7 | 43.3 | 35.5 | 31.5 | 28.7 | 19.8 | 14.4 | 7.22 | 2.43 |
| 0.000316 | 52.7 | 38.5 | 32.4 | 29.9 | 23 | 18 | 9.37 | 2.56 | 1.9 |
| 0 | 42.1 | 35.4 | 31 | 26.5 | 21.9 | 16.9 | 16.9 | 8.95 | −1.02 |

BAR agonists also had potent synergistic combination activity against multiple B-cell malignancies when used in combination with adenosine receptor A2A agonists (multiple myeloma, DLBCL, and Burkitt's lymphoma) or phosphodiesterase inhibitors (multiple myeloma and Burkitt's lymphoma). Representative synergy scores are shown in Table 30 and representative high resolution 9×9 combination analysis are shown in Table 31 (the adenosine receptor A2A agonist ATL 1222 crossed with Salmeterol using the multiple myeloma cell line MM.1S) and Tables 32-33 (Salmeterol combined with the PDE inhibitor trequinsin using the MM.1S and ANBL-6 multiple myeloma cell lines).

TABLE 30
Summary of Synergy Scores for the BAR Agonists when Combined
with Adenosine Receptor Agonists ATL 1222 and Chloro-IB-MECA
or the PDE Inhibitors Trequinsin and Roflumilast in MM.1S and
ANBL-6 (Multiple Myeloma), GA-10 (Burkitt's Lymphoma), or
OCI-ly10 (DLBCL)

| | | GA- | | |
|---|---|---|---|---|
| Combination | MM.1S | 10 | ANBL-6 | OCI-ly10 |
| salmeterol × ATL 1222 | 4.88 | n.d. | 1.47 | 1.36 |
| formoterol × ATL 1222 | 2.09 | n.d. | 1.86 | n.d. |
| salmeterol × chloro-IB-MECA | n.d. | 1.07 | n.d. | 3.26 |
| salmeterol × trequinsin | 10.4 | n.d. | 6.84 | 2.99 |
| formoterol × trequinsin | 6.34 | n.d. | n.d. | n.d. |
| salmeterol × roflumilast | n.d. | n.d. | n.d. | 4.28 |

TABLE 31
Antiproliferative Activity of ATL 1222 and Salmeterol against
Human Multiple Myeloma Cells (MM.1S)

| | Salmeterol (μM) | | | | | | | | |
|---|---|---|---|---|---|---|---|---|---|
| ATL 1222 (μM) | 0.00032 | 0.00016 | 0.000081 | 0.0000405 | 0.0000202 | 0.0000101 | 0.00000506 | 0.00000253 | 0 |
| 0.0203 | 84.7 | 82.3 | 81.8 | 77.3 | 78.3 | 76.5 | 70.8 | 73.8 | 70.1 |
| 0.0101 | 82.9 | 81.5 | 79.6 | 77.6 | 77.2 | 73 | 72.5 | 71.8 | 65.9 |
| 0.00506 | 81.1 | 79 | 77.1 | 71.8 | 72.4 | 69.2 | 67.6 | 65.8 | 60.8 |
| 0.00253 | 78.9 | 73.6 | 72.6 | 71.2 | 65.2 | 60.9 | 58.4 | 58.3 | 48.6 |
| 0.00127 | 75.7 | 64.8 | 66.3 | 61.1 | 59.2 | 55.1 | 47.6 | 45.2 | 39.2 |
| 0.000633 | 68.7 | 62 | 56.4 | 53.8 | 45.4 | 44.2 | 36.7 | 28.8 | 27.9 |
| 0.000316 | 65.6 | 53.8 | 57 | 49.5 | 41 | 33 | 25.3 | 17.3 | 13 |
| 0.000158 | 59.8 | 50.1 | 46.2 | 42.9 | 33.5 | 32.6 | 18.9 | 7.42 | 8.7 |
| 0 | 52 | 36.6 | 34.8 | 26.3 | 20 | 16.6 | 12.2 | 10.9 | −1.96 |

TABLE 32
Antiproliferative Activity of Salmeterol (SAL) and Trequinsin against
Human Multiple Myeloma Cells (MM.1S)

| | Trequinsin (μM) | | | | | | | | |
|---|---|---|---|---|---|---|---|---|---|
| SAL (μM) | 10.1 | 3.38 | 1.13 | 0.375 | 0.125 | 0.0417 | 0.0139 | 0.00464 | 0 |
| 0.000324 | 90.4 | 83.3 | 77.9 | 76.9 | 76.5 | 71.2 | 72.6 | 60.1 | 54 |
| 0.000162 | 90.8 | 77.5 | 74.1 | 73.6 | 64.5 | 64 | 61.5 | 54 | 46.5 |
| 0.000081 | 84.3 | 77.6 | 73.6 | 56.8 | 60.1 | 52.7 | 54.5 | 42 | 45.9 |
| 0.0000405 | 78.3 | 75.6 | 61.9 | 63.9 | 58.8 | 53.2 | 39.6 | 42.4 | 35.5 |
| 0.0000202 | 84.1 | 63.8 | 63.5 | 55.9 | 42.6 | 44.9 | 42.6 | 26.5 | 32.6 |
| 0.0000101 | 76.1 | 55.5 | 39.4 | 28.6 | 33.4 | 28.8 | 23.7 | 21 | 21.3 |
| 0.00000506 | 69.5 | 40.3 | 29.7 | 24.4 | 16.9 | 15.9 | 18.7 | 20 | 16.3 |
| 0.00000253 | 50.3 | 23.6 | 20.9 | 7.63 | 8.1 | 12.1 | 9.95 | 5.4 | 11.3 |
| 0 | 35.2 | 12.4 | 5.55 | 4.78 | −4.78 | 2.47 | 1.62 | 3.47 | −5.71 |

TABLE 33
Antiproliferative Activity of Salmeterol (SAL) and Trequinsin against
Human Multiple Myeloma Cells (ANBL-6)

| | Trequinsin (μM) | | | | | | | | |
|---|---|---|---|---|---|---|---|---|---|
| SAL (μM) | 10.1 | 3.38 | 1.13 | 0.375 | 0.125 | 0.0417 | 0.0139 | 0.00464 | 0 |
| 0.0203 | 94.1 | 88 | 83.3 | 76.5 | 66.8 | 62.7 | 56.6 | 60.2 | 58 |
| 0.0101 | 93.3 | 88.4 | 80.5 | 71.3 | 62.6 | 61.1 | 56.2 | 59.3 | 54.7 |
| 0.00506 | 94.2 | 86.3 | 80.6 | 71.7 | 69 | 59.5 | 60.6 | 58 | 55 |
| 0.00253 | 93.2 | 87.8 | 81.3 | 74.4 | 65.7 | 62.7 | 59.3 | 61.3 | 54 |
| 0.00127 | 92 | 89.5 | 80.1 | 73 | 65.7 | 60.6 | 61.7 | 63.1 | 55.7 |
| 0.000633 | 93.6 | 88.4 | 81.3 | 73 | 69.4 | 64 | 58.2 | 57.1 | 58.4 |
| 0.000316 | 93.6 | 86.9 | 80.1 | 72.2 | 63.9 | 57.5 | 61.8 | 62.1 | 54.8 |
| 0.000158 | 92.7 | 89 | 81.6 | 71.3 | 65.5 | 56.4 | 55.1 | 53.4 | 50.7 |
| 0 | 50.3 | 28.6 | 17.4 | 7.82 | 4.39 | −4.22 | −1.74 | 2.91 | −3.34 |

BAR agonists also synergize with histone deacetylase (HDAC) inhibitors. The synergy scores for salmeterol in combination with the HDAC inhibitors MS-275, scriptaid, suberoylanilide hydroxamic acid (SAHA), and trichostatin A using the multiple myeloma cell line MM.1S are shown in Table 34. Representative combination data (salmeterol and SAHA) are provided in Table 35.

TABLE 34
Summary of Synergy Scores for the BAR Agonists Salmeterol
Combined with HDAC Inhibitors in the Multiple Myeloma
Cell Line MM.1S.

| Combination | Synergy score |
|---|---|
| salmeterol × MS-275 | 1.98 |
| salmeterol × scriptaid | 2.96 |
| salmeterol × SAHA | 2.01 |
| salmeterol × trichostatin A | 4.11 |

TABLE 35
Antiproliferative Activity of Salmeterol and SAHA Against
Human Multiple Myeloma Cells (MM.1S)

| | SAHA (μM) | | | | | |
|---|---|---|---|---|---|---|
| Salmeterol (nM) | 7.1 | 3.6 | 1.8 | 0.89 | 0.44 | 0 |
| 0.011 | 100 | 100 | 100 | 90 | 88 | 78 |
| 0.0036 | 100 | 100 | 99 | 92 | 79 | 70 |
| 0.0012 | 100 | 100 | 99 | 90 | 82 | 70 |
| 0.0004 | 100 | 100 | 99 | 87 | 78 | 65 |
| 0.00013 | 100 | 100 | 98 | 86 | 72 | 48 |
| 0.000045 | 100 | 100 | 96 | 72 | 48 | 22 |
| 0 | 100 | 100 | 83 | 34 | 25 | 0.7 |

BAR agonists also have synergistic activity when used in combination with HSP-90 inhibitors. Data for the combination of salmeterol and 17-AAG are shown in Table 36.

TABLE 36
Antiproliferative Activity of Salmeterol and 17-AAG Against
Human Multiple Myeloma Cells (MM.1S)

| | 17-AAG (μM) | | | | | |
|---|---|---|---|---|---|---|
| Salmeterol (nM) | 0.31 | 0.15 | 0.077 | 0.039 | 0.019 | 0 |
| 0.011 | 89 | 81 | 74 | 80 | 75 | 81 |
| 0.0036 | 82 | 73 | 72 | 72 | 69 | 64 |
| 0.0012 | 78 | 69 | 69 | 67 | 66 | 65 |
| 0.0004 | 74 | 61 | 63 | 60 | 61 | 60 |
| 0.00013 | 62 | 54 | 56 | 53 | 49 | 51 |
| 0.000045 | 47 | 35 | 36 | 38 | 43 | 19 |
| 0 | 17 | 12 | 3.1 | 1.7 | 3 | 1.1 |

### Example 3

### BAR Agonist Drug Combinations Potently Induce Apoptosis and Cell Death

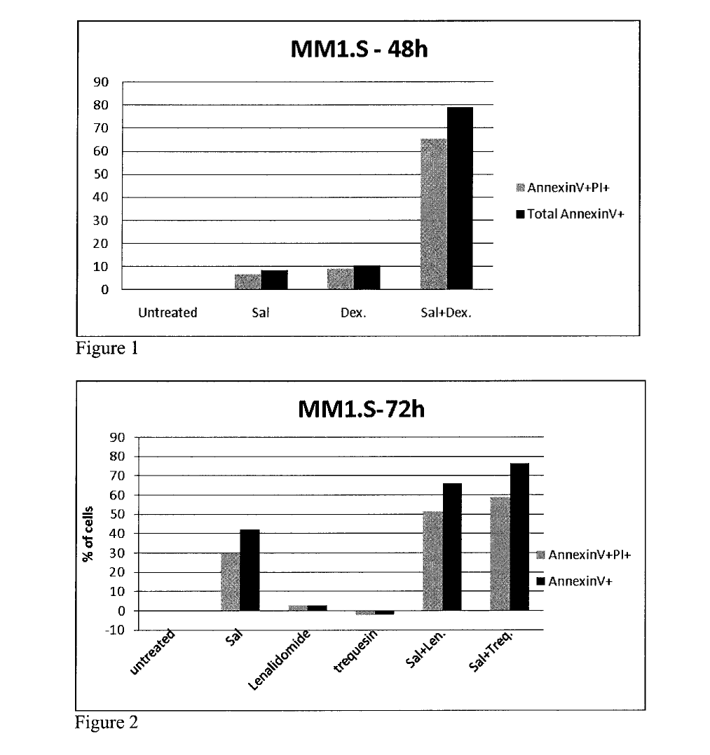

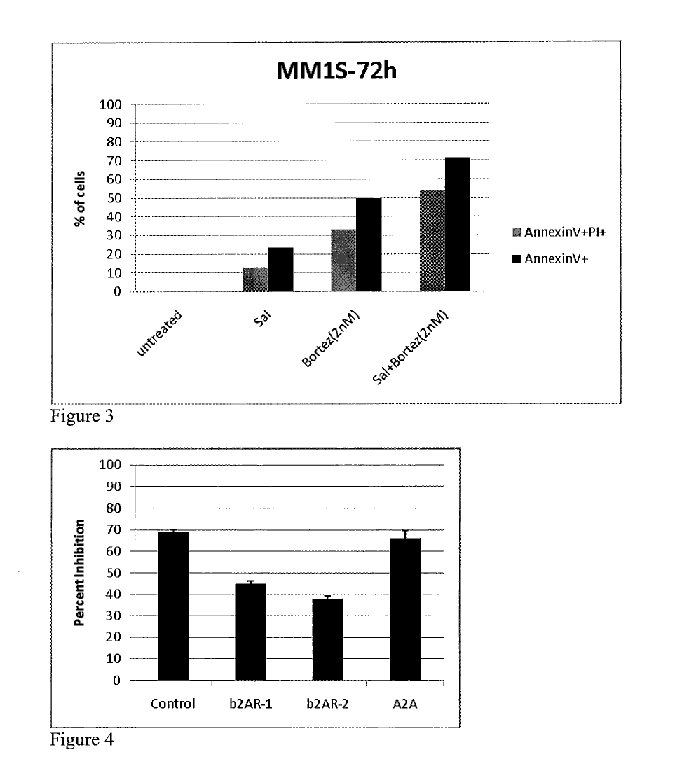

BAR agonists were highly synergistic and potently antiproliferative in combination with dexamethasone, melphalan, lenalidomide, and bortezomib as determined using an assay that measures ATP, a surrogate for the measurement of cell health and number. MM.1S cells were further treated with the BAR agonist salmeterol and either dexamethasone, lenalidomide, trequinsin, or bortezomib, as single agents or in combination with salmeterol. Effects on cell viability were determined by measuring the percent of cells that were annexin V positive (an early marker for apoptosis) and by measuring the percent of cells propidium iodide (PI) positive, an indicator that cellular membranes are compromised and a marker of cell death. FIG. 1 shows the results for MM.1S cells treated with 0.13 nM salmeterol and 20 nM dexamethasone for 48 hours as single agents or in combination. While neither agent had appreciable activity (<10%), use in combination resulted in ˜80% of the cells becoming annexin V positive. Combination-induction of apoptosis was also observed with salmeterol-lenalidomide and salmeterol-trequinsin (FIG. 2). Treatment of cells with 0.3 nM salmeterol resulted in ˜40% induction of MM.1S cell apoptosis after 72 hours. In contrast, 1 μM lenalidomide or 2 μM trequinsin at 72 hours had negligible effects. These drugs in combination synergistically induced apoptosis; salmeterol-lenalidomide treated cells were 65% annexin V positive, while salmeterol-trequinsin treated cells were 75% annexin V positive. Finally, the activity of salmeterol and bortezomib was determined (FIG. 3). MM.1S cells treated with 0.13 nM salmeterol for 72 hours were 22% annexin V positive, and cells cultured in the presence of 2 nM bortezomib were 50% annexin V positive. Use of these drugs in combination resulted in the cells being 71% annexin positive. In summary, BAR agonists used in combination with drugs used to treat multiple myeloma resulted in the rapid synergistic induction of apoptosis and cell death.

### Example 4

### BAR Agonist Drug Combinations Synergistically Inhibit Viability as Determined Using Long Term Growth Conditions (CFU Assays)

BAR agonists have potent synergistic antiproliferative activities in combination with dexamethasone, lenalidomide, melphalan, and bortezomib, drugs commonly used to treat B cell malignancies. BAR agonist combinations synergistically induce apoptosis of cells in culture. The effect of BAR agonists on cells grown in soft agar was also determined. This approach allows the measurement of long term cell viability after single agent or combination treatment. RPMI-8226 cells were treated with 2 nM salmeterol, 100 nM bortezomib, 200 nM bortezomib, or the combinations of both drugs for 5 hours. Cells were washed and plated in soft agar. After three weeks colonies were counted to determine cell viability. For each 10,000 cells plated, 740 colonies were observed if cells had not been exposed to drugs. Exposure of cells to 2 nM salmeterol reduced the colony number to 360, while exposing cells to 100 nM bortezomib reduced the colony forming number to 39. The combination of both drugs reduced the colony forming number to 8. Similar effects were observed when the bortezomib concentration was increased to 200 nM, where 7 colonies were observed with the number reduced to 3 by combination drug activity.

### Example 5

### Selectivity-Combination Activity in Normal Cells

With the observation that BAR agonists have strong synergistic antiproliferative activity across a large panel of representative B cell malignancy cell lines, combination activity was examined using non-transformed cells to question whether activity was selective. Strong combination activity with transformed cells but not normal counterparts suggests the potential for a “therapeutic window” where tumor cells are selectively killed over normal cells. Human coronary artery endothelial cells (HCAEC) were cultured in the presence of salmeterol and dexamethasone or salmeterol and lenalidomide for 72 hours using the conditions described for Table 10. The synergy scores were 0.031 for salmeterol and dexamethasone and 0.019 for salmeterol and lenalidomide. Similarly, the combination activity using PBMCs was 0.059 for Salmeterol and dexamethasone and 0.224 for salmeterol and lenalidomide. Combination activity was not observed with either drug combination using either HCAEC or PBMC.

### Example 6

### The Beta 2 Adrenergic Receptor is Important for Activity

BAR agonists employed in the above examples primarily target the beta 2 adrenergic receptor subtype. To determine whether a beta 1 adrenergic receptor agonist has similar activity, the beta 1 agonist dobutamine was combined with dexamethasone, and anti-proliferative activity determined using the multiple myeloma cell lines MM.1S and EJM. Dobutamine is a beta 1 agonist with 6 to 10-fold selectivity for beta 1 vs. beta 2 receptor (J Clin Invest 1981 67(6): 1703-11). The synergy score for dobutamine×dexamethasone for MM.1S was 2.61, and with EJM cells, a synergy score of 1.93 was observed. The 6×6 data for this combination are shown in Tables 41 and 42. For generation of the data in Tables 37 and 38, as with the 9×9 crosses, inhibition of proliferation was measured as described, after incubation of cells with test compound(s) for 72 hours. The effects of various concentrations of single agents or agents in combination were compared to control wells (cells not treated). The effects of agents alone and in combination are shown as percent inhibition of cell proliferation. As with beta 2 agonists, the beta 1 agonist dobutamine has potent combination activity when combined with dexamethasone.

TABLE 37
Antiproliferative Activity of Dexamethasone (DEX) and Dobutamine
Against Human Multiple Myeloma Cells (MM.1S)

| | Dobutamine (μM) | | | | | |
|---|---|---|---|---|---|---|
| DEX (μM) | 0.506 | 0.253 | 0.127 | 0.0633 | 0.0316 | 0 |
| 0.15 | 94.9 | 90.8 | 88.7 | 78.9 | 80.8 | 62.9 |
| 0.0502 | 86.6 | 86.8 | 78.8 | 68.7 | 67.5 | 50.5 |
| 0.0167 | 67 | 82.2 | 45 | 57.1 | 43.3 | 37.2 |
| 0.00557 | 59.2 | 42 | 40 | 14.2 | 33 | 2.23 |
| 0.00186 | 23.8 | 26.4 | 13 | 18.7 | 5.75 | 10.5 |
| 0 | 22.9 | −0.808 | 18.1 | −13.4 | −2.04 | 9.46 |

TABLE 38
Antiproliferative Activity of Dexamethasone (DEX) and
Dobutamine Against Human Multiple Myeloma Cells (EJM)

| | Dobutamine (μM) | | | | | |
|---|---|---|---|---|---|---|
| DEX (μM) | 0.506 | 0.253 | 0.127 | 0.0633 | 0.0316 | 0 |
| 1.02 | 69.9 | 63.2 | 51.2 | 38.7 | 24.5 | 0.767 |
| 0.341 | 51.5 | 40.2 | 38.4 | 17.6 | 5.92 | −11.2 |
| 0.114 | 52.5 | 43.4 | 24.5 | 18.7 | 6.34 | 4.81 |
| 0.0379 | 63.8 | 28.9 | 2.72 | 0.767 | 3.97 | −13.6 |
| 0.0126 | 35.1 | 29.8 | −1.05 | −2.86 | −9.27 | −7.6 |
| 0 | 21.5 | 3.14 | 3.55 | −4.11 | −2.3 | −9.13 |

To confirm that the activity of BAR agonists is beta adrenergic receptor-dependent, we examined the anti-proliferative activity of salmeterol, in the presence of increasing concentrations of the beta 1 and beta 2 adrenergic receptor antagonist CGP 12177A. As shown in Table 39, the lowest concentration of CGP 12177A tested (0.0019 μM) potently blocked salmeterol activity.

TABLE 39
Antiproliferative Activity of Salmeterol (SAL) on Human Multiple
Myeloma Cells (MM.1S) in the Presence of CGP 12177A.

| | Salmeterol (μM) | | | | | |
|---|---|---|---|---|---|---|
| CGP 12177A (μM) | 0.01 | 0.0034 | 0.0011 | 0.00037 | 0.00012 | 0 |
| 0.3 | 3.3 | 1.1 | 3.6 | 4.9 | 4.3 | 3.3 |
| 0.150 | 4.5 | −2.7 | −3.9 | 4.3 | −3.9 | −4.5 |
| 0.076 | 10 | 2.3 | 2.1 | 4.1 | 5.7 | 0.9 |
| 0.038 | 18 | 12 | 3.3 | 2.6 | 6.2 | −3.3 |
| 0.0019 | 16 | 4.2 | −0.3 | −0.77 | 2.7 | −0.3 |
| 0 | 49 | 50 | 49 | 42 | 26 | 6.2 |

We have tested 13 BAR agonists and found that each synergized with dexamethasone (Table 10). Furthermore, BAR agonists synergize with lenalidomide (Table 21), melphalan (Table 22), doxorubicin (Table 23), bortezomib (Tables 28 and 29), A2A agonists and PDE inhibitors (Table 30), and HDAC inhibitors (Tables 34 and 35). All of the drugs assayed in these examples agonize the beta 2 adrenergic receptor but can be less selective at higher concentrations, activating other beta adrenergic receptor family members or possibly other cellular targets. To determine if the beta 2 receptor is necessary for the antiproliferative effects observed with B cells, we examined combination activity when BAR agonist was assayed in the presence of the highly selective beta 2 specific antagonist ICI 118,551.

Table 40 shows the potent synergy observed for the combination dexamethasone and clenbuterol in a 6×6 dose matrix format. The addition of 0.91 nM ICI 118,551 significantly reduced clenbuterol single agent activity (Table 41), while 9.2 nM ICI 118,551 (Table 42) completely blocked clenbuterol single agent activity and most of the synergy.

TABLE 40
Antiproliferative Activity of Clenbuterol and Dexamethasone
on Human Multiple Myeloma Cells (MM.1S).

| | Clenbuterol (μM) | | | | | |
|---|---|---|---|---|---|---|
| Dexamethasone (μM) | 0.01 | 0.0051 | 0.0025 | 0.0013 | 0.0006 | 0 |
| 0.15 | 100 | 100 | 100 | 100 | 100 | 70 |
| 0.076 | 100 | 100 | 99 | 99 | 100 | 63 |
| 0.038 | 99 | 99 | 98 | 99 | 99 | 56 |
| 0.019 | 99 | 99 | 99 | 98 | 100 | 47 |
| 0.0095 | 99 | 98 | 96 | 95 | 98 | 38 |
| 0 | 78 | 61 | 62 | 57 | 66 | 13 |

TABLE 41
Antiproliferative Activity of Clenbuterol and Dexamethasone
on Human Multiple Myeloma Cells (MM.1S) in the Presence of
0.91 μM ICI, 118,551

| Dexamethasone | Clenbuterol (μM) | | | | | |
|---|---|---|---|---|---|---|
| (μM) | 0.01 | 0.0051 | 0.0025 | 0.0013 | 0.0006 | 0 |
| 0.15 | 99 | 97 | 96 | 95 | 98 | 72 |
| 0.076 | 100 | 96 | 94 | 90 | 97 | 61 |
| 0.038 | 98 | 94 | 90 | 86 | 92 | 56 |
| 0.019 | 98 | 88 | 88 | 83 | 92 | 46 |
| 0.0095 | 94 | 82 | 74 | 67 | 84 | 34 |
| 0 | 54 | 15 | 21 | 7.8 | 28 | 1.5 |

TABLE 42
Antiproliferative Activity of Clenbuterol and Dexamethasone
on Human Multiple Myeloma Cells (MM.1S) in the Presence of
9.2 μM ICI, 118,551

| Dexamethasone | Clenbuterol (μM) | | | | | |
|---|---|---|---|---|---|---|
| (μM) | 0.01 | 0.0051 | 0.0025 | 0.0013 | 0.0006 | 0 |
| 0.15 | 93 | 84 | 84 | 78 | 86 | 71 |
| 0.076 | 92 | 77 | 74 | 68 | 78 | 60 |
| 0.038 | 83 | 62 | 58 | 56 | 66 | 56 |
| 0.019 | 79 | 50 | 52 | 47 | 56 | 48 |
| 0.0095 | 86 | 78 | 66 | 56 | 41 | 31 |
| 0 | 5.2 | −5.1 | −7.1 | 4.7 | 6.8 | 3.6 |

When dexamethasone and dobutamine were combined, some synergy was observed but only at high concentrations of dobutamine (Table 43). The addition of 0.91 nM ICI 118,551, dramatically reduced combination activity, with residual synergy observed only at the highest concentration of dobutamine tested (Table 44).

TABLE 43
Antiproliferative Activity of Dobutamine and Dexamethasone
on Human Multiple Myeloma Cells (MM.1S)

| | Dobutamine (μM) | | | | | |
|---|---|---|---|---|---|---|
| Dexamethasone (μM) | 1.0 | 0.51 | 0.25 | 0.13 | 0.063 | 0 |
| 0.15 | 98 | 87 | 87 | 86 | 81 | 72 |
| 0.076 | 96 | 86 | 81 | 71 | 70 | 63 |
| 0.038 | 94 | 80 | 72 | 66 | 68 | 60 |
| 0.019 | 91 | 70 | 66 | 61 | 63 | 47 |
| 0.0095 | 83 | 60 | 55 | 41 | 47 | 33 |
| 0 | 28 | 12 | 4.7 | 14 | 11 | 8.7 |

TABLE 44
Antiproliferative Activity of Dobutamine and Dexamethasone
on Human Multiple Myeloma Cells (MM.1S) in the Presence of
0.91 μM ICI, 118,551

| | Dobutamine (μM) | | | | | |
|---|---|---|---|---|---|---|
| Dexamethasone (μM) | 1.0 | 0.51 | 0.25 | 0.13 | 0.063 | 0 |
| 0.15 | 89 | 79 | 86 | 76 | 75 | 71 |
| 0.076 | 79 | 70 | 66 | 63 | 62 | 63 |
| 0.038 | 73 | 52 | 54 | 54 | 55 | 54 |
| 0.019 | 62 | 44 | 45 | 44 | 49 | 43 |
| 0.0095 | 47 | 31 | 30 | 31 | 35 | 32 |
| 0 | −1.8 | −2.8 | −5.4 | 2 | 2.2 | 1.9 |

The beta 3 agonist BRL37344 was not very active as a single agent, but some combination synergy was observed with dexamethasone when high concentrations of BRL37344 were used (20 nM, Table 45). BRL37344 is a beta 3 agonist that is 76-fold selective for beta 3 vs. beta 2 (Mol Phar 1994 46(2):357-63). The combination activity of BRL37344 and dexamethasone was inhibited in the presence of 0.91 nM of the beta 2 antagonist ICI 118,551 (Table 46).

TABLE 45
Antiproliferative Activity of BRL 37344 and Dexamethasone
on Human Multiple Myeloma Cells (MM.1S)

| | BRL 37344 (μM) | | | | | |
|---|---|---|---|---|---|---|
| Dexamethasone (μM) | 0.02 | 0.01 | 0.0051 | 0.0025 | 0.0013 | 0 |
| 0.15 | 94 | 84 | 81 | 78 | 75 | 69 |
| 0.076 | 88 | 76 | 74 | 71 | 68 | 62 |
| 0.038 | 84 | 65 | 72 | 64 | 52 | 58 |
| 0.019 | 74 | 63 | 58 | 50 | 50 | 49 |
| 0.0095 | 70 | 47 | 44 | 38 | 44 | 33 |
| 0 | 10 | 10 | 8.3 | 7.7 | 4 | 3.9 |

TABLE 46
Antiproliferative Activity of BRL 37344 and Dexamethasone
on Human Multiple Myeloma Cells (MM.1S) in the Presence of
0.92 μM ICI 118,551

| | BRL 37344 (μM) | | | | | |
|---|---|---|---|---|---|---|
| Dexamethasone (μM) | 0.02 | 0.01 | 0.0051 | 0.0025 | 0.0013 | 0 |
| 0.15 | 79 | 77 | 73 | 74 | 73 | 68 |
| 0.076 | 67 | 64 | 64 | 64 | 60 | 60 |
| 0.038 | 56 | 47 | 55 | 52 | 50 | 53 |
| 0.019 | 45 | 40 | 44 | 42 | 47 | 42 |
| 0.0095 | 28 | 32 | 27 | 33 | 37 | 32 |
| 0 | −12 | −8.2 | −4.9 | 3.2 | 0.2 | −0.3 |

The results obtained with the beta receptor antagonist ICI 118,551 point to the beta 2 receptor subtype as being important for the antiproliferative effect of agonists on cell growth. We also examined the antiproliferative activity of BAR agonists when the MM cell line MM.1R was transfected with siRNA targeting the beta 2 receptor or two control siRNA, one (control) designed using scrambled sequences so that cellular transcripts are not targeted and a second siRNA (A2A) that reduces expression of the A2A receptor RNA by 75% as determined by PCR analysis. Specific gene silencing was greater than 60% as determined by real time PCR analysis 48 hours post-transfection. At 48 hours post-transfection, cells were exposed to the beta 2 receptor agonist salbutamol and incubated an additional 72 hours, and the compounds were assayed for antiproliferative activity. Representative data are shown in FIG. 4. Cells transfected with A2A receptor siRNA or a control siRNA had similar sensitivity to salbutamol (65-68% inhibition of proliferation). In contrast, two different siRNA that targeted the beta 2 receptor reduced salbutamol antiproliferative activity to 39-45%.

### Example 7

### Tumor Cell Resistance Profiles after Chronic Exposure to Beta Adrenergic Agonists

A recurrent problem in cancer therapy is that prolonged exposure of tumor cells to chemotherapeutic agents can generate cells resistant to agents that initially have antiproliferative or cell killing activity. To determine if multiple myeloma cells become resistant to the effects of BAR agonists after prolonged exposure, MM.1S cells were cultured in the presence of increasing concentrations of either salmeterol or salbutamol over a one month period such that at the end of 30 days, the concentration of drug was 64 nM for salmeterol and 150 nM for salbutamol. Cells cultured for one month in the presence of BAR agonist were washed to remove drug and put into 384 well plates for 9×9 dose matrix combination screening with BAR agonists and dexamethasone. As a control, combination screening was performed using MM.1S cells that did not have prior exposure to BAR agonists. Table 47 shows a 9×9 combination matrix for salmeterol and dexamethasone in naïve cells, the result being very similar to the data shown in Table 17 with the combination having potent synergistic antiproliferative activity. After culture in 64 nM salmeterol, cells were no longer sensitive to salmeterol when deployed as a single agent; however, some combination synergistic activity was still observed (Table 48). For example, the combination of 0.0022 μM salmeterol (4.1% inhibition) and 0.0082 μM dexamethasone (51% inhibition) inhibited proliferation by 70% when used in combination. Chronic exposure of cells to salmeterol had a striking effect on dexamethasone single agent activity. While dexamethasone inhibited the proliferation of naïve cells ˜70% at 2.0-0.23 μM (Table 47), effects on proliferation were more pronounced for cells exposed long term to salmeterol with ˜96% inhibition observed at 2.0-0.23 μM dexamethasone (Table 48). This boost in dexamethasone single agent activity was not observed with cells cultured for one month in the presence of salbutamol (compare Tables 49 and 50), but salbutamol/dexamethasone synergy was still observed (Table 50).

The dose matrix results for MM.1S cells cultured in the presence of BAR agonists for one month show that the cells were insensitive to BAR agonists when used as a single agent. However, synergistic antiproliferative activity was still observed in combination with dexamethasone. Similar results were observed for BAR agonists in combination with melphalan. The BAR agonists were no longer active as single agents but synergized with melphalan (Table 51). However, unlike with dexamethasone, melphalan single agent activity was not potentiated.

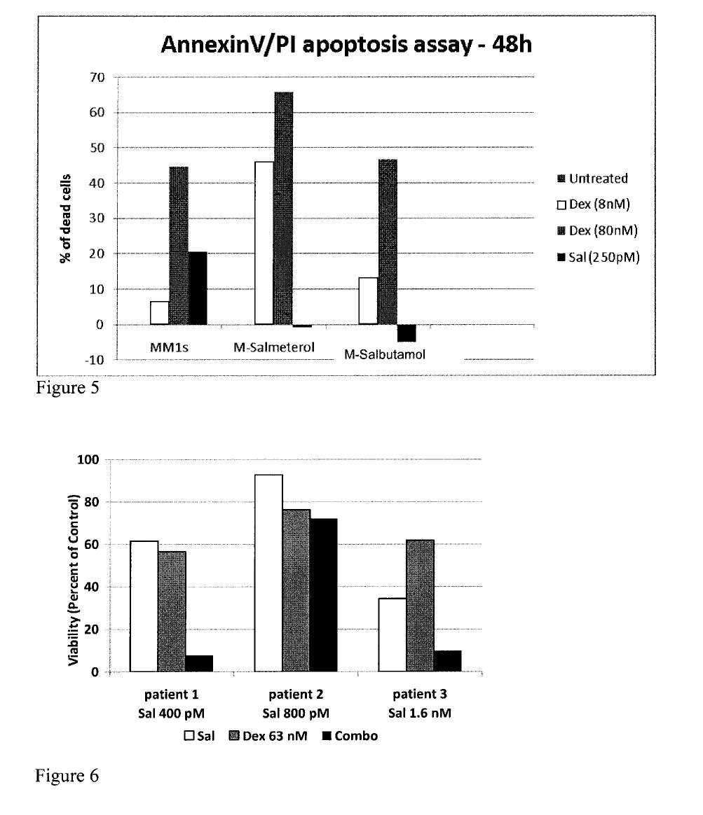

The observation that cells treated for one month with salmeterol were hypersensitive to dexamethasone (compare dexamethasone single agent activity for Table 47 and 48) has interesting clinical ramifications. As an independent approach to confirm that tumor cells become hypersensitive to salmeterol, MM.1S cells treated with salmeterol or salbutamol for one month were incubated with either low (8 nM) or high (80 nM) dexamethasone or 250 pM salmeterol. Forty eight hours later, the percent of cells positive for Annexin V and propidium iodide (PI) was determined using FACS (FIG. 5). Control cells not exposed to BAR agonist for one month were sensitive to salmeterol (20% Annexin V/PI+) and both low and high concentrations of dexamethasone (8 and 45% Annexin V/PI+). As expected, cells treated for one month with salmeterol or salbutamol were not sensitive to 250 pM salmeterol. However, cells exposed to salmeterol for one month were hypersensitive to dexamethasone, with 47% Annexin V/PI+ cells found with 8 nM dexamethasone treatment while 80 nM dexamethasone treatment resulted in 65% of the cells becoming Annexin V/PI+.

TABLE 47
Antiproliferative Activity of Salmeterol and Dexamethasone on
Human Multiple Myeloma Cells (MM.1S)

| | Salmeterol | | | | | | | | |
|---|---|---|---|---|---|---|---|---|---|
| | (μM) | | | | | | | | |
| DEX (μM) | 0.02 | 0.0066 | 0.0022 | 0.00074 | 0.00024 | 0.00008 | 0.000027 | 0.0000091 | 0 |
| 2.0 | 96 | 96 | 96 | 96 | 95 | 96 | 92 | 86 | 70 |
| 0.666 | 96 | 96 | 96 | 96 | 96 | 95 | 90 | 83 | 71 |
| 0.23 | 96 | 95 | 96 | 96 | 96 | 95 | 90 | 80 | 68 |
| 0.074 | 96 | 96 | 96 | 96 | 96 | 94 | 88 | 76 | 59 |
| 0.024 | 96 | 94 | 96 | 94 | 95 | 90 | 82 | 67 | 48 |
| 0.0082 | 94 | 94 | 94 | 92 | 90 | 79 | 70 | 48 | 39 |
| 0.00274 | 86 | 87 | 87 | 82 | 80 | 53 | 38 | 25 | 15 |
| 0.0009 | 72 | 76 | 71 | 67 | 62 | 33 | 31 | 14 | 11 |
| 0 | 59 | 53 | 56 | 51 | 44 | 25 | 7.1 | 4.9 | 3.4 |

TABLE 48
Antiproliferative Activity of Salmeterol and Dexamethasone on
Human Multiple Myeloma Cells (MM.1S) Cultured in the Presence
of Salmeterol for One Month (final concentration 64 nM)

| | Salmeterol | | | | | | | | |
|---|---|---|---|---|---|---|---|---|---|
| | (μM) | | | | | | | | |
| | (1 month 64 nM Salmeterol) | | | | | | | | |
| DEX (μM) | 0.02 | 0.0066 | 0.0022 | 0.00074 | 0.00024 | 0.00008 | 0.000027 | 0.0000091 | 0 |
| 2.0 | 97 | 96 | 96 | 97 | 96 | 96 | 96 | 96 | 96 |
| 0.666 | 96 | 96 | 97 | 97 | 96 | 95 | 95 | 96 | 96 |
| 0.23 | 97 | 97 | 96 | 96 | 96 | 96 | 96 | 95 | 96 |
| 0.074 | 96 | 95 | 96 | 96 | 96 | 95 | 95 | 94 | 93 |
| 0.024 | 91 | 91 | 92 | 90 | 93 | 83 | 88 | 87 | 81 |
| 0.0082 | 67 | 73 | 70 | 64 | 71 | 55 | 65 | 58 | 51 |
| 0.00274 | 38 | 41 | 36 | 36 | 41 | 23 | 38 | 37 | 23 |
| 0.0009 | 21 | 19 | 17 | 17 | 18 | 13 | 13 | 7.5 | 8.2 |
| 0 | 5.6 | 3.6 | 4.1 | 9.5 | 6.4 | 6.1 | 2 | 6.7 | 2.6 |

TABLE 49
Antiproliferative Activity of Salbutamol and Dexamethasone on
Human Multiple Myeloma Cells (MM.1S)

| | Salbutamol | | | | | | | | |
|---|---|---|---|---|---|---|---|---|---|
| | (μM) | | | | | | | | |
| DEX (μM) | 0.2 | 0.1 | 0.05 | 0.025 | 0.0125 | 0.00625 | 0.00375 | 0.0000091 | 0 |
| 2.0 | 96 | 96 | 96 | 96 | 96 | 94 | 91 | 92 | 75 |
| 0.666 | 96 | 96 | 96 | 96 | 95 | 95 | 92 | 90 | 75 |
| 0.23 | 96 | 96 | 95 | 96 | 95 | 93 | 93 | 90 | 73 |
| 0.074 | 96 | 96 | 96 | 95 | 96 | 94 | 91 | 87 | 63 |
| 0.024 | 95 | 96 | 94 | 95 | 93 | 88 | 82 | 74 | 47 |
| 0.0082 | 92 | 92 | 90 | 87 | 84 | 74 | 72 | 67 | 37 |
| 0.00274 | 87 | 82 | 80 | 72 | 72 | 51 | 43 | 39 | 12 |
| 0.0009 | 76 | 70 | 66 | 56 | 53 | 34 | 24 | 22 | 13 |
| 0 | 58 | 55 | 51 | 38 | 39 | 30 | 15 | 10 | 2.4 |

TABLE 50
Antiproliferative Activity of Salbutamol and Dexamethasone on
Human Multiple Myeloma Cells (MM.1S) Cultured in the Presence
of Salbutamol for One Month (final concentration 150 nM)

| | Salbutamol | | | | | | | | |
|---|---|---|---|---|---|---|---|---|---|
| | (μM) | | | | | | | | |
| | (1 month 150 nM Salbutamol) | | | | | | | | |
| DEX (μM) | 0.2 | 0.1 | 0.05 | 0.025 | 0.0125 | 0.00625 | 0.00375 | 0.0000091 | 0 |
| 2.0 | 95 | 93 | 93 | 92 | 89 | 80 | 72 | 73 | 61 |
| 0.666 | 94 | 93 | 92 | 92 | 90 | 84 | 73 | 72 | 60 |
| 0.23 | 94 | 93 | 91 | 90 | 89 | 78 | 70 | 71 | 59 |
| 0.074 | 92 | 90 | 90 | 87 | 84 | 77 | 71 | 60 | 48 |
| 0.024 | 84 | 79 | 80 | 78 | 67 | 57 | 57 | 51 | 39 |
| 0.0082 | 62 | 66 | 60 | 47 | 56 | 37 | 39 | 34 | 30 |
| 0.00274 | 41 | 36 | 31 | 26 | 24 | 17 | 21 | 17 | 10 |
| 0.0009 | 16 | 8.8 | 14 | 8.3 | 14 | 8.8 | 2.9 | 7.5 | 6.5 |
| 0 | 6.8 | 11 | 1.5 | 4.6 | −1.9 | 4.6 | −5.8 | −5.3 | −7.8 |

TABLE 51
Summary of Synergy Scores for the BAR agonists Salmeterol and
Salbutamol Combined with Dexamethasone or Melphalan in Naïve
MM.1S Cells or Cells Cultured in the Presence of Salmeterol or
Salbutamol for One Month

| | | MM.1S/1 month | MM.1S 1 month |
|---|---|---|---|
| | | Salbutamol | Salmeterol |
| Combination | MM.1S | (150 nM) | (64 nM) |
| salmeterol × | 18.05 | 13.27 | 2.26 |
| dexamethasone | | | |
| salbutamol × | 10.66 | 8.26 | 1.61 |
| dexamethasone | | | |
| salmeterol × | 4.17 | 3.5 | 2.02 |
| melphalan | | | |
| salbutamol × | 2.94 | 2.31 | 1.75 |
| melphalan | | | |

### Example 8

### Combinations with IL-6

Myeloma cells migrate to bone, where they form tumors called plasmocytomas. These malignant cells express cell adhesion molecules that allow attachment and communication with cells of the bone marrow microenvironment. This communication influences myeloma cell survival and growth as MM cells secrete a number of cytokines that act on bone marrow stromal cells (BMSCs), which, in turn, secrete factors that contribute to the growth and proliferation of MM cells. One factor, IL-6, is a central regulatory cytokine in the pathogenesis of MM. This cytokine causes proliferation of MM cells and inhibits cancer drug sensitivity/apoptosis.

The protective effect of IL-6 can be observed with MM cells in culture. Other investigators have shown that MM.1S cell proliferation is stimulated by IL-6, and the cells are more resistant to chemotherapeutic agents such as dexamethasone and rapamycin. Shown in Tables 52-53 is the 6×6 analysis of salmeterol×dexamethasone (in triplicate) using MM.1S cells −/+IL-6. Consistent with what has been described by others, the proliferation of MM.1S cells is inhibited 52% when cultured in the presence of 0.15 μM dexamethasone. In contrast, when 10 ng/ml IL-6 is present in the media, the inhibitory effect of dexamethasone is approximately halved, with MM.1S proliferation inhibited only 24%. In contrast, IL-6 increased BAR agonist activity. The antiproliferative activity of salmeterol was increased at each concentration tested (compare Tables 52 and 53). Also, shown (Table 54) is an 8-point titration of salmeterol using MM.1S cells (in quadruplicate) cultured in the presence or absence of 10 ng/ml IL-6. Again, IL-6 increases the activity of the BAR agonist salmeterol. BAR agonists are a class of drugs that should be more active in the bone tumor microenvironment, having a more potent anti-tumor effect due to the presence of IL-6.

TABLE 52
Antiproliferative Activity of Dexamethasone (DEX) and
Salmeterol Against Human Multiple Myeloma Cells (MM.1S)
without IL-6.

| | Salmeterol (μM) | | | | | |
|---|---|---|---|---|---|---|
| | (no IL-6) | | | | | |
| DEX (μM) | 0.00051 | 0.000253 | 0.00013 | 0.0000633 | 0.000032 | 0 |
| 0.15 | 93 | 93 | 91 | 89 | 83 | 52 |
| 0.076 | 94 | 92 | 87 | 88 | 86 | 45 |
| 0.038 | 92 | 91 | 88 | 85 | 73 | 44 |
| 0.019 | 90 | 88 | 89 | 75 | 77 | 32 |
| 0.0096 | 84 | 81 | 77 | 73 | 66 | 27 |
| 0 | 46 | 48 | 38 | 21 | 21 | 7.1 |

TABLE 53
Antiproliferative Activity of Dexamethasone (DEX) and
Salmeterol Against Human Multiple Myeloma Cells (MM.1S),
10 ng/ml IL-6

| | Salmeterol (μM) | | | | | |
|---|---|---|---|---|---|---|
| | (+10 ng/ml IL-6) | | | | | |
| DEX (μM) | 0.00051 | 0.000253 | 0.00013 | 0.0000633 | 0.000032 | 0 |
| 0.15 | 90 | 88 | 86 | 80 | 72 | 24 |
| 0.076 | 88 | 86 | 80 | 75 | 69 | 26 |
| 0.038 | 85 | 82 | 79 | 70 | 59 | 26 |
| 0.019 | 82 | 76 | 72 | 65 | 65 | 14 |
| 0.0096 | 76 | 71 | 64 | 60 | 54 | 12 |
| 0 | 52 | 51 | 41 | 29 | 22 | 3.6 |

TABLE 54
Antiproliferative Activity of Salmeterol Against Human Multiple Myeloma Cells
(MM.1S), 8 point titration −/+10 ng/ml IL-6

| Condition | 0.025 pM | 0.051 pM | 0.1 pM | 0.2 pM | 0.4 pM | 0.81 pM | 160 pM | 320 pM |
|---|---|---|---|---|---|---|---|---|
| salmeterol | 5 | 6.7 | 11 | 25 | 30 | 43 | 50 | 59 |
| (no IL-6) | | | | | | | | |
| salmeterol | 2.8 | 8.6 | 19 | 30 | 36 | 54 | 59 | 66 |
| (10 ng/mL | | | | | | | | |
| IL-6) | | | | | | | | |

### Example 9

### BAR Agonist Activity Using Multiple Myeloma Patient Tumor Cells

The observation that beta agonists synergize with drugs used for the treatment of multiple myeloma in multiple myeloma cell lines suggest that such a drug pairing may have value in the clinic for treatment of the disease. To address this possibility more directly, in addition to the analysis of cell lines, we examined that activity of BAR agonist (salmeterol) and dexamethasone using multiple myeloma tumor cells from patients. Tumor cells were obtained via bone marrow aspirates and affinity selected using CD138-linked magnetic beads. Purified tumor cells were incubated with salmeterol and dexamethasone using an 8×8 dose matrix format and 48 hours later, cell viability was determined using an MTT colorimetric assay. As shown in Table 55, the combination of salmeterol and dexamethasone exhibited potent synergistic activity. For example, 0.4 nM salmeterol had 38% activity, 0.063 μM dexamethasone 44% activity and the combination 92% activity.

TABLE 55
Reduction in Cell Viability of Dexamethasone (DEX) and Salmeterol
Against Tumor Cells from a Patient with Multiple Myeloma

| | Salmeterol | | | | | | | |
|---|---|---|---|---|---|---|---|---|
| | (nM) | | | | | | | |
| DEX (μM) | 0.8 | 0.4 | 0.2 | 0.1 | 0.05 | 0.025 | 0.0125 | 0 |
| 4.0 | 91 | 92 | 90 | 88 | 80 | 75 | 64 | 46 |
| 2.0 | 91 | 92 | 88 | 90 | 81 | 72 | 68 | 47 |
| 1.0 | 92 | 91 | 92 | 88 | 84 | 78 | 70 | 50 |
| 0.5 | 91 | 92 | 89 | 88 | 82 | 78 | 71 | 56 |
| 0.25 | 91 | 92 | 91 | 86 | 80 | 76 | 68 | 50 |
| 0.125 | 91 | 93 | 89 | 87 | 82 | 74 | 68 | 53 |
| 0.0625 | 91 | 92 | 91 | 87 | 78 | 72 | 61 | 44 |
| 0 | 44 | 38 | 40 | 35 | 13 | 12 | −0.2 | 0 |

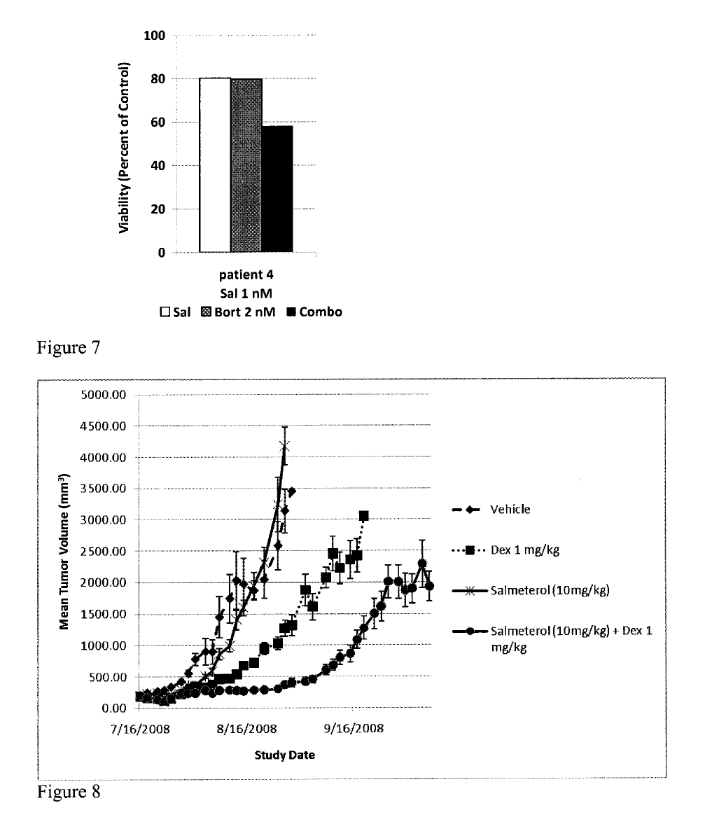

FIG. 6 shows single agent/combination data from Table 55 (patient 1) plus the results obtained using cells for two additional patients. Synergistic salmeterol/dexamethasone combination activity was clearly observed for patients 1 and 3. We have also examined the activity of salmeterol and bortezomib using patient tumor cells (FIG. 7). While tumor cell viability was 80% after treatment with either 2 nM bortezomib or 1 nM salmeterol for 48 hours, the drugs in combination reduced viability to 58%.

### Example 10

### Combination Activity In Vivo (Mouse Model)

1. BAR Agonist in Combination with Dexamethasone (MM.1S Cells)

To further characterize combination activity, we examined the ability of salmeterol in combination with the standard of care, dexamethasone (Dex), to inhibit the growth of human multiple myeloma xenografts and prolong survival in a severe combined immune deficient (SCID) mouse model as compared to each of the single agent components (FIG. 8).

The human multiple myeloma cell line MM1S was injected subcutaneously into the flanks of SCID mice. Five days following the injection of MM1S cells, animals were randomized into the following four treatment groups (n=6-7) based on tumor size.

### Group 1—Vehicle (90% PBS+10% EtOH)

Group 2—Dex (1 mg/kg)
Group 3—Salmeterol (10 mg/kg)
Group 4—Salmeterol (10 mg/kg)+Dex (1 mg/kg)

The prepared test, positive, and negative control substances were administered via subcutaneous injection daily for up to 85 Days. During the treatment period, tumor volume and animal body weights were measured 3 times per week (Monday, Wednesday and Friday). The animals were removed from the study if there was overall poor body condition, tumor volume above 3000 mm³, body weight loss greater than or equal to 20% or ulcerated tumors.

All of the animals in the Dex (1 mg/kg) study group were removed by the 66^{th} study day. All of the animals in the salmeterol (10 mg/kg) study group were removed by the 43^{rd} study day. Five of 7 animals in study group salmeterol (10 mg/kg)+Dex (1 mg/kg) survived until the final study day (85). The salmeterol (10 mg/kg)+Dex (1 mg/kg) study group had significantly greater survival when compared to the Dex (1 mg/kg) study group and the salmeterol (10 mg/kg) study group (p<0.05, ANOVA).

The percent change in tumor volume was calculated for the groups Dex (1 mg/kg), salmeterol (10 mg/kg), and salmeterol (10 mg/kg)+Dex (1 mg/kg) from study date Day 1 to Day 41. Day 41 was the last day that there was not more than one animal removed from each group. Results show that the percent change in tumor volume for each combination group was less than that of the single agent components.

The slope of the tumor volume growth of each animal was calculated from the initial study day until the day each animal was removed from the study. From these slopes the mean change in tumor volume per study day was calculated. The lowest mean change was calculated in the Salmeterol (10 mg/kg)+Dex (1 mg/kg) study group (18.36 mm³/day). No statistical significance was calculated between these two study groups and the single agent components.

Body weight gain was observed in all study groups. Study animals gained between 1.5 and 28.5 percent over the course of the study.

The combination of Salmeterol (10 mg/kg)+Dex (1 mg/kg) significantly improved survival, and reduced the percent change in tumor volume from Day 1 to Day 41 when compared to its single agent components. It also had the lowest mean change in tumor volume per study day compared with all other groups within the study.

2. BAR Agonist in Combination with Dexamethasone or Bortezomib (RPMI 8226 cells)

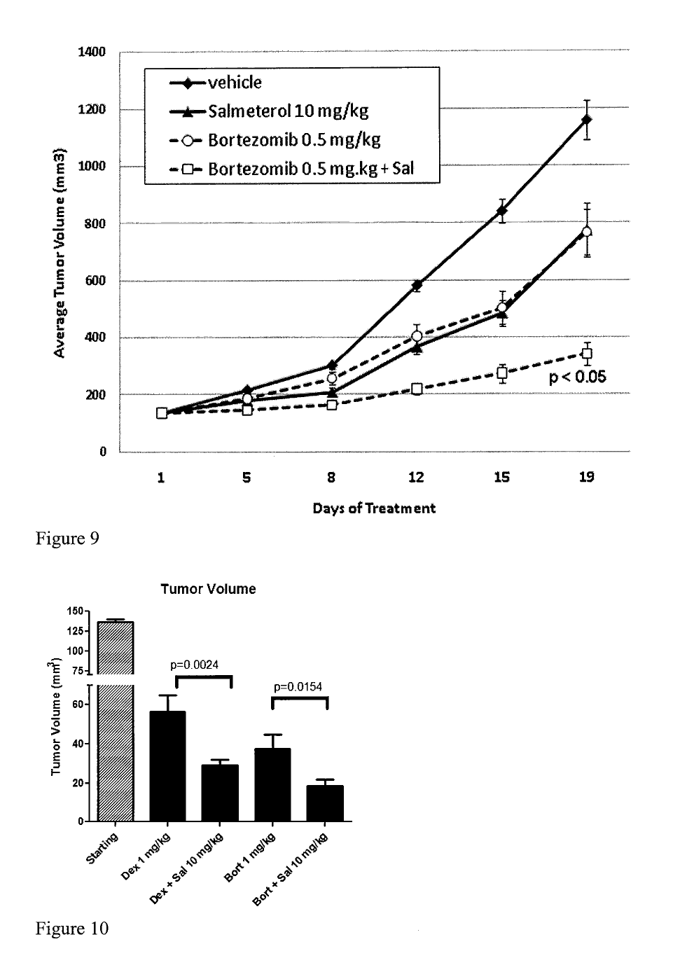

The anticancer activity of the beta adrenergic agonist salmeterol was also evaluated in the RPMI-8226 multiple myeloma (MM) xenograft model (FIG. 9). Salmeterol was administered alone and in combination with the standard of care (SOC) agents bortezomib and dexamethasone. Bortezomib was given at its MTD (1 mg/kg) and ½ its MTD (0.5 mg/kg). Dexamethasone was administered by intraperitoneal (IP) injection daily for thirty-six treatments (QD×36). Bortezomib was injected intravenously (IV) every third day for six treatments (Q3D×6).

### Group 1—Vehicle (90% PBS+10% EtOH)

Group 2—Dex (1 mg/kg)
Group 3—Bortezomib (0.5 mg/kg)
Group 4—Bortezomib (1 mg/kg)
Group 5—Salmeterol (10 mg/kg)
Group 6—Salmeterol (10 mg/kg)+Dex (1 mg/kg)
Group 7—Salmeterol (10 mg/kg)+Bortezomib (0.5 mg/kg)
Group 8—Salmeterol (10 mg/kg)+Bortezomib (0.5 mg/kg)

Significant endpoints included mean tumor growth inhibition (TGI) or regression, animal weight loss, and potential toxicity. The RPMI-8226 MM cell line was obtained from ATCC and cultured in media supplemented with 10% serum. Animals were implanted with cancer cells harvested from tissue culture and allowed to establish tumors in SCID mice. Treatment initiated when the mean tumor volume reached 137 mm³ in size.

Some initial weight loss was observed following treatment with 1 mg/kg bortezomib alone and in combination with salmeterol. However, animal weight in these two groups was regained at the completion of the study. Notably, salmeterol was well-tolerated and did not significantly alter the weights of the animals compared to the vehicle group. The dexamethasone and 0.5 mg/kg bortezomib groups also did not induce much weight loss. Importantly, no animal deaths occurred in any group throughout the duration of the study. Both 1 mg/kg bortezomib and dexamethasone treatments dramatically inhibited tumor growth (TGIs of >100%) in this study compared to animals that were administered the vehicle. 10 mg/kg salmeterol and 0.5 mg/kg bortezomib both exhibited moderate antitumor activity and produced TGIs of 37% and 38% respectively on Day 19 of the study. The combination of 0.5 mg/kg bortezomib and salmeterol resulted in enhanced antitumor activity yielding a TGI of 80% on Day 19. Combinations of salmeterol with 1 mg/kg bortezomib and 1 mg/kg dexamethasone were also more effective at reducing tumor burden than either single agent, causing regression of tumor size (FIG. 10).

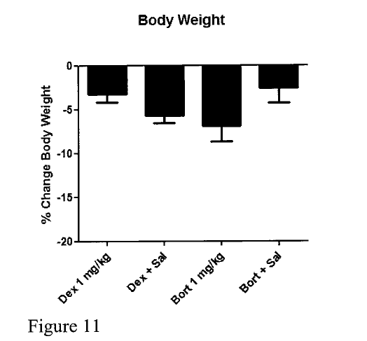

Overall, Salmeterol was well-tolerated and demonstrated some anticancer activity in the RPMI-8226 MM xenograft model. Importantly, salmeterol enhanced the anticancer activity of both dexamethasone and bortezomib without adding any additional toxicity (weight loss, FIG. 11). This effect was most pronounced when combined with bortezomib given at ½ its MTD. Since no toxicity was observed in terms of animal weight loss or death, an increase in the dose of salmeterol may further increase the drug's efficacy. Taken together, salmeterol possessed anticancer activity as a single agent and appears to enhance the activity of SOC agents. Salmeterol warrants further investigation, especially in combination with SOC agents, for the treatment of MM and potentially other malignancies.

### Other Embodiments

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

What is claimed is:

1. (canceled)

2. (canceled)

3. (canceled)

4. A method of treating a B-cell proliferative disorder, said method comprising administering to a patient a combination of a BAR agonist and a second compound selected from a PDE inhibitor, an A2A receptor agonist, an antiproliferative compound, and an IL-6 agonist, in an amount effective to treat said B-cell proliferative disorder.

5. (canceled)

6. The method of claim 4, wherein said BAR agonist is selected from the group consisting of arbutaline, arfomoterol, bambuterol, bitolterol, broxaterol, clenbuterol, fenoterol, formoterol, hexoprenaline, indacaterol, isoetharine, isoproterenol, levalbuterol, meluadrine, metaproterenol, nylidrin, picumeterol, pirbuterol, procaterol, reproterol, rimiterol, ritodrine, salbutamol, salmeterol, tulobuterol, terbutaline, and xamoterol.

7. The method of any of claim 4, wherein said BAR agonist is selected from Table 1 or 2.

8. The method of any of claim 4, wherein said B-cell proliferative disorder is selected from the group consisting of autoimmune lymphoproliferative disease, B-cell chronic lymphocytic leukemia (CLL), B-cell prolymphocyte leukemia, lymphoplasmacytic lymphoma, mantle cell lymphoma, follicular lymphoma, extranodal marginal zone B-cell lymphoma of mucosa-associated lymphoid tissue (MALT type), nodal marginal zone lymphoma, splenic marginal zone lymphoma, hairy cell leukemia, plasmacytoma, diffuse large B-cell lymphoma, Burkitt lymphoma, multiple myeloma, indolent myeloma, smoldering myeloma, monoclonal gammopathy of unknown significance (MGUS), B-cell non-Hodgkin's lymphoma, small lymphocytic lymphoma, monoclonal immunoglobin deposition diseases, heavy chain diseases, mediastinal (thymic) large B-cell lymphoma, intravascular large B-cell lymphoma, primary effusion lymphoma, lymphomatoid granulomatosis, precursor B-lymphoblastic leukemia/lymphoma, Hodgkin's lymphoma (e.g., nodular lymphocyte predominant Hodgkin's lymphoma, classical Hodgkin's lymphoma, nodular sclerosis Hodgkin's lymphoma, mixed cellularity Hodgkin's lymphoma, lymphocyte-rich classical Hodgkin's lymphoma, and lymphocyte depleted Hodgkin's lymphoma), post-transplant lymphoproliferative disorder, and Waldenstrom's macroglobulinemia.

9. The method of claim 4, wherein said B-cell proliferative disorder is multiple myeloma.

10. The method of claim 4, wherein said second compound is said IL-6 agonist selected from the group consisting of IL-6, cytokines, soluble IL-6 receptor, platelet-derived growth factor, prostaglandin E1, forskolin, cholera toxin, dibutyryl cAMP, and IL-6 receptor agonists.

11. The method of claim 4, wherein, when said B-cell proliferative disorder is mantle cell lymphoma, said BAR agonist is not salmeterol administered with CHOP or bortezomib; when said B-cell proliferative disorder is multiple myeloma, said BAR agonist is not salbutamol administered with VAD; when said B-cell proliferative disorder is multiple myeloma, said BAR agonist is not salmeterol administered with prednisone and melphalan; when said B-cell proliferative disorder is multiple myeloma, said BAR agonist is not salbutamol administered with clodronate; or when said B-cell proliferative disorder is multiple myeloma, said BAR agonist is not salbutamol administered with melphalan, prednisone, and pamidronate for multiple myeloma.

12. The method of claim 4, wherein said patient is not suffering from asthma, bronchiolitis obliterans, COPD, or shortness of breath.

13. The method of claim 4, wherein said patient is not suffering from an immunoinflammatory disorder of the lungs.

14. The method of claim 4, wherein said patient is not suffering from an immunoinflammatory disorder.

15. The method of claim 4, wherein said patient is not preparing to undergo, undergoing, or recovering from allogenic or autologous stem cell replacement.

16. The method of claim 4, wherein said patient is not concomitantly treated with a stem cell mobilizer.

17. The method of claim 4, wherein said patient is not concomitantly treated with an mTOR inhibitor and capecitabine.

18. The method of claim 4, wherein said BAR agonist is not isoproterenol.

19. The method of claim 4, wherein said BAR agonist is formulated for oral or intravenous administration.

20. The method of claim 4, wherein said BAR agonist and said A2A agonist, PDE inhibitor, antiproliferative compound, or IL-6 agonist are administered simultaneously.

21. The method of claim 4, wherein said BAR agonist and said A2A agonist, PDE inhibitor, antiproliferative compound, or IL-6 agonist are administered within 14 days of one another.

22. The method of claim 4, wherein said second compound is said A2A agonist selected from Table 3 or 4, or said second compound is said PDE inhibitor selected from Table 5 or 6.

23. The method of claim 4, wherein said second compound is said antiproliferative compound selected from the group consisting of alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin A receptor antagonist, retinoic acid receptor agonists, immuno-modulators, hormonal and antihormonal agents, photodynamic agents, tyrosine kinase inhibitors, antisense compounds, corticosteroids, HSP90 inhibitors, proteosome inhibitors, CD40 inhibitors, anti-CSI antibodies, FGFR3 inhibitors, VEGF inhibitors, MEK inhibitors, cyclin D inhibitors, NF-kB inhibitors and pathway modulators, anthracyclines, histone deacetylase inhibitors, kinesin inhibitors, phosphatase inhibitors, COX2 inhibitors, mTOR inhibitors, AKT inhibitors, PI3K inhibitors, TRAF inhibitors, statins, mitotic kinase inhibitors, KSP inhibitors, cyclin dependent kinase inhibitors, inhibitors of anti-apoptotic proteins, immune therapies, calcineurin antagonists, and IMiDs.

24. The method of claim 4, wherein said second compound is said antiproliferative compound selected from Table 7 or 8.

25. The method of claim 4, wherein said second compound is said antiproliferative compound and the combination of BAR agonist and antiproliferative compound is selected from Table 9.

26. The method of claim 4, wherein said BAR agonist is a beta 2 agonist.

27. (canceled)

28. (canceled)

29. (canceled)

30. (canceled)

31. (canceled)

32. A pharmaceutical composition comprising a BAR agonist and a second compound selected from the group consisting of an A2A agonist, a PDE inhibitor, an antiproliferative compound, and an IL-6 agonist, in an amount effective to treat a B-cell proliferative disorder.

33. The composition of claim 32, wherein said second compound is said A2A agonist selected from Table 3 or 4, or said second compound is said PDE inhibitor selected from Table 5 or 6.

34. The composition of claim 32, wherein said second compound is said antiproliferative compound selected from the group consisting of alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin A receptor antagonist, retinoic acid receptor agonists, immuno-modulators, hormonal and antihormonal agents, photodynamic agents, tyrosine kinase inhibitors, antisense compounds, corticosteroids, HSP90 inhibitors, proteosome inhibitors, CD40 inhibitors, anti-CSI antibodies, FGFR3 inhibitors, VEGF inhibitors, MEK inhibitors, cyclin D inhibitors, NF-kB inhibitors and pathway modulators, anthracyclines, histone deacetylase inhibitors, kinesin inhibitors, phosphatase inhibitors, COX2 inhibitors, mTOR inhibitors, AKT inhibitors, PI3K inhibitors, TRAF inhibitors, statins, mitotic kinase inhibitors, KSP inhibitors, cyclin dependent kinase inhibitors, inhibitors of anti-apoptotic proteins, immune therapies, calcineurin antagonists, and IMiDs.

35. The composition of claim 32, wherein said second compound is said antiproliferative compound selected from Table 7 or 8.

36. The composition of claim 32, wherein said second compound is said antiproliferative compound and the combination of BAR agonist and antiproliferative compound is selected from Table 9.

37-48. (canceled)

49. The composition of claim 32, wherein said BAR agonist is arbutaline, arfomoterol, bambuterol, bitolterol, broxaterol, clenbuterol, fenoterol, formoterol, hexoprenaline, indacaterol, isoetharine, isoproterenol, levalbuterol, meluadrine, metaproterenol, nylidrin, picumeterol, pirbuterol, procaterol, reproterol, rimiterol, ritodrine, salbutamol, salmeterol, tulobuterol, terbutaline, and xamoterol.

50. The composition of claim 32, wherein said BAR agonist is selected from Table 1 or 2.

51. The composition of claim 32, wherein said second compound is said IL-6 agonist selected from the group consisting of IL-6, cytokines, soluble IL-6 receptor, platelet-derived growth factor, prostaglandin E1, forskolin, cholera toxin, dibutyryl cAMP, and IL-6 receptor agonists.
